(19) United States
(12) Patent Application Publication
(10) Pub. No.: US 2022/0142919 A1
(43) Pub. Date: May 12, 2022

(54) DYNAMIC COVALENTLY LINKED HYDROGELS AS STABILIZATION NETWORK PLATFORMS

(71) Applicant: NANOLY BIOSCIENCE, INC., Denver, CO (US)

(72) Inventors: Bruno Marco-Dufort, Zurich (CH); Mark W. Tibbitt, Zurich (CH); Balaji V. Sridhar, Denver, CO (US); John R. Janczy, Denver, CO (US); David Busha, Boulder, CO (US); Margaret Best, Boulder, CO (US)

(73) Assignee: Nanoly Bioscience, Inc., Denver, CO (US)

(21) Appl. No.: 17/433,736

(22) PCT Filed: Mar. 5, 2020
(86) PCT No.: PCT/US2020/021234

§ 371 Date: Aug. 25, 2021

Related U.S. Application Data
(60) Provisional application No. 62/814,547, filed on Mar. 6, 2019.

(51) Int. Cl. A61K 9/06; A61K 47/34; A61K 35/54; A61K 38/46; A61K 38/20; A61K 38/19; A61K 35/761; A61K 39/395; A61K 38/52; A61K 38/47

(52) U.S. Cl. CPC A61K 9/06; A61K 47/34; A61K 35/54; A61K 38/465; A61K 38/47; A61K 38/191; A61K 35/761; A61K 39/3955; A61K 38/52; A61K 38/208

(57) ABSTRACT

The present invention provides dynamically covalent polymeric hydrogel systems for encapsulating and stabilizing bioactive therapeutic agents (e.g., proteins, cells, viruses, and vaccines) from environmental stressors, obviating standard refrigeration requirements, and decreasing transportation and storage costs of temperature-sensitive biomolecules. Described herein are dynamic polymeric hydrogel compositions comprising a therapeutic agent and a combination of phenylboronic acid- and 1,2-diol-modified multi-arm polyethylene glycol (PEG) polymer backbones. Methods of encapsulating and stabilizing bioactive therapeutic agents within the dynamic polymeric hydrogel compositions are also provided. Also described are methods for releasing stabilized therapeutic agents from hydrogel encapsulation. The covalently adaptable hydrogel release systems allow for discretionary administration of temperature-sensitive therapeutic agents, as well as the parenteral administration of highly concentrated amounts of therapeutic agents.

### CROSS-REFERENCE TO RELATED APPLICATIONS

The present application claims the benefit of priority to U.S. Provisional Application No. 62/814,547, filed Mar. 6, 2019, the full disclosure of which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant No. 1R43GM128466-01 awarded by the National Institutes of Health, The National Institute of General Medical Sciences. The Government has certain rights in the invention.

### BACKGROUND OF THE INVENTION

Stabilization and long-term storage of therapeutic agents is a critical feature of many applications, because the therapeutic agents are usually labile and sensitive to changes in surrounding conditions, e.g., temperature, humidity, and/or light. In particular, vaccine stabilization has been a long-lasting challenge and large amounts of vaccines have been wasted due to improper storage. Vaccines are temperature-sensitive biological substances that may lose their effectiveness quickly if they become too hot or too cold, especially during transport and storage, and need to be stabilized from environmental stressors. Inadvertent freezing, heating above 8° C. or other breaks in the cold chain may result in either failure of efficacy or vaccine wastage. According to the WHO, between 2006-2015, the U.S. alone will have contributed $35 billion for global vaccination programs. About one third will be spent on vaccines and remainder will be spent on vaccine delivery systems. It is clear that even 1% vaccine wastage because of cold chain failure is a considerable sum. Indeed, for five U.S. states, the average wastage of 1% to 5% cost approximately $6-$31 million. In other parts of the world, vaccine wastage can reach 10%. The two most common forms of wastage relate to heat stability and shelf life.

Thus, there is a growing need for storage-stable therapeutic agents with longer shelf life that can maintain efficacy under various robust environmental conditions. Refrigeration (i.e., cold chain compliance) and lyophilization are too cost prohibitve and have limited access to many people worldwide for life-saving medicines. Utilizing a synthetic material to circumvent the need for refrigeration would greatly reduce costs and increase access of therapeutics to millions worldwide.

One type of synthetic material that has received considerable attention for medical applications is the polymeric hydrogel. Hydrogels are three-dimensional polymer networks composed of homopolymers or copolymers that are capable of absorbing large amounts of water. Thus, a characteristic of hydrogels is that they swell in water or aqueous fluids without dissolving. High water content and soft consistency make hydrogels similar to natural living tissue more than any other class of synthetic biomaterials. Accordingly, many hydrogels are compatible with living systems and hydrogels have found numerous applications in medical and pharmaceutical industries. For example, hydrogels have been investigated widely as drug carriers due to their adjustable swelling capacities, which permit flexible control of drug release rates.

However, a disadvantage of some polymeric hydrogel compositions is that the hydrogels must be formed before they can be used. This is applicable to polymeric compositions that require cross-linking, which is the formation of a linkage (e.g., covalent, non-covalent, or combinations thereof) between polymer chains or between portions of the same polymer chain. Cross-linking is frequently accomplished through the introduction of a cross-linker that has functionality capable of reacting chemically with functionality on one or more polymer chains. For hydrogels, the polymer network is created by forming cross-links between polymeric chains. For many polymeric compositions, extreme conditions and reactive cross-linkers are required for crosslinking. Such conditions are not generally compatible with certain environments (e.g., uses in biological systems or preserving sensitive therapeutic agents). For example, the preparation of some polymer hydrogels can require high temperature, exotic reagents, initiators, and/or solvents, and expensive and/or toxic catalysts, which, instead of forming the desired polymer network, can react with cells, tissues, biomolecules, and other species present in a given application.

It can be desirable in certain applications to have cross-linking that is reversible, e.g., one or more cross-links can be formed, broken, and reformed in the same or different location in the polymer network. Gels that dynamically restructure are commonly observed in nature, including synovial fluid (Balazs and Gibbs, Chem Mol Biol Intercell Matrix, Advan Study Inst 3:1241-53, 1970; Gibbs et al., Biopolymers 6:777-91, 1968) and mucins (Pearson et al., Methods in Molecular Biology, 125:99-109, 2000). Such materials are the subject of intense investigation for fundamental material science and advanced biomaterial applications, such as artificial biofluids and biosolids, cell encapsulation, tissue engineering and injectable drug delivery.

A critical aspect of designing a biomaterial to thermally stabilize relevant therapeutics is tuning and controlling the degradation behavior of materials. Conventional degradation technology uses hydrolysis and/or enzymatic degradation, which are sustained processes that offer minimal spatial or temporal control. Most synthetic biomaterials degrade via hydrolysis, which can occur throughout the bulk or only at the surface of a biomaterial and leads to a sustained and non-instantaneous mass loss, which may be undesirable. Current photopolymerization and photodegradation techniques require the use of a photosensitizer, and often have no spatial control. There is a need for an improved formation and degradation process that allows for increased user control and the ability to thermally stabilize therapeutics. The present invention satisfies these and other needs.

### BRIEF SUMMARY OF THE INVENTION

In one aspect, the present invention provides a method for stabilizing a therapeutic agent comprising encapsulating the therapeutic agent in a dynamic polymeric hydrogel composition. The dynamic polymeric hydrogel composition comprises a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II):

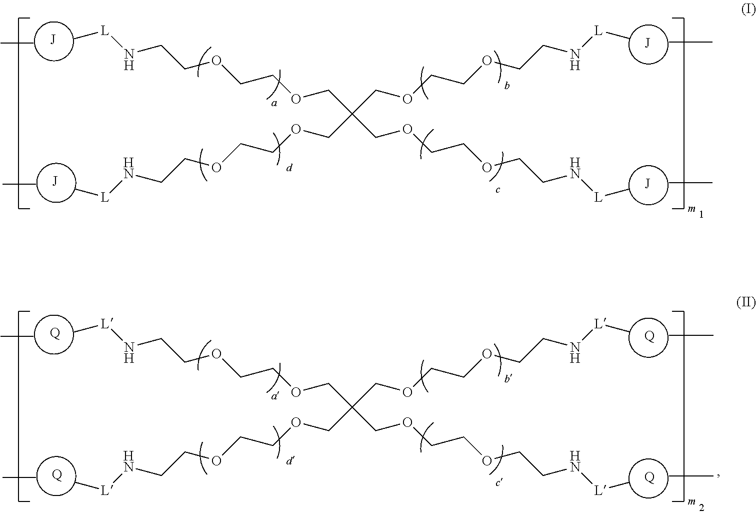

wherein subscripts a, a′, b, b′, c, c′, d, and d′ are each independently an integer selected from 1 to 500; subscripts m₁ and m₂ are each independently an integer selected from 10 to 20,000; linkers L and L′ are each independently selected from a bond, —C(O)—, substituted or unsubstituted alkylene, substituted or unsubstituted —C(O)-alkylene, and substituted or unsubstituted heteroalkylene; each J is a phenylboronic acid derivative; and each Q is a 1,2-diol moiety; and wherein the PBA modified multi-arm PEG polymer backbone and the 1,2-diol modified multi-arm PEG polymer backbone are reversibly covalently cross-linked through the phenylboronic acid derivatives and the 1,2-diol groups.

In some embodiments, the dynamic polymeric hydrogel composition used in the methods for stabilizing a therapeutic agent comprises a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II) wherein subscripts a, a′, b, b′, c, c′, d, and d′ are each independently an integer selected from 10 to 250; subscripts m₁ and m₂ are each independently an integer selected from 25 to 10,000; each linker L is selected from a bond, —C(O)—, substituted or unsubstituted C₁₋₆ alkylene, and unsubstituted —C(O)—C₁₋₆ alkylene, wherein substituted C₁₋₆ alkylene is substituted with at least one substituent selected from —OH, —NH₂, —SH, —CN, —CF₃, —COOH, —C(O)NH₂, halogen, unsubstituted C₁₋₃ alkyl, and substituted C₁₋₃ alkyl; and each linker L′ is selected from a bond, —C(O)—, unsubstituted C₁₋₆ alkylene, and unsubstituted —C(O)—C₁₋₆ alkylene.

In some embodiments, the phenylboronic acid derivative of the PBA modified multi-arm PEG polymer backbone used in the methods for stabilizing a therapeutic agent comprises a phenylboronic acid group of formula (III):

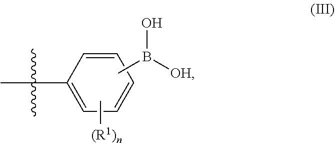

wherein each R¹ is each independently selected from the group consisting of substituted or unsubstituted alkyl, substituted or unsubstituted heteroalkyl, halogen, —CN, —OH, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; R^{a} and R^{b} are each independently selected from the group consisting of hydrogen and C₁₋₆ alkyl; and subscript n is an integer from 0-4; wherein the pKa of the phenylboronic acid group is less than 7.8.

In some embodiments, the phenylboronic acid derivative of the PBA modified multi-arm PEG polymer backbone used in the methods for stabilizing a therapeutic agent comprises the phenylboronic acid group of formula (III) wherein each R¹ is each independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b}; R^{a} and R^{b} are each independently selected from the group consisting of hydrogen and C₁₋₆ alkyl; and subscript n is an integer from 0-3; wherein the pKa of the phenylboronic acid group is between about 3.5 and about 7.4.

In some embodiments, the phenylboronic acid derivative of the PBA modified multi-arm PEG polymer backbone used in the methods for stabilizing a therapeutic agent comprises a phenylboronic acid group of formula (IIIA) or formula (IIIB):

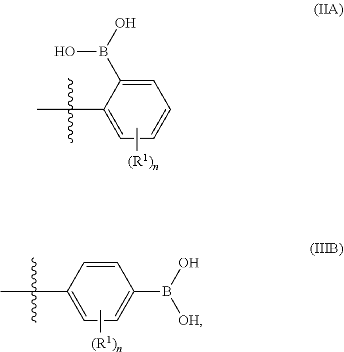

wherein each R¹ is each independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NR^{a}R^{b}, —CH₂NR^{a}R^{b}, —C(O)R^{a}, and —C(O)OR^{a}; R^{a} and R^{b} are each independently selected from the group consisting of hydrogen and C₁₋₃ alkyl; and subscript n is an integer from 0-2; wherein the pKa of the phenylboronic acid group is between about 4.0 and about 7.2.

In some embodiments, the 1,2-diol moiety of the 1,2-diol modified multi-arm PEG polymer backbone used in the methods for stabilizing a therapeutic agent comprises a 1,2-cis-diol group of formula (IV):

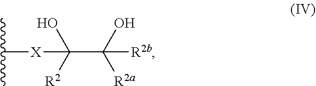

wherein R², R^{2a}, and R^{2b} are each independently selected from the group consisting of hydrogen, substituted or unsubstituted alkyl, substituted or unsubstituted heteroalkyl, halogen, —CN, —OH, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; wherein, optionally, R² and one of R^{2a} or R^{2b} are combined together to form a substituted or unsubstituted cycloalkyl, substituted or unsubstituted heterocycloalkyl, substituted or unsubstituted aryl, or substituted or unsubstituted heteroaryl; R^{a} and R^{b} are each independently selected from the group consisting of hydrogen and C₁₋₆ alkyl; and X is selected from the group consisting of a bond, substituted or unsubstituted alkylene, substituted or unsubstituted heteroalkylene, and —C(O)—.

In some embodiments, the 1,2-diol moiety of the 1,2-diol modified multi-arm PEG polymer backbone used in the methods for stabilizing a therapeutic agent comprises the 1,2-cis-diol group of formula (IV) wherein R², R²a, and R^{2b} are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, —NR^{a}R^{b}, —CH₂NR^{a}R^{b}, —C(O)R^{a}, —C(O)OR^{a}, and —C(O)NR^{a}R^{b}; wherein, optionally, R² and one of R^{2a} or R^{2b} are combined together to form a substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₆ alkyl; and X is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—.

In some embodiments, the 1,2-diol moiety of the 1,2-diol modified multi-arm PEG polymer backbone used in the methods for stabilizing a therapeutic agent comprises a 1,2-cis-diol group of formula (IVA):

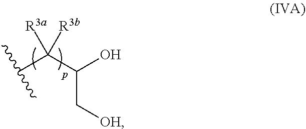

wherein R^{3a} and R^{3b} are each independently selected from the group consisting of hydrogen, substituted or unsubstituted C₁₋₃ alkyl, methoxy, ethoxy, fluoro, chloro, bromo, iodo, —OH, —NR^{a}R^{b}, —CH₂NR^{a}R^{b}, —C(O)R^{a}, and —C(O)OR^{a}; wherein, optionally, one or more of R³a and R^{3b} are combined together to form ═O, substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₃ alkyl; and subscript p is an integer from 1-6.

The methods for stabilizing a therapeutic agent involves encapsulating the therapeutic agent in a dynamic polymeric hydrogel composition, wherein encapsulating the therapeutic agent comprises (a) admixing the therapeutic agent with a solution of the 1,2-diol modified multi-arm PEG polymer backbone of formula (II) to form a therapeutic agent diol-PEG admixture; and (b) adding a solution of the PBA modified multi-arm PEG polymer backbone of formula (I) to the therapeutic agent diol-PEG admixture to form the dynamic polymeric hydrogel composition with the therapeutic agent encapsulated therein, thereby stabilizing the encapsulated therapeutic agent. In some embodiments, the stoichiometric ratio of the PBA modified multi-arm PEG polymer backbone of formula (I) to the 1,2-diol modified multi-arm PEG polymer backbone of formula (II) ranges from 1:5 to 5:1.

In some embodiments, the therapeutic agent used in the stabilization and encapsulation methods described herein is present in an amount of from about 0.10 mg/mL to about 100 mg/mL. In some embodiments, the therapeutic agent is selected from the group consisting of an enzyme, cell therapy, antibiotic, anesthetic, antibody, growth factor, human embryonic cells, protein, hormone, anti-inflammatory agent, analgesic, cardiac agent, vaccine, and psychotropic agent. In some embodiments, the therapeutic agent is selected from the group consisting of β-galactosidase, a vaccine, Topoisomerase I-IV, HEK293 cells, DNA gyrase, adalimumab, anti-CD3 monoclonal antibody, ustekinumab, TNFα, interleukin 12 (Il-12), influenza vaccine, eukaryotic or prokaryotic cells, adenovirus, and adeno-associated virus.

In a related aspect, the invention provides a method for releasing a stabilized therapeutic agent encapsulated in the dynamic polymeric hydrogel composition comprising (i) adding a sugar solution to the dynamic polymeric hydrogel composition with the encapsulated therapeutic agent; or (ii) lowering the pH of the dynamic polymeric hydrogel composition with the encapsulated therapeutic agent, thereby releasing the stabilized therapeutic agent from the dynamic polymeric hydrogel composition. In some embodiments, the stabilized therapeutic agent is administered to a patient in need thereof after releasing the stabilized therapeutic agent from the dynamic polymeric hydrogel composition.

In another aspect, the invention provides a dynamic polymeric hydrogel composition comprising (a) a therapeutic agent selected from the group comprising 3-galactosidase, a vaccine, Topoisomerase I-IV, HEK293 cells, DNA gyrase, adalimumab, anti-CD3 monoclonal antibody, ustekinumab, TNFα, Il-12, influenza vaccine, eukaryotic or prokaryotic cells, adenovirus, and adeno-associated virus; wherein the therapeutic agent is present in an amount of from about 0.10 mg/mL to about 100 mg/mL; and (b) a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II):

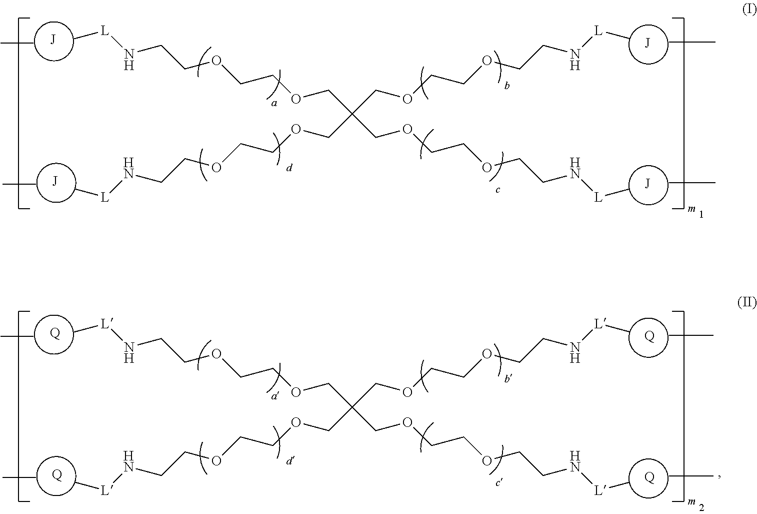

wherein subscripts a, a′, b, b′, c, c′, d, and d′ are each independently an integer selected from 1 to 500; subscripts m₁ and m₂ are each independently an integer selected from 10 to 20,000; linkers L and L′ are each independently selected from a bond, —C(O)—, substituted or unsubstituted alkylene, substituted or unsubstituted —C(O)-alkylene, and substituted or unsubstituted heteroalkylene; each J is a phenylboronic acid derivative having a pKa of less than 7.8; and each Q is a 1,2-diol moiety; and wherein the PBA modified multi-arm PEG polymer backbone and the 1,2-diol modified multi-arm PEG polymer backbone are reversibly covalently cross-linked through the phenylboronic acid derivatives and the 1,2-diol groups.

In some embodiments, the dynamic polymeric hydrogel composition comprises a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II) wherein subscripts a, a′, b, b′, c, c′, d, and d′ are each independently an integer selected from 10 to 250; subscripts m₁ and m₂ are each independently an integer selected from 25 to 10,000; each linker L is selected from a bond, —C(O)—, substituted or unsubstituted C₁₋₆ alkylene, and unsubstituted —C(O)—C₁₋₆ alkylene, wherein substituted C₁₋₆ alkylene is substituted with at least one substituent selected from —OH, —NH₂, —SH, —CN, —CF₃, —COOH, —C(O)NH₂, halogen, unsubstituted C₁₋₃ alkyl, and substituted C₁₋₃ alkyl; and each linker L′ is selected from a bond, —C(O)—, unsubstituted C₁₋₆ alkylene, and unsubstituted —C(O)—C₁₋₆ alkylene.

In some embodiments, the phenylboronic acid derivative of the PBA modified multi-arm PEG polymer backbone of the dynamic polymeric hydrogel composition comprises a phenylboronic acid group of formula (III):

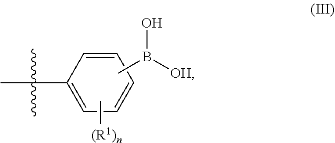

wherein each R¹ is each independently selected from the group consisting of substituted or unsubstituted alkyl, substituted or unsubstituted heteroalkyl, halogen, —CN, —OH, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; R^{a} and R^{b} are each independently selected from the group consisting of hydrogen and C₁₋₆ alkyl; and subscript n is an integer from 0-4; wherein the pKa of the phenylboronic acid group is less than 7.8.

In some embodiments, the phenylboronic acid derivative of the PBA modified multi-arm PEG polymer backbone of the dynamic polymeric hydrogel composition comprises a phenylboronic acid group of formula (III) wherein each R¹ is each independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b}; R^{a} and R^{b} are each independently selected from the group consisting of hydrogen and C₁₋₆ alkyl; and subscript n is an integer from 0-3; wherein the pKa of the phenylboronic acid group is between about 3.5 and about 7.4.

In some embodiments, the phenylboronic acid derivative of the PBA modified multi-arm PEG polymer backbone of the dynamic polymeric hydrogel composition comprises a phenylboronic acid group of formula (IIIA) or formula (IIIB):

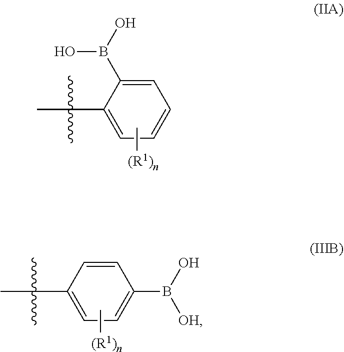

wherein each R¹ is each independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NR^{a}R^{b}, —CH₂NR^{a}R^{b}, —C(O)R^{a}, and —C(O)OR^{a}; R^{a} and R^{b} are each independently selected from the group consisting of hydrogen and C₁₋₃ alkyl; and subscript n is an integer from 0-2; wherein the pKa of the phenylboronic acid group is between about 4.0 and about 7.2.

In some embodiments, the 1,2-diol moiety of the 1,2-diol modified multi-arm PEG polymer backbone of the dynamic polymeric hydrogel composition comprises a 1,2-cis-diol group of formula (IV):

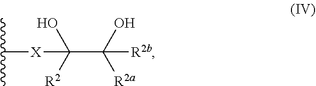

wherein R², R²a, and R^{2b} are each independently selected from the group consisting of hydrogen, substituted or unsubstituted alkyl, substituted or unsubstituted heteroalkyl, halogen, —CN, —OH, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; wherein, optionally, R² and one of R²a or R^{2b} are combined together to form a substituted or unsubstituted cycloalkyl, substituted or unsubstituted heterocycloalkyl, substituted or unsubstituted aryl, or substituted or unsubstituted heteroaryl; R^{a} and R^{b} are each independently selected from the group consisting of hydrogen and C₁₋₆ alkyl; and X is selected from the group consisting of a bond, substituted or unsubstituted alkylene, substituted or unsubstituted heteroalkylene, and —C(O)—.

In some embodiments, the 1,2-diol moiety of the 1,2-diol modified multi-arm PEG polymer backbone of the dynamic polymeric hydrogel composition comprises the 1,2-cis-diol group of formula (IV) wherein R², R²a, and R^{2b} are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, —NR^{a}R^{b}, —CH₂NR^{a}R^{b}, —C(O)R^{a}, —C(O)OR^{a}, and —C(O)NR^{a}R^{b}; wherein, optionally, R² and one of R²a or R^{2b} are combined together to form a substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₆ alkyl; and X is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—.

In some embodiments, the 1,2-diol moiety of the 1,2-diol modified multi-arm PEG polymer backbone of the dynamic polymeric hydrogel composition comprises a 1,2-cis-diol group of formula (IVA):

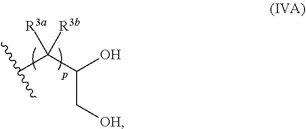

wherein R^{3a} and R^{3b} are each independently selected from the group consisting of hydrogen, substituted or unsubstituted C₁₋₃ alkyl, methoxy, ethoxy, fluoro, chloro, bromo, iodo, —OH, —NR^{a}R^{b}, —CH₂NR^{a}R^{b}, —C(O)R^{a}, and —C(O)OR^{a}; wherein, optionally, one or more of R³a and R^{3b} are combined together to form ═O, substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₃ alkyl; and subscript p is an integer from 1-6.

### BRIEF DESCRIPTION OF THE DRAWINGS

FIG. 1 shows time-dependent shear-thinning and self-healing properties of APBA-1,2-diol cross-linked PEG polymer hydrogels (10 w/v % in PBS, pH=7.4) under low shear strain (ω=10 rad/s, γA=0.1%, 100 s) and high shear strain (ω=10 rad/s, γA=500%, 10 s).

FIG. 2 shows the normalized release of β-galactosidase from within the network of APBA-1,2-diol cross-linked PEG polymer backbones of the dynamic polymeric hydrogel composition over time in 200 mg/mL dextrose solution.

FIG. 3 shows stability of β-galactosidase samples stored at 50° C. for 3 days with and without hydrogel encapsulation. Sample 1: β-galactosidase encapsulated within the APBA-1,2-diol cross-linked PEG hydrogel composition; Sample 2: β-galactosidase in buffer solution (non-encapsulated); Sample 3: β-galactosidase in APBA-PEG solution (non-encapsulated); and Sample 4: β-galactosidase in 1,2-diol-PEG solution (non-encapsulated).

FIG. 4 shows stability of β-galactosidase samples stored at 50° C. after 1 day, 3 days, 6 days, and 14 days with and without hydrogel encapsulation. Sample 1A: 3-galactosidase encapsulated within the APBA-1,2-diol cross-linked PEG hydrogel composition; Sample 2A: β-galactosidase in buffer solution (non-encapsulated); and Sample 3A: Lyophilized β-galactosidase powder containing 70-90% excipients, as purchased from Sigma-Aldrich.

FIG. 5 shows stability of DNA gyrase samples stored at 50° C. and −80° C. for 1 hour with and without hydrogel encapsulation. Hydrogel (−): DNA gyrase in buffer solution (non-encapsulated); Hydrogel (+): DNA gyrase encapsulated within the APBA-1,2-diol cross-linked PEG hydrogel composition (encapsulated).

FIG. 6 shows stability of TopIV samples stored at 50° C. and −80° C. for 1 hour with and without hydrogel encapsulation. Hydrogel (−): TopIV in buffer solution (non-encapsulated); Hydrogel (+): TopIV encapsulated within the APBA-1,2-diol cross-linked PEG hydrogel composition (encapsulated).

FIG. 7 shows stability of DNA gyrase samples stored at 27° C. for 4, 6, and 8 weeks with and without hydrogel encapsulation.

FIG. 8 shows stability of vacuum dried HUMIRA® samples incubated at 65° C. for 24 hours with and without hydrogel encapsulation based on TNFα inhibition assays.

FIG. 9 shows stability of not dried HUMIRA® samples incubated at 65° C. for 24 hours with and without hydrogel encapsulation based on TNFα inhibition assays.

FIG. 10 shows stability of not dried adenovirus (Ad5-GFP) samples stored for 4 hours at room temperature with and without hydrogel encapsulation; stability of dried Ad5-GFP samples stored for 4 hours at room temperature with and without hydrogel encapsulation; and stability of not dried Ad5-GFP samples subjected to 5 freeze/thaw cycles with and without hydrogel encapsulation.

FIG. 11 shows stability of recombinant human TNFα samples after storage for three days at 4° C., room temperature, 37° C. with and without hydrogel encapsulation; and stability of TNFα samples after 5 freeze/thaw cycles with and without hydrogel encapsulation.

FIG. 12 shows stability of recombinant human IL-12 samples after storage for three days at 4° C., room temperature, 37° C. with and without hydrogel encapsulation; and stability of IL-12 samples after 5 freeze/thaw cycles with and without hydrogel encapsulation.

### DETAILED DESCRIPTION OF THE INVENTION

### I. Definitions

As used herein, the terms “stabilizing,” “stabilize,” or “stability” refer to the retention or maintenance of a therapeutic agent's original bioactivity or potency after storage over a defined or indefinite period of time. In the context of the present invention, a therapeutic agent encapsulated within a dynamic polymeric hydrogel is resistant to environmental stressors during storage. The “stable” (i.e., encapsulated) therapeutic agents of the invention retain at least 50% of the therapeutic agents' original bioactivity as compared to “unstable” (i.e., non-encapsulated) therapeutic agents, which have been stored under identical environmental stressors. For example, when a therapeutic agent is encapsulated within the dynamic polymeric hydrogel as described here, the therapeutic agent retains at least 50% or greater percentage of its original bioactivity compared to the un-encapsulated therapeutic agent, which may retain only 20% of its original bioactivity at best. Those skilled in the art appreciate that the percent of bioactivity that is retained is therapeutic agent and stress dependent. Furthermore, the length of time that an encapsulated therapeutic agent is able to maintain its bioactivity or function compared to a naked/un-encapsulated therapeutic agent varies depending on the environmental stressors it is subjected to. The stability of therapeutic agents can be assessed by degrees of aggregation, degradation or fragmentation by methods known to those skilled in the art.

As used herein, the terms “original bioactivity” or “original potency,” with respect to a therapeutic agent, refer to the bioactivity or potency of a therapeutic agent as measured immediately before or immediately after the therapeutic agent is encapsulated within the dynamic polymeric hydrogel. The original bioactivity or potency of a therapeutic agent can also refer to the bioactivity or potency of either an encapsulated therapeutic agent or un-encapsulated therapeutic agent as measured immediately before storage and/or being subjected to environmental stressors.

As used herein, the terms “environmental stressor” or “stressful environment” refer to the external conditions which will reduce original bioactivity or potency of a therapeutic agent. A stressful environment may include various manufacture, preparation, transportation and/or storage conditions, such as temperatures which create adverse thermal environments which could be elevated or reduced temperatures, a change in ambient air pressure, light condition, humidity, solvents such as an organic solvent, the presence of proteases, pH, and/or lack of buffer.

As used herein, the term “therapeutic agent” refers to a chemical compound or biological molecule that can be used to treat a condition, disease, or disorder or reduce the symptoms of the condition, disease, or disorder. In a more general sense, a therapeutic agent refers to any chemical compound or biological molecule that causes a measurable physiological response in a mammal. The mammal may be human or non-human. Therapeutic agents include, without limitation, enzymes, antibiotics, anesthetics, antibodies, cells, growth factors, nucleic acids, peptides, proteins, hormones, anti-inflammatories, analgesics, cardiac agents, vaccines, and psychotropics.

As used herein, the terms “encapsulating” or “encapsulate” refer to the confinement of a therapeutic agent within a material, in particular, within a dynamic polymeric hydrogel. The term “co-encapsulation” refers to encapsulation of more than one therapeutic agent within the material, e.g., the dynamic polymeric hydrogel.

As used herein, the phrase “dynamic polymeric hydrogel composition” refers to a network of polyethylene glycol (PEG) polymers formed upon the combination of a phenylboronic acid (PBA) modified multi-arm PEG polymer backbone and a 1,2-diol modified multi-arm PEG polymer backbone. The PBA modified PEG polymer and the 1,2-diol modified PEG polymer of the hydrogel network are reversibly cross-linked via covalent interactions between the PBA derivative and the 1,2-diol moiety of the modified PEG polymer backbones. In some instances, the dynamic polymeric hydrogel composition refers to the hydrogel network comprising the combination of PBA modified multi-arm PEG polymer backbone components, 1,2-diol modified multi-arm PEG polymer backbone components, a one or more therapeutic agent, and, optionally, one or more pharmaceutically acceptable excipients.

As used herein, the term “linker” refers to a moiety through which a PBA derivative or a 1,2-diol moiety is covalently attached to the polymer backbone of the dynamic polymeric hydrogel composition described herein. Examples of linkers include, but are not limited to, a bond, —C(O)—, —C(O)O—, —C(O)NH—, substituted or unsubstituted alkylene, substituted or unsubstituted —C(O)-alkylene, substituted or unsubstituted —C(O)O-alkylene, substituted or unsubstituted —C(O)NH-alkylene, and substituted or unsubstituted heteroalkylene.

Where chemical substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups can be substituted or unsubstituted. For example, “substituted alkyl” groups can be substituted with one or more groups selected from halo, hydroxy, amino, alkylamino, amido, acyl, nitro, cyano, and alkoxy.

As used herein, the term “alkoxy,” by itself or as part of another substituent, refers to a group having the formula —OR, wherein R is alkyl.

The term “alkylene,” by itself or as part of another substituent, refers to an alkyl group, as defined above, linking at least two other groups (i.e., a divalent alkyl radical), as exemplified, but not limited, by —CH₂CH₂CH₂CH₂—. The two moieties linked to the alkylene group can be linked to the same carbon atom or different carbon atoms of the alkylene group. An alkylene group can have from 1 to 24 carbon atoms. A “lower alkylene” is a shorter chain alkylene group, generally having eight or fewer carbon atoms. Useful alkylene groups include C₁₋₆ alkylene groups.

As used herein, the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. For example, “substituted cycloalkyl” groups can be substituted with one or more groups selected from halo, hydroxy, amino, alkylamino, amido, acyl, nitro, cyano, and alkoxy.

The term “heteroalkyl,” by itself or as part of another substituent, refers to an alkyl group of any suitable length and having from 1 to 3 heteroatoms such as N, O and S. For example, heteroalkyl can include ethers, thioethers and alkyl-amines. Additional heteroatoms can also be useful, including, but not limited to, B, Al, Si and P. The heteroatoms can be oxidized to form moieties such as, but not limited to, —S(O)— and —S(O)₂—. The heteroatom portion of the heteroalkyl can replace a hydrogen of the alkyl group to form a hydroxy, thio, or amino group. Alternatively, the heteroatom portion can be the connecting atom, or be inserted between two carbon atoms. Useful heteroalkyl groups include 2 to 8 membered heteroalkyl groups.

As used herein, the term “heteroalkylene” refers to a heteroalkyl group, as defined above, linking at least two other groups. The two moieties linked to the heteroalkylene can be linked to the same atom or different atoms of the heteroalkylene. Useful heteroalkylene groups include 2 to 8 membered heteroalkylene groups.

As used herein, the terms “halo” and “halogen,” by themselves or as part of another substituent, refer to a fluorine, chlorine, bromine, or iodine atom.

As used herein, the term “haloalkyl,” by itself or as part of another substituent, refers to an alkyl group where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

As used herein, the term “haloalkoxy,” by itself or as part of another substituent, refers to an alkoxy group where some or all of the hydrogen atoms are replaced with halogen atoms.

As used herein, the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. For example, “substituted aryl” groups can be substituted with one or more groups selected from halo, hydroxy, amino, alkylamino, amido, acyl, nitro, cyano, and alkoxy.

As used herein, the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S.

Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. For example, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

As used herein, the term “heterocycloalkyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. heterocycloalkyl groups can include any number of ring atoms, such as, C₃₋₆, C₄₋₆, C₅₋₆, C₃₋₈, C₄₋₈, C₅₋₈, C₆₋₉, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, “substituted heterocycloalkyl” groups can be substituted with one or more groups selected from halo, hydroxy, amino, oxo, alkylamino, amido, acyl, nitro, cyano, and alkoxy.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents are generally those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In general, “substituted,” as used herein, does not encompass replacement and/or alteration of a key functional group by which a molecule is identified, e.g., such that the “substituted” functional group becomes, through substitution, a different functional group. For example, a “substituted phenyl” group must still comprise the phenyl moiety and cannot be modified by substitution, in this definition, to become, e.g., a cyclohexyl group.

As used herein, the terms “hydrogel” or “hydrogel network” refer to a system of water soluble polymers, water insoluble covalent cross-links, and an aqueous solution that surrounds the polymers. The components of the system can be viewed macroscopically as a unit. The water soluble polymers are cross linked by a chemical bond at the cross linking points so that the water soluble polymers are no longer soluble in the aqueous solution. Even though the cross linked polymers are no longer soluble in the aqueous solution, they are not precipitated from the aqueous solution, which allows the hydrogel to be able to hold a large volume of the aqueous solution while still maintaining its shape.

As used herein, the phrase “modified multi-arm polyethylene glycol (PEG) polymer backbone” refers to a multi-branched polymer comprising 2, 3, 4, 5, 6, 7, 8, 9, or 10 polymeric chains (termed “arms” or “branches”) that radiate out from a central core. Each polymeric chain is comprised of repeating units of poly(ethylene glycol) (PEG). Such multi-arm PEG polymers are commercially available or can be synthesized by methods known in the art. The end of each PEG arm is modified with either a phenylboronic acid derivative or 1,2-diol moiety by methods known in the art.

As used herein, the terms “phenylboronic acid derivative” or “PBA derivative” refer to an arylboronic acid moiety that contains an aryl group, as disclosed herein, substituted with one or more —B(OH)₂ groups. The aryl group of phenylboronic acid derivative can be optionally substituted with up to four substituents in addition to the one or more —B(OH)₂ group(s).

As used herein, the term “1,2-diol moiety” refers to a dihydroxy alcohol chemical entity containing two hydroxyl groups connected to adjacent carbon atoms (i.e., a vicinal diol) of a hydrocarbon group. The 1,2-diol moiety can be a 1,2-cis-diol group, in which both hydroxyl groups are on the same side of the hydrocarbon molecule, or a 1,2-trans-diol group, in which each hydroxyl group is on opposite sides of the hydrocarbon molecule. A hydrocarbon is an art recognized term and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

As used herein, the phrase “reversibly covalently cross-linked,” and formatives thereof, refers to the phenomenon of degradation and reformation of cross-links (i.e., the covalent bond between the PBA derivative and the 1,2-diol moiety, as disclosed herein) in a dynamic polymeric hydrogel composition.

As used herein, the terms “admixing” or “admix” refer to a combination or mixing of a therapeutic agent or more than one therapeutic agent with a component of the pre-gelled dynamic hydrogel composition (i.e., PBA modified PEG polymer backbone or the 1,2-diol modified PEG polymer backbone) in a manner such that components within the population are distributed and evenly dispersed throughout the pre-gelled hydrogel admixture forming either a therapeutic agent diol-PEG admixture or a therapeutic agent PBA-PEG admixture.

As used herein, the term “solution” refers to a mixture in which the components form a single phase in which they are uniformly and stably distributed. The term “suspension” refers to a mixture in which a substance that is insoluble in a liquid, forming a second phase of discrete particles of the substance distributed relatively uniformly, but unstably, within the liquid. By “unstably” it is meant that the phases can separate with time, if left to stand, or under the influence of external forces such as centrifugation, filtration and the like.

As used herein, the term “stoichiometric ratio” when used in the context of dynamic polymeric hydrogel composition refers to the molar ratio of 2 or more components of the dynamic polymeric hydrogel composition, such as, for example, the stoichiometric ratio of the PBA modified multi-arm PEG polymer backbone component and the 1,2-diol modified multi-arm PEG polymer backbone component which form the hydrogel network.

As used herein, the terms “releasing” or “release” refer to disentanglement or “un-encapsulation” of the therapeutic agent(s) from the hydrogel network of the dynamic polymeric hydrogel composition. The therapeutic agent is released from encapsulation from the hydrogel network upon the degradation of cross-links between the PBA derivatives and the 1,2-diol moieties in response to an external stimulus.

As used herein, the terms “external stimulus” or “external stimuli” refer to a change in an environmental characteristic (i.e., physical or chemical change) to which the dynamic polymeric hydrogel composition responds or changes (i.e., degradation or reformation of cross-links). Non-limiting examples of external stimuli include pH change, light, ionic strength change, electric field, magnetic field, hydrolytic activity, enzymatic activity, and solvent or excipient composition change (e.g., exposure to an aqueous sugar solution). The external stimulus is a “trigger” for the dynamic polymeric hydrogel composition in that the external stimulus causes an event that initiates a response or change of the dynamic polymeric hydrogel composition.

As used herein, the terms “administered,” “administering,” or “administration” refer to any form of administration including oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the patient or subject. Suitable routes of administration are well known to the skilled artisan.

As used herein, the phrase “patient in need thereof” refers to a living organism suffering from or prone to a condition that can be treated by administration of a stabilized therapeutic agent, as provided herein. Non-limiting examples include humans, other mammals and other non-mammalian animals.

As used herein, the terms “pharmaceutically acceptable excipient” and “pharmaceutically acceptable additive” refer to a non-toxic material that does not interfere with the effectiveness of the biological activity of the therapeutic agent(s). The pharmaceutically acceptable excipient is an organic or inorganic and natural or synthetic inactive ingredient in a formulation (e.g., dynamic polymeric hydrogel composition), with which one or more active ingredients are combined (e.g., therapeutic agent, PBA modified multi-arm PEG polymer backbone components, and 1,2-diol modified multi-arm PEG polymer backbone components). Such pharmaceutically acceptable excipients can be sterile liquids, such as water, saline, aqueous trehalose, and oils, including those of petroleum, animal, vegetable or synthetic origin, including but not limited to peanut oil, soybean oil, mineral oil, sesame oil and the like. Examples of suitable pharmaceutical excipients are described in “Remington's Pharmaceutical Sciences” by E. W. Martin.

As used herein, the term “therapeutically effective amount” refers to an amount of the therapeutic agent, or multiple therapeutic agents, that is sufficient to treat, alleviate, ameliorate, prevent, relieve, delay onset of, inhibit progression of, reduce severity of, and/or reduce incidence of one or more symptoms or features of the disease, disorder, and/or condition. Those of ordinary skill in the art will appreciate that, in some embodiments, a therapeutically effective amount of a therapeutic agent(s) may be encapsulated and stabilized within the dynamic polymeric hydrogel compositions of the present invention in a single dose (i.e., unit dose), and/or administered in a single dose. In some embodiments, a therapeutic agent(s) may be encapsulated and stabilized and/or administered in a plurality of doses, for example, as part of a dosing regimen.

As used herein, the term “dosing regimen” refers to a set of unit doses (typically more than one) that are administered individually to a subject, typically separated by periods of time, the details of which are described herein.

As used herein, the terms “unit dose” or “dosage unit” or “single dose” refer to a physically discrete unit that contains a predetermined quantity of a therapeutic agent, or multiple therapeutic agents, calculated to achieve an intended effect appropriate for the patient to be treated, the details of which are described herein.

### II. Methods and Compositions

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary.

Thus, if a class of components or moieties A, B, and C are disclosed as well as a class of components or moieties D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

The present invention provides methods and compositions involving dynamic polymeric hydrogels for the stabilization of therapeutic agents. The dynamic polymeric hydrogel compositions comprise a combination of phenylboronic acid (PBA) modified polymer backbones, 1,2-diol modified polymer backbones, and at least one therapeutic agent. The PBA modified polymer backbone and the 1,2-diol modified polymer backbone are reversibly covalently cross-linked through the phenylboronic acid derivatives and the 1,2-diol groups. It is the cross-linking between the PBA and 1,2-diol units that forms the dynamic hydrogel network around the therapeutic agents described herein, encapsulating and stabilizing said therapeutic agents.

### Modified Polymer Backbones

Any suitable polymer backbone structure is useful in the dynamic polymeric hydrogel compositions of the present invention. The polymer backbone structure of the present invention is comprised of one or more repeating units that may be the same or different. In general, polymer backbone structures useful in the dynamic polymeric hydrogel compositions of the present invention are at least partially soluble in aqueous solutions, such as water, buffered salt solutions, or aqueous alcohol solutions. Furthermore, these polymers can be synthetic polymers, natural polymers, or copolymers or blends thereof, and can have charged side groups, or a monovalent ionic salt thereof. Examples of polymers with acidic side groups that can be reacted with cations are poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups are carboxylic acid groups and sulfonic acid groups. Examples of polymers with basic side groups that can be reacted with anions are poly(vinyl amines), poly(vinyl pyridine), poly(vinyl imidazole), and some imino substituted polyphosphazenes. The ammonium or quaternary salt of the polymers can also be formed from the backbone nitrogens or pendant imino groups. Examples of basic side groups are amino and imino groups.

Examples of synthetic polymers include poly(hydroxy acids) such as poly(lactic acid), poly(glycolic acid), and poly(lactic acid-co-glycolic acid); poly(lactide); poly(glycolide); poly(lactide-co-glycolide); polyanhydrides; polyorthoesters; polyamides; polycarbonates; polyalkylenes such as polyethylene and polypropylene; polyalkylene glycols such as poly(ethylene glycol); polyalkylene oxides such as poly(ethylene oxide); polyalkylene terepthalates such as poly(ethylene terephthalate); polyvinyl alcohols; polyvinyl ethers; polyvinyl esters; polyvinyl halides such as poly(vinyl chloride); polyvinylpyrrolidone; polysiloxanes; poly(vinyl alcohols); poly(vinyl acetate); polystyrene; polyurethanes; and copolymers thereof; derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt (jointly referred to herein as “synthetic celluloses”); polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”); poly(butyric acid); poly(valeric acid); and poly(lactide-co-caprolactone); copolymers and blends thereof. As used herein, “derivatives” include polymers having substitutions, additions of chemical groups and other modifications routinely made by those skilled in the art.

Examples of natural polymers include proteins such as albumin, collagen, gelatin and prolamines, for example, zein, and polysaccharides such as alginate, cellulose derivatives and polyhydroxyalkanoates, for example, polyhydroxybutyrate, and copolymers such as poly(lactide-co-glycolide) copolymerized with PEG.

In some embodiments, the polymer backbone structure of the dynamic polymeric hydrogel composition is a polymer selected from alginate, polyphosphazenes, poly(acrylic acids), poly(methacrylic acids), copolymers of acrylic acid and methacrylic acid, polyalkylene glycols, poly(alkylene oxides), poly(vinyl acetate), polyvinylpyrrolidone (PVP), polylactic acid (PLA), polyglycolic acid (PGA), poly(lactic-co-glycolic acid) (PLGA), polyhydroxyalkanoate (PHA), poly(lactic acid)-poly(ethylene oxide) (PLA-PEG), polyanhydrides, poly(ester anhydrides), polymethylmethacrylate (PMMA), poly(2-hydroxyethyl methacrylate) (pHEMA), polycaprolactone (PCL), cellulose acetate, chitosan, and copolymers and blends thereof.

In some embodiments, the polymer backbone structure of the dynamic polymeric hydrogel composition is a polymer comprising repeating units selected from the structures of Table 1 below. In some embodiments, the polymer backbone structure of the dynamic polymeric hydrogel composition is a polymer comprising any combination of repeating structural units, with the understanding that the resulting polymeric backbone structure is at least partially soluble in aqueous solutions.

TABLE 1
Polymeric backbones of the dynamic polymeric hydrogel composition ^{a}

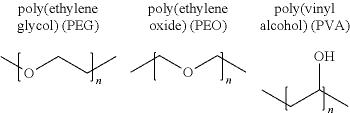

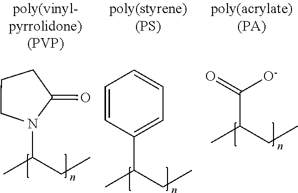

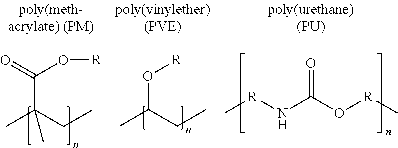

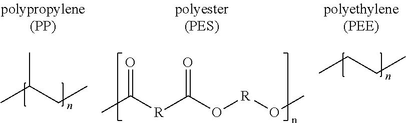

^{a} R is as defined herein; n is 1-200.

In some embodiments, the polymer backbone structure is a polymer comprising repeating units of poly(ethylene glycol) (PEG), poly(ethylene oxide) (PEO), poly(vinyl alcohol) (PVA), poly(vinylpyrrolidone) (PVP), poly(styrene) (PS), poly(acrylate) (PA), poly(methacrylate) (PM), poly(vinylether) (PVE), poly(urethane) (PU), polypropylene (PP), polyester (PES), polyethylene (PEE), polylactic acid (PLA), polyglycolic acid (PGA), poly(lactic-co-glycolic acid) (PLGA), polycaprolactone (PCL), polyhydroxyalkanoate (PHA), poly(lactic acid)-poly(ethylene oxide) (PLA-PEG), polyanhydrides, poly(ester anhydrides), polymethylmethacrylate (PMMA), poly(2-hydroxyethyl methacrylate) (pHEMA), polycaprolactone (PCL), or a mixture thereof. In some embodiments, the polymer backbone structure is a polymer comprising repeating units of PEG, PEO, PVA, PVP, PCL, PS, PEE, PU, PVE, PP, PLA, PMMA, or a mixture thereof. In some embodiments, the polymer backbone structure is a polymer comprising repeating units of PEG, PEO, PVA, PVP, PS, PU, PVE, PP, PLA, PMMA, or a mixture thereof. In some embodiments, the polymer backbone structure is a polymer comprising repeating units of PEG, PEO, PVA, PU, PVE, PLA, or a mixture thereof. In some embodiments, the polymer backbone structure is a polymer comprising repeating units of PEG, PEO, PVA, PLA, or a mixture thereof. In some embodiments, the polymer backbone structure is a polymer comprising repeating units of PEG.

In some embodiments, the polymer backbone structure is a “multi-arm” polymer backbone structure having 2 or more polymeric chains radiating out from a central core. In some embodiments, the multi-arm polymer backbone structure is a 3-arm to 50-arm polymer backbone structure. In some embodiments, the multi-arm polymer backbone structure is a 3-arm to 40-arm polymer backbone structure. In some embodiments, the multi-arm polymer backbone structure is a 3-arm to 25-arm polymer backbone structure. In some embodiments, the multi-arm polymer backbone structure is a 3-arm to 15-arm polymer backbone structure. In some embodiments, the multi-arm polymer backbone structure is a 3-arm to 10-arm polymer backbone structure. In some embodiments, the multi-arm polymer backbone structure is a 3-arm, 4-arm, 5-arm, 6-arm, 7-arm, or 8-arm polymer backbone structure. In some embodiments, the multi-arm polymer backbone structure is a 3-arm, 4-arm, 5-arm, or 6-arm polymer backbone structure. In some embodiments, the multi-arm polymer backbone structure is a 4-arm polymer backbone structure.

The multi-arm polyethylene glycol (PEG) polymer backbone structures of the dynamic polymeric hydrogel compositions of the present invention are modified at the of each PEG arm with either a phenylboronic acid derivative or 1,2-diol moiety by methods known in the art. In some embodiments, the dynamic polymeric hydrogel composition comprises a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II):

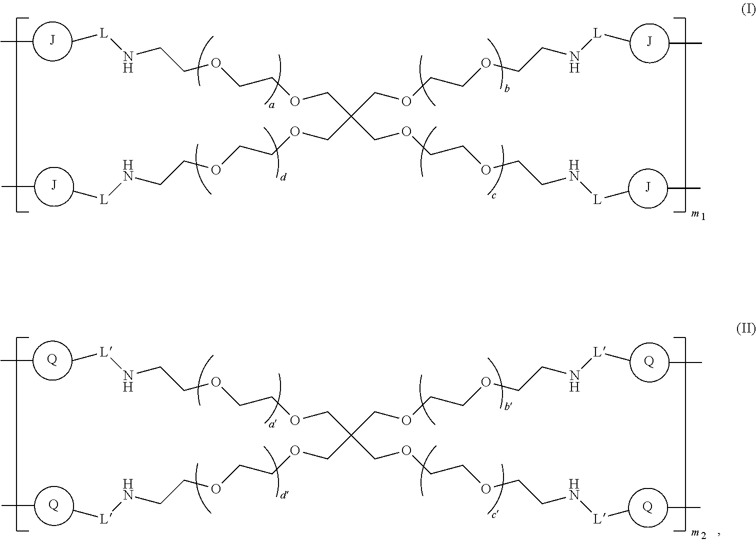

wherein subscripts a, a′, b, b′, c, c′, d, and d′ are each independently an integer selected from 1 to 10,000; subscripts m₁ and m₂ are each independently an integer selected from 1 or greater; linkers L and L′ are each independently selected from a bond, —C(O)—, —C(O)O—, —C(O)NH—, substituted or unsubstituted alkylene, substituted or unsubstituted —C(O)-alkylene, substituted or unsubstituted —C(O)O-alkylene, substituted or unsubstituted —C(O)NH-alkylene, and substituted or unsubstituted heteroalkylene; each J is a phenylboronic acid derivative; and each Q is a 1,2-diol moiety. The PBA modified multi-arm PEG polymer backbone and the 1,2-diol modified multi-arm PEG polymer backbone are reversibly covalently cross-linked through the phenylboronic acid derivatives and the 1,2-diol groups.

In some embodiments, subscripts a, a′, b, b′, c, c′, d, and d′ are each independently an integer selected from 1 to 5,000. In some embodiments, subscripts a, a′, b, b′, c, c′, d, and d′ are each independently an integer selected from 1, 3, 5, 8, 10, 12, 15, 18, 20, 25, 30, 35, 40, 45, 50, 55, 60, 65, 70, 75, 80, 85, 90, 95, 100, 125, 150, 175, 200, 250, 300, 500, 800, 1000, 2500, or 5,000. In some embodiments, subscripts a, a′, b, b′, c, c′, d, and d′ are each independently an integer selected from 3 to 3,000, 5 to 1,500, 8 to 1,000, 10 to 500, 12 to 300, 15 to 250, 18 to 200, 20 to 175, 25 to 150, 30 to 125, 35 to 100, 40 to 95, 45 to 90, 50 to 85, 55 to 80, or 60 to 75. In some embodiments, subscripts a, a′, b, b′, c, c′, d, and d′ are each independently an integer selected from 5 to 1,000, 5 to 500, 10 to 250, 10 to 200, 12 to 180, 12 to 175, 15 to 170, 20 to 150, 25 to 130, 30 to 100, 40 to 85, 45 to 80, or 50 to 65. In some embodiments, subscripts a, a′, b, b′, c, c′, d, and d′ are each independently an integer selected from 10, 12, 20, 25, 50, 55, 100, 150, 170, or 500.

In some embodiments, subscripts m₁ and m₂ are each independently an integer selected from 1 to 1,000,000. In some embodiments, subscripts m₁ and m₂ are each independently an integer selected from 2, 3, 5, 10, 12, 15, 20, 25, 30, 40, 50, 60, 70, 80, 90, 100, 125, 500, 750, 1,000, 2,500, 5,000, 7,500, 10,000, 20,000, 25,000, 30,000, 50,000, 60,000, 75,000, 80,000, 100,000, 250,000, 500,000 or 750,000. In some embodiments, subscripts m₁ and m₂ are each independently an integer selected from 2 to 750,000, 5 to 500,000, 8 to 125,000, 10 to 100,000, 12 to 75,000, 15 to 50,000, 18 to 25,000, 20 to 20,000, 25 to 15,000, 30 to 12,500, 35 to 10,000, 40 to 7,000, 45 to 5,000, 50 to 1,000, 55 to 750, or 60 to 500. In some embodiments, subscripts m₁ and m₂ are each independently an integer selected from 5 to 75,000, 5 to 55,000, 8 to 50,000, 8 to 40,000, 10 to 35,000, 10 to 25,000, 12 to 20,000, 12 to 18,000, 15 to 17,500, 15 to 17,000, 20 to 15,500, 20 to 15,000, 25 to 13,000, 30 to 12,000, 35 to 10,000, 40 to 8,000, 45 to 5,000, or 50 to 1,000.

In some embodiments, linkers L and L′ are each independently selected from a bond, —C(O)—, —C(O)O—, —C(O)NH—, substituted or unsubstituted C₁₋₂₀ alkylene, substituted or unsubstituted —C(O)—C₁₋₂₀ alkylene, substituted or unsubstituted —C(O)O—C₁₋₂₀ alkylene, substituted or unsubstituted —C(O)NH—C₁₋₂₀ alkylene, and substituted or unsubstituted 2 to 20 membered heteroalkylene; wherein the substituted C₁₋₂₀ alkylene, substituted —C(O)—C₁₋₂₀ alkylene, substituted —C(O)O—C₁-20 alkylene, substituted —C(O)NH—C₁-20 alkylene, and substituted 2 to 20 membered heteroalkylene groups can be substituted with C₁₋₁₀ alkyl, 2 to 12 membered heteroalkyl, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, cycloalkyl, heterocycloalkyl, aryl, or heteroaryl, wherein each R′, R″, R′″ and R″″ groups are hydrogen, C₁₋₁₀ alkyl, 2 to 12 membered heteroalkyl, cycloalkyl, heterocycloalkyl, aryl, or arylalkyl groups.

In some embodiments, linkers L and L′ are each independently selected from a bond, —C(O)—, —C(O)O—, —C(O)NH—, substituted or unsubstituted C₁₋₈ alkylene, substituted or unsubstituted —C(O)—C₁₋₈ alkylene, substituted or unsubstituted —C(O)O—C₁₋ₛ alkylene, substituted or unsubstituted —C(O)NH—C₁₋₈ alkylene, and substituted or unsubstituted 2 to 10 membered heteroalkylene; wherein the substituted C₁₋₈ alkylene, substituted —C(O)—C₁₋₈ alkylene, substituted —C(O)O—C₁₋₈ alkylene, substituted —C(O)NH—C₁₋₈ alkylene, and substituted 2 to 10 membered heteroalkylene groups can be substituted with C₁₋₆ alkyl, 2 to 8 membered heteroalkyl, —OR′, ═O, —NR′R″, —SR′, -halogen, —OC(O)R′, —C(O)R′, —CO₂R′, —CONR′R″, —OC(O)NR′R″, —NR″C(O)R′, —NR″C(O)₂R′, —CN, —NO₂, cycloalkyl, heterocycloalkyl, aryl, or heteroaryl, wherein each R′ and R″ groups are hydrogen, C₁₋₆ alkyl, 2 to 8 membered heteroalkyl, C₃₋₈ cycloalkyl, 3 to 8 membered heterocycloalkyl, phenyl, benzyl, phenethyl, pyridylmethyl, or phenoxymethyl.

In some embodiments, linkers L and L′ are each independently selected from a bond, —C(O)—, —C(O)O—, —C(O)NH—, substituted or unsubstituted C₁₋₆ alkylene, substituted or unsubstituted —C(O)—C₁₋₆ alkylene, substituted or unsubstituted —C(O)O—C₁₋₆ alkylene, substituted or unsubstituted —C(O)NH—C₁₋₆ alkylene, and substituted or unsubstituted 2 to 8 membered heteroalkylene; wherein the substituted C₁₋₆ alkylene, substituted —C(O)—C₁₋₆ alkylene, substituted —C(O)O—C₁₋₆ alkylene, substituted —C(O)NH—C₁₋₆ alkylene, and substituted 2 to 8 membered heteroalkylene groups can be substituted with at least one of the following substituents selected from —OH, —NH₂, —SH, —CN, —CF₃, —COOH, —C(O)NH₂, halogen, unsubstituted C₁₋₃ alkyl, and substituted C₁₋₃ alkyl.

The molecular weights of the PBA modified multi-arm PEG polymer backbone of formula (I) and the 1,2-diol modified multi-arm PEG polymer backbone of formula (II) can vary and will depend upon the selection and value of each subscript a, a′, b, b′, c, c′, d, d′, m₁ and m₂; the identity of each linker L and L′; and the identity of each phenylboronic acid derivative and each 1,2-diol moiety (described in further detail below). In some embodiments, the PBA modified multi-arm PEG polymer backbone of formula (I) and the 1,2-diol modified multi-arm PEG polymer backbone of formula (II) can have molecular weights ranging from about 1.0 kDa to about 5,000 kDa. In some embodiments, the molecular weights of the PBA modified multi-arm PEG polymer backbone of formula (I) and the 1,2-diol modified multi-arm PEG polymer backbone of formula (II) are independently about 1.5 kDa, or about 2.0, 2.5, 3.0, 3.5, 4.0, 4.5, 5.0, 5.5, 6.0, 7.0, 8.0, 9.0, 10, 12, 14, 16, 18, 20, 22, 24, 26, 28, 30, 32, 35, 40, 50, 75, 100, 125, 150, 175, 200, 250, 300, 400, 500, 600, 700, 800, 900, 1,000, 2,000, 3,000, 4,000, or about 5,000 kDa. In some embodiments, the molecular weights of the PBA-PEG polymer backbone of formula (I) and the 1,2-diol-PEG polymer backbone of formula (II) independently range from about 1.5 kDa to about 3,000 kDa, or from about 2.0 to about 2,000, 2.5 to 1,000, 3.0 to 900, 3.5 to 800, 4.0 to 700, 4.5 to 600, 5.0 to 500, 5.5 to 400, 6.0 to 300, 7.0 to 250, 8.0 to 200, 9.0 to 175, 10 to 150, 12 to 125, 14 to 100, 16 to 75, 18 to 50, 20 to 40, 22 to 35, or from about 24 kDa to about 32 kDa.

In some embodiments, the PBA modified multi-arm PEG polymer backbone of formula (I) has a molecular weight ranging from about 1.0 kDa to about 100 kDa. In some embodiments, the PBA-PEG polymer backbone of formula (I) has a molecular weight of about 1.5 kDa, or 2.0 kDa, 2.5, 3.0, 3.5, 4.0, 4.5, 5.0, 6.0, 7.0, 8.0, 9.0, 10.0, 12.0, 13.0, 14.0, 15.0, 16.0, 17.0, 18.0, 19.0, 20.0, 22.0, 25.0, 27.0, 30.0, 35.0, 40.0, 50.0, 60.0, 70.0, 80.0, 90.0 or about 100 kDa. In some embodiments, the PBA-PEG polymer backbone of formula (I) has a molecular weight ranging from about 1.5 kDa to about 75.0 kDa, or about 2.0 to about 70.0, 2.5 to 65.0, 3.0 to 60.0, 3.5 to 55.0, 4.0 to 50.0, 4.5 to 45.0, 5.0 to 40.0, 6.0 to 35.0, 7.0 to 30.0, 8.0 to 27.0, 9.0 to 25.0, 10.0 to 22.0, 12.0 to 20.0, 13.0 to 19.0, 14.0 to 18.0, or from about 15.0 kDa to about 17.0 kDa. In some embodiments, the PBA-PEG polymer backbone of formula (I) has a molecular weight ranging from about 6.0 kDa to about 30.0 kDa. In some embodiments, the PBA-PEG polymer backbone of formula (I) has a molecular weight ranging from about 7.0 kDa to about 25.0 kDa. In some embodiments, the PBA-PEG polymer backbone of formula (I) has a molecular weight ranging from about 8.5 kDa to about 20.0 kDa. In some embodiments, the PBA-PEG polymer backbone of formula (I) has a molecular weight ranging from about 8.0 kDa to about 12.0 kDa.

In some embodiments, the 1,2-diol modified multi-arm PEG polymer backbone of formula (II) has a molecular weight ranging from about 1.5 kDa to about 125 kDa. In some embodiments, the 1,2-diol-PEG polymer backbone of formula (II) has a molecular weight ranging from about 2.0 kDa, or 2.5 kDa, 3.0, 3.5, 4.0, 4.5, 5.0, 6.0, 7.0, 8.0, 9.0, 10.0, 12.0, 13.0, 14.0, 15.0, 16.0, 17.0, 18.0, 19.0, 20.0, 22.0, 25.0, 27.0, 30.0, 35.0, 40.0, 50.0, 60.0, 70.0, 80.0, 90.0, 100, or about 110 kDa. In some embodiments, the 1,2-diol-PEG polymer backbone of formula (II) has a molecular weight ranging from about 2.0 kDa to about 120 kDa, or about 2.5 to about 115, 3.0 to 110, 3.5 to 100, 4.0 to 80.0, 4.5 to 75.0, 5.0 to 70.0, 6.0 to 65.0, 7.0 to 55.0, 8.0 to 45.0, 9.0 to 40.0, 10.0 to 45.0, 12.0 to 35.0, 13.0 to 30.0, 14.0 to 27.0, 15.0 to 25.0, or from about 16.0 kDa to about 22.0 kDa. In some embodiments, the 1,2-diol-PEG polymer backbone of formula (II) has a molecular weight ranging from about 7.0 kDa to about 35.0 kDa. In some embodiments, the 1,2-diol-PEG polymer backbone of formula (II) has a molecular weight ranging from about 7.5 kDa to about 20.0 kDa. In some embodiments, the 1,2-diol-PEG polymer backbone of formula (II) has a molecular weight ranging from about 8.0 kDa to about 18.0 kDa. In some embodiments, the 1,2-diol-PEG polymer backbone of formula (II) has a molecular weight ranging from about 8.5 kDa to about 12.5 kDa.

In some embodiments, the dynamic polymeric hydrogel composition comprises a combination of a PBA modified multi-arm PEG polymer backbone of formula (I) and a 1,2-diol modified multi-arm PEG polymer backbone of formula (II) wherein subscripts a, a′, b, b′, c, c′, d, and d′ are each independently an integer selected from 1 to 500; subscripts m₁ and m₂ are each independently an integer selected from 10 to 20,000; linkers L and L′ are each independently selected from a bond, —C(O)—, substituted or unsubstituted alkylene, substituted or unsubstituted —C(O)-alkylene, and substituted or unsubstituted heteroalkylene; each J is a phenylboronic acid derivative; and each Q is a 1,2-diol moiety. The PBA modified multi-arm PEG polymer backbone and the 1,2-diol modified multi-arm PEG polymer backbone are reversibly covalently cross-linked through the phenylboronic acid derivatives and the 1,2-diol groups.

In some embodiments, the dynamic polymeric hydrogel composition comprises a combination of a PBA modified multi-arm PEG polymer backbone of formula (I) and a 1,2-diol modified multi-arm PEG polymer backbone of formula (II) wherein subscripts a, a′, b, b′, c, c′, d, and d′ are each independently an integer selected from 10 to 250; subscripts m₁ and m₂ are each independently an integer selected from 25 to 10,000; each linker L is selected from a bond, —C(O)—, substituted or unsubstituted C₁₋₆ alkylene, and unsubstituted —C(O)—C₁₋₆ alkylene, wherein substituted C₁₋₆ alkylene is substituted with at least one substituent selected from —OH, —NH₂, —SH, —CN, —CF₃, —COOH, —C(O)NH₂, halogen, unsubstituted C₁₋₃ alkyl, and substituted C₁₋₃ alkyl; and each linker L′ is selected from a bond, —C(O)—, unsubstituted C₁₋₆ alkylene, and unsubstituted —C(O)—C₁₋₆ alkylene.

The PBA modified multi-arm PEG polymer backbone of formula (I) and 1,2-diol modified multi-arm PEG polymer backbone of formula (II) can be obtained commercially or readily synthesized using techniques generally known to those of skill in the art. For example, the starting materials and reagents used in preparing the disclosed PBA-PEG polymer backbone of formula (I) and the 1,2-diol-PEG polymer backbone of formula (II) and compositions thereof are either available from commercial suppliers such as Aldrich Chemical Co. (Milwaukee, Wis.), Acros Organics (Morris Plains, N.J.), Fisher Scientific (Pittsburgh, Pa.), Polysciences Inc. (Warrington, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Phenylboronic Acid Derivatives

As described above, the phenylboronic acid derivatives are attached to the terminal ends of each arm of the polymer backbone structure through a linker. Any suitable phenylboronic acid (PBA) derivative is useful in the dynamic polymeric hydrogel compositions of the present invention. In the context of the present invention, a PBA derivative is a type of arylboronic acid moiety. In other words, the PBA derivative is any chemical compound or fragment thereof that contains aryl group substituted with one or more —B(OH)₂ groups. In general, boronic acids are typically derived synthetically from primary sources of boron, such as boric acid. Dehydration of boric acid with alcohols gives rises to borate esters, which are precursors of boronic acids. Boronic acids can also be produced from the second oxidation of boranes.

More specifically, arylboronic acids can be produced from many different well-known synthetic approaches, such as, for example, electrophilic trapping of arylmetal intermediate with borates from aryl halides or from directed ortho-metalation; transmetalation of arylsilanes and arylstannanes; transition metal-catalyzed coupling between aryl halides and diboronyl reagents; direct boronation via transition metal-catalyzed aromatic C—H functionalization; alkynylboronate cycloaddition and aromatization. A discussion of the various methods of preparation and properties of many numerous arylboronic acid species, substituted with a variety of different substituents and functional groups can be found in “Boronic Acids.” Dennis Hall, Ed., Wiley-VCH Verlag, 2005, which is incorporated by reference herein at least for its teachings of arylboronic acid derivatives and their preparation. The PBA derivatives of the dynamic polymeric hydrogel compositions herein can be obtained commercially from known suppliers or readily synthesized using the methods and techniques introduced above.

In some embodiments, the PBA derivative of the PBA modified multi-arm PEG polymer backbone comprises a phenylboronic acid group of formula (III):

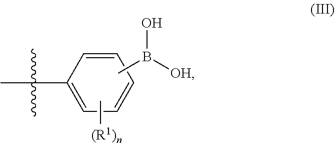

wherein each R¹ is each independently selected from the group consisting of substituted or unsubstituted alkyl, substituted or unsubstituted heteroalkyl, halogen, —CN, —OR^{a}, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —SR^{a}, —SiR^{a}R^{b}R^{c}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NRC(O)₂R^{a}, —NR—C(NR^{a}R^{b}R^{c})═NR^{d}, —NR—C(NR^{a}R^{b})═NR^{c}, —S(O)R^{a}, —S(O)₂R^{a}, —S(O)₂NR^{a}R^{b}, —NRSO₂R^{a}, wherein each R^{a}, R^{b}, R^{c} and R^{d} is independently selected from the group consisting of hydrogen, alkyl, heteroalkyl, cycloalkyl, heterocycloalkyl, aryl, and arylalkyl groups; and subscript n is an integer from 0-4; wherein the pKa of the phenylboronic acid group is less than 7.8.

In some embodiments, each R¹ of formula (III) is independently selected from the group consisting of substituted or unsubstituted C₁₋₂₀ alkyl, substituted or unsubstituted 2 to 20 membered heteroalkyl, halogen, —CN, —OR^{a}, —NO₂, substituted or unsubstituted C₃₋₁₀ cycloalkyl, substituted or unsubstituted 3 to 10 membered heterocycloalkyl, substituted or unsubstituted C₆₋₁₂ aryl, substituted or unsubstituted 5 to 12 membered heteroaryl, —NR^{a}R^{b}, —CH₂NR^{a}R^{b}, —SR^{a}, —SiR^{a}R^{b}R^{c}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NRC(O)₂R^{a}, —NR—C(NR^{a}R^{b}R^{c})═NR^{d}, —NR—C(NR^{a}R^{b})═NR^{c}, —S(O)R^{a}, —S(O)₂R^{a}, —S(O)₂NR^{a}R^{b}, and —NRSO₂R^{a}, wherein each R^{a}, R^{b}, R^{c} and R^{d} is independently selected from the group consisting of hydrogen, C₁₋₁₀ alkyl, 2 to 10 membered heteroalkyl, C₃₋₈ cycloalkyl, 3 to 8 membered heterocycloalkyl, aryl, phenyl, naphthyl, benzyl, phenethyl, pyridylmethyl, and phenoxymethyl; and wherein the substituted C₁₋₂₀ alkyl, substituted 2 to 20 membered heteroalkyl, substituted C₃₋₁₀ cycloalkyl, substituted 3 to 10 membered heterocycloalkyl, substituted C₆₋₁₂ aryl, and substituted 5 to 12 membered heteroaryl groups can be substituted with C₁₋₆ alkyl, 2 to 8 membered heteroalkyl, —OR′, ═O, ═NR′, ═N—OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —S(O)R′, —S(O)₂R′, —S(O)₂NR′R″, —NRSO₂R′, —CN, —NO₂, C₃₋₁₀ cycloalkyl, 3 to 10 membered heterocycloalkyl, C₆₋₁₂ aryl, or 5 to 12 membered heteroaryl, wherein each R′, R″, and R′″ groups are hydrogen, C₁₋₆ alkyl, 2 to 6 membered heteroalkyl, C₃₋₈ cycloalkyl, 3 to 8 membered heterocycloalkyl, phenyl, benzyl, phenethyl, pyridylmethyl, or phenoxymethyl groups.

In some embodiments, each R¹ of formula (III) is independently selected from the group consisting of substituted or unsubstituted C₁₋₁₀ alkyl, substituted or unsubstituted 2 to 12 membered heteroalkyl, halogen, —CN, —OR^{a}, —NO₂, substituted or unsubstituted C₃₋₈ cycloalkyl, substituted or unsubstituted 3 to 8 membered heterocycloalkyl, substituted or unsubstituted C₆₋₁₀ aryl, substituted or unsubstituted 5 to 10 membered heteroaryl, —NR^{a}R^{b}, —CH₂NR^{a}R^{b}, —SR^{a}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NR^{b}C(O)₂R^{a}, —S(O)R^{a}, —S(O)₂R^{a}, —S(O)₂NR^{a}R^{b}, and —NRSO₂R^{a}, wherein each R^{a}, R^{b}, and R^{c} is independently selected from the group consisting of hydrogen, C₁₋₈ alkyl, 2 to 8 membered heteroalkyl, C₃₋₆ cycloalkyl, 3 to 6 membered heterocycloalkyl, phenyl, benzyl, phenethyl, pyridylmethyl, and phenoxymethyl; and wherein the substituted C₁₋₁₀ alkyl, substituted 2 to 12 membered heteroalkyl, substituted C₃₋₈ cycloalkyl, substituted 3 to 8 membered heterocycloalkyl, substituted C₆₋₁₀ aryl, and substituted 5 to 10 membered heteroaryl groups can be substituted with C₁₋₆ alkyl, 2 to 8 membered heteroalkyl, —OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —S(O)R′, —S(O)₂R′, —CN, —NO₂, C₃₋₆ cycloalkyl, 3 to 6 membered heterocycloalkyl, phenyl, or benzyl, wherein each R′, R″, and R′″ groups are hydrogen, C₁₋₃ alkyl, 2 to 4 membered heteroalkyl, C₃₋₆ cycloalkyl, 3 to 6 membered heterocycloalkyl, phenyl, or benzyl groups.

In some embodiments, each R¹ of formula (III) is independently selected from the group consisting of substituted or unsubstituted C₁₋₈ alkyl, substituted or unsubstituted 2 to 10 membered heteroalkyl, halogen, —CN, —OR^{a}, —NO₂, substituted or unsubstituted C₃₋₆ cycloalkyl, substituted or unsubstituted 3 to 6 membered heterocycloalkyl, substituted or unsubstituted phenyl, substituted or unsubstituted 5 to 10 membered heteroaryl, —NR^{a}R^{b}, —CH₂NR^{a}R^{b}, —SR^{a}, —OC(O)R^{a}, —C(O)R^{a}, —CO₂R^{a}, —CONR^{a}R^{b}, —OC(O)NR^{a}R^{b}, —NR^{b}C(O)R^{a}, —S(O)R^{a}, and —S(O)₂R^{a}, wherein each R^{a} and R^{b} is independently selected from the group consisting of hydrogen, C₁₋₆ alkyl, 2 to 6 membered heteroalkyl, C₃-s cycloalkyl, 3 to 6 membered heterocycloalkyl, phenyl, and benzyl; and wherein the substituted C₁₋ₛ alkyl, substituted 2 to 10 membered heteroalkyl, substituted C₃₋₆ cycloalkyl, substituted 3 to 6 membered heterocycloalkyl, substituted phenyl, and substituted 5 to 10 membered heteroaryl groups can be substituted with C₁₋₃ alkyl, 2 to 4 membered heteroalkyl, —OH, —OCH₃, —OCH₂CH₃, —OCH(CH₃)₂, —NH₂, —N(CH₃)₂, —CF₃, —CO₂H, —CH₂CO₂H, —CH₂CONH₂, —COOCH₃, —COCH₃, —CONH₂, —CON(CH₃)₂, —OCF₃, —NHCOCH₃, -halogen, —CN, —NO₂, cyclopentyl, cyclohexyl, phenyl, or benzyl.

In some embodiments, each R¹ of formula (III) is independently selected from the group consisting of substituted or unsubstituted C₁₋₆ alkyl, C₁₋₆ alkoxy, C₁₋₆ haloalkoxy, halogen, —CN, —OR^{a}, —NO₂, substituted or unsubstituted C₃₋₆ cycloalkyl, substituted or unsubstituted 3 to 6 membered heterocycloalkyl, substituted or unsubstituted phenyl, substituted or unsubstituted 5 to 6 membered heteroaryl, —NR^{a}R^{b}, —CH₂NR^{a}R^{b}, —SR^{a}, —C(O)R^{a}, —CO₂R^{a}, —CONR^{a}R^{b}, —S(O)R^{a}, and —S(O)₂R^{a}, wherein each R^{a} and R^{b} is independently selected from the group consisting of hydrogen, C₁₋₆ alkyl, 2 to 6 membered heteroalkyl, C₃₋₆ cycloalkyl, 3 to 6 membered heterocycloalkyl, phenyl, and benzyl; and wherein the substituted C₁₋₆ alkyl, substituted C₃₋₆ cycloalkyl, substituted 3 to 6 membered heterocycloalkyl, substituted phenyl, and substituted 5 to 6 membered heteroaryl groups can be substituted with C₁₋₃ alkyl, —OH, —OCH₃, —NH₂, —N(CH₃)₂, —CF₃, —CO₂H, —CH₂CO₂H, —CH₂CONH₂, —COOCH₃, —COCH₃, —CON H₂, —CON(CH₃)₂, —OCF₃, -halogen, —CN, or —NO₂.

In some embodiments, subscript n of formula (III) is an integer selected from 0, 1, 2, 3, or 4. In some embodiments, subscript n of formula (III) is an integer selected from 0-3. In some embodiments, subscript n is an integer selected from 0-2. In some embodiments, subscript n is an integer selected from 0-1. In some embodiments, subscript n is 4. In some embodiments, subscript n is 3. In some embodiments, subscript n is 2. In some embodiments, subscript n is 1. In some embodiments, subscript n is 0.

In some embodiments, the phenylboronic acid group of the PBA derivative of formula (III) has a pKa that is less than 7.8 and greater than 0. In some embodiments, the phenylboronic acid group of the PBA derivative of formula (III) has a pKa of between about 1.0 and about 7.6. In some embodiments, the phenylboronic acid group has a pKa of about 1.5, 1.8, 2.0, 2.5, 3.0, 3.5, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, or about 7.4. In some embodiments, the PBA group has a pKa of between about 1.5 and about 7.4, 1.8 and 7.2, 2.0 and 7.0, 2.5 and 6.8, 3.0 and 6.6, 3.5 and 6.4, 3.8 and 6.2, 4.0 and 6.0, 4.2 and 5.8, 4.4 and 5.6, or between about 4.6 and about 5.4. In some embodiments, the PBA group has a pKa of between about 3.5 and about 7.4. In some embodiments, the PBA group has a pKa of between about 4.0 and about 7.2. In some embodiments, the PBA group has a pKa of between about 4.0 and about 6.8. In some embodiments, the PBA group has a pKa of between about 4.0 and about 6.6.

In some embodiments, the PBA derivative of the PBA modified multi-arm PEG polymer backbone comprises a phenylboronic acid group of formula (III) wherein each R¹ is each independently selected from the group consisting of substituted or unsubstituted alkyl, substituted or unsubstituted heteroalkyl, halogen, —CN, —OH, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; R^{a} and R^{b} are each independently selected from the group consisting of hydrogen and C₁₋₆ alkyl; and subscript n is an integer from 0-4; wherein the pKa of the phenylboronic acid group is less than 7.8.

In some embodiments, the PBA derivative of the PBA modified multi-arm PEG polymer backbone comprises a phenylboronic acid group of formula (III) wherein each R¹ is each independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b}; R^{a} and R^{b} are each independently selected from the group consisting of hydrogen and C₁₋₆ alkyl; and subscript n is an integer from 0-3; wherein the pKa of the phenylboronic acid group is between about 3.5 and about 7.4. In some embodiments, each R¹ of formula (III) is independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b}, wherein R^{a} and R^{b} are each independently selected from the group consisting of hydrogen and C₁₋₆ alkyl; and wherein the substituted C₁₋₆ alkyl and substituted phenyl can be substituted with C₁₋₃ alkyl, -halogen, —OH, —OCH₃, —NH₂, —N(CH₃)₂, —CF₃, —CO₂H, —CH₂CO₂H, —CH₂CONH₂, —COOCH₃, —COCH₃, —CONH₂, —CON(CH₃)₂, —OCF₃, —CN, —NO₂, or phenyl.

In some embodiments, the PBA derivative of the PBA modified multi-arm PEG polymer backbone comprises a phenylboronic acid group of formula (IIIA) or formula (IIIB).

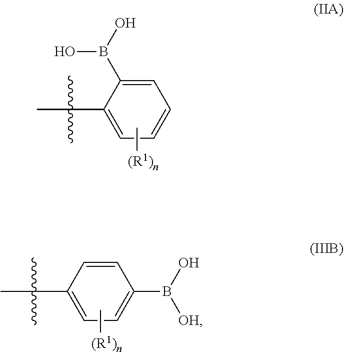

wherein each R¹ is each independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NR^{a}R^{b}, —CH₂NR^{a}R^{b}, —C(O)R^{a}, and —C(O)OR^{a}; R^{a} and R^{b} are each independently selected from the group consisting of hydrogen and C₁₋₃ alkyl; subscript n is an integer from 0-2; and wherein the pKa of the phenylboronic acid group is between about 4.0 and about 7.2. In some embodiments, each R¹ of formula (IIIA) or formula (IIIB) is independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NH₂, —N(CH₃)₂, —CO₂H, —COOCH₃, —COCH₃, and —OCF₃, wherein the substituted C₁₋₃ alkyl group can be substituted with —OH, —OCH₃, —NH₂, —N(CH₃)₂, —CF₃, —CO₂H, —CH₂CO₂H, —CH₂CONH₂, —COOCH₃, —COCH₃, —CONH₂, —CON(CH₃)₂, —CN, or —NO₂; subscript n is either 0, 1, or 2; and wherein the pKa of the phenylboronic acid group is between about 4.0 and about 7.2. In some embodiments, each R¹ of formula (IIIA) or formula (IIIB) is independently selected from the group consisting of trifluoromethyl, methoxy, ethoxy, fluoro, chloro, bromo, iodo, —CN, —OH, —NO₂, phenyl, benzyl, —NH₂, —N(CH₃)₂, —CH₂NH₂, —CH₂N(CH₃)₂, —CO₂H, —COOCH₃, —COCH₃, and —OCF₃; subscript n is either 0, 1, or 2; and wherein the pKa of the phenylboronic acid group is between about 4.0 and about 7.2.

In some embodiments, the PBA derivative of the PBA modified multi-arm PEG polymer backbone comprises a phenylboronic acid group of formula (IIIA) selected from the group consisting of:

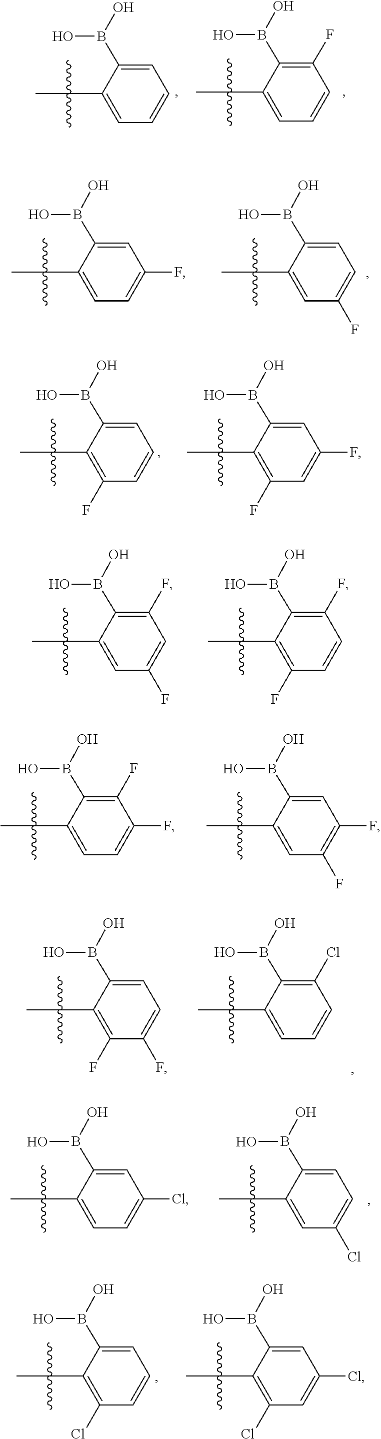

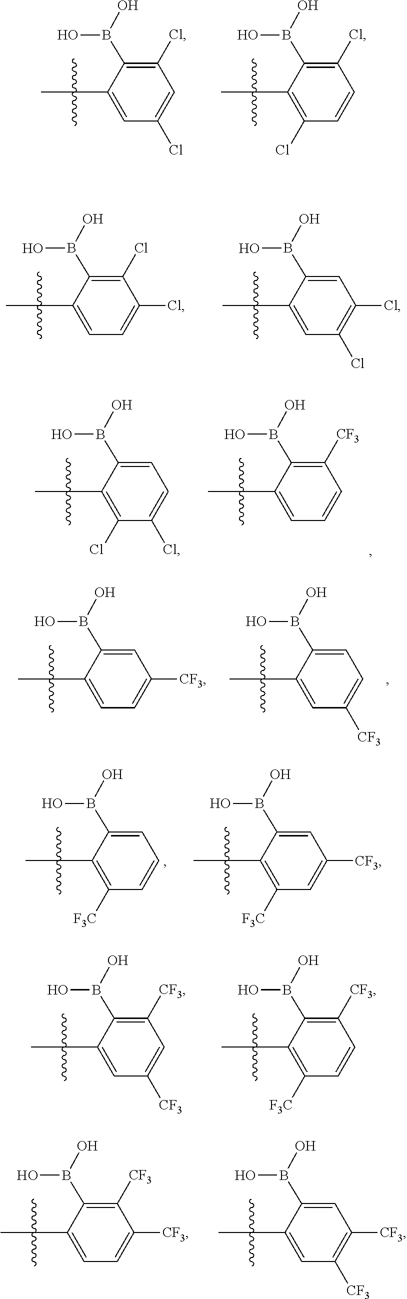

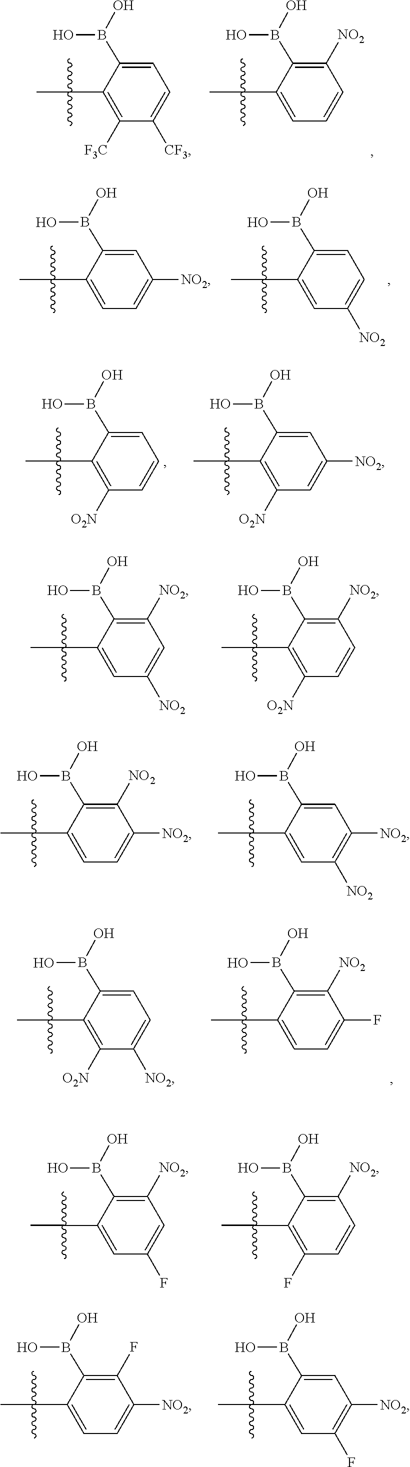

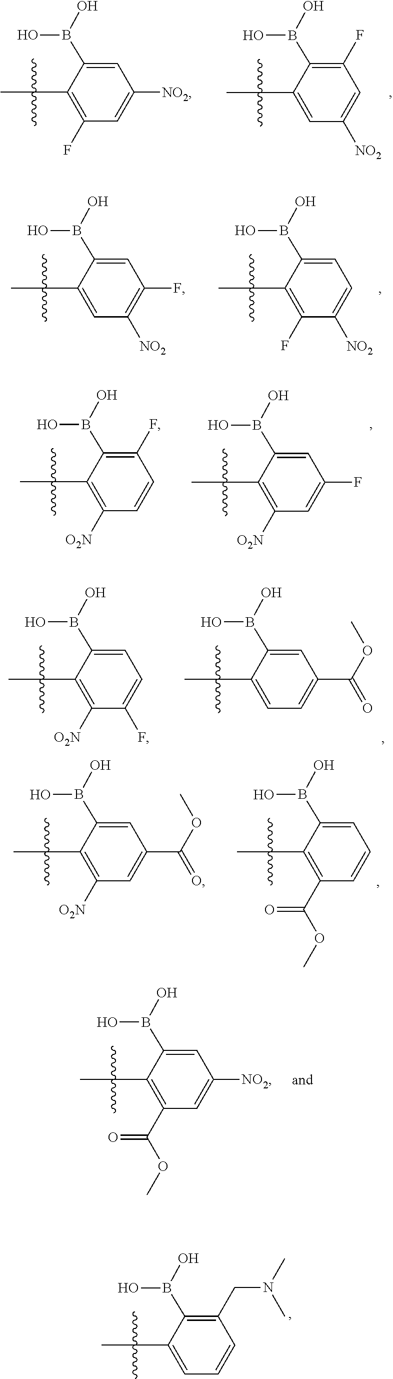

wherein the pKa of the phenylboronic acid group is between about 4.0 and about 7.2.

In some embodiments, the PBA derivative of the PBA modified multi-arm PEG polymer backbone comprises a phenylboronic acid group of formula (IIIA) selected from the group consisting of:

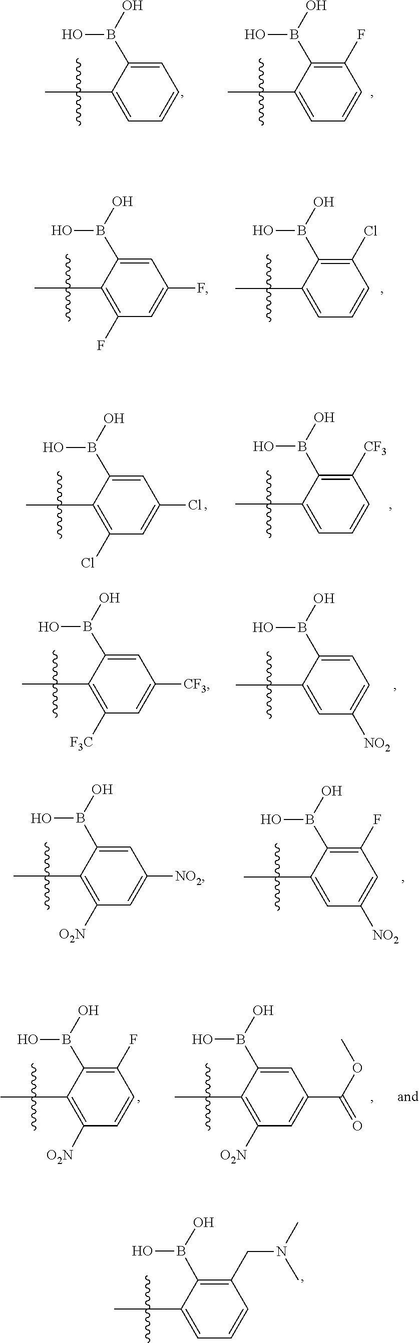

wherein the pKa of the phenylboronic acid group is between about 4.0 and about 7.2.

In some embodiments, the PBA derivative of the PBA modified multi-arm PEG polymer backbone comprises a phenylboronic acid group of formula (IIIB) selected from the group consisting of:

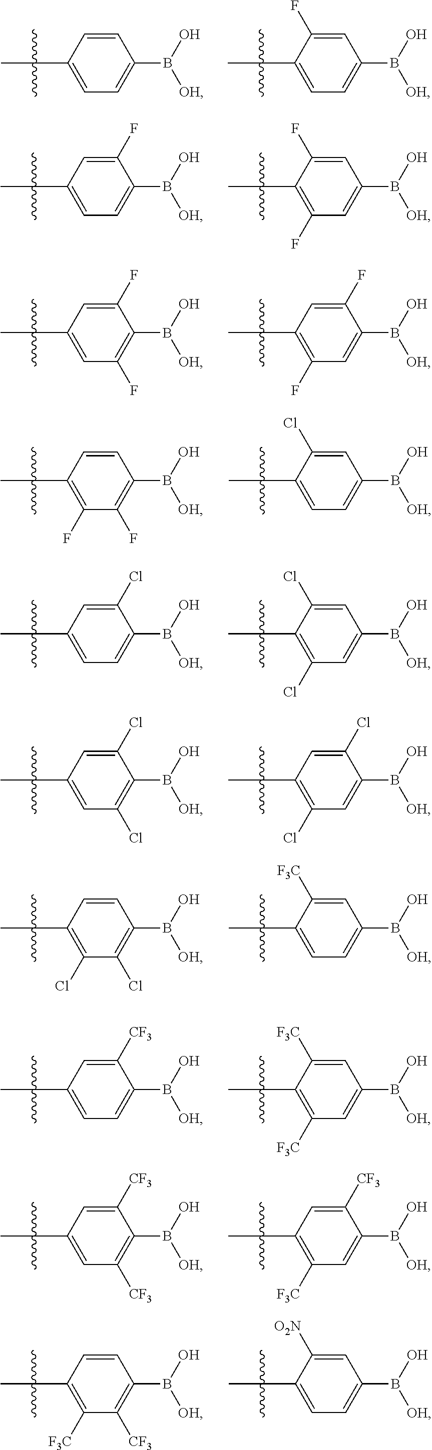

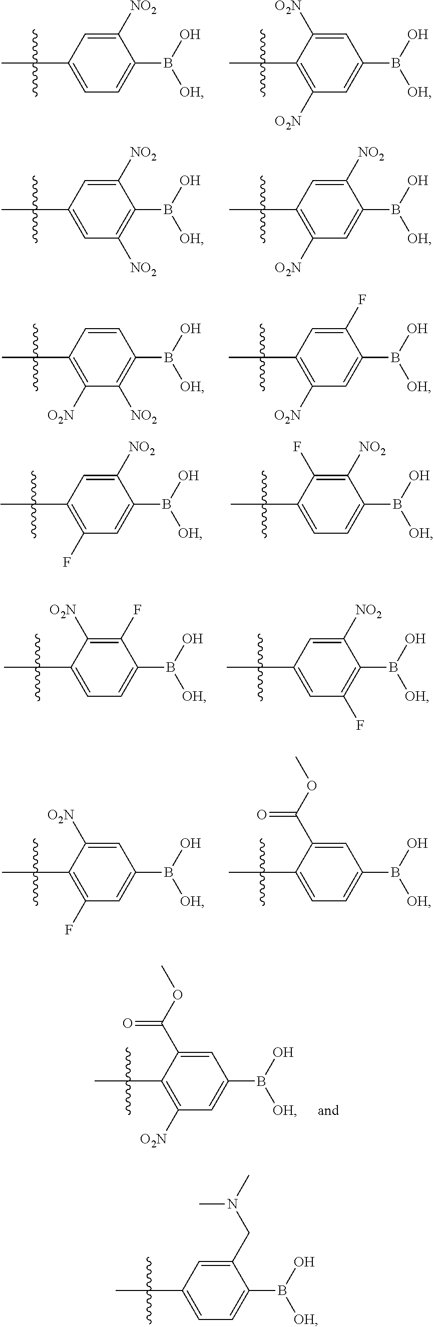

wherein the pKa of the phenylboronic acid group is between about 4.0 and about 7.2.

In some embodiments, the PBA derivative of the PBA modified multi-arm PEG polymer backbone comprises a phenylboronic acid group of formula (IIIB) selected from the group consisting of:

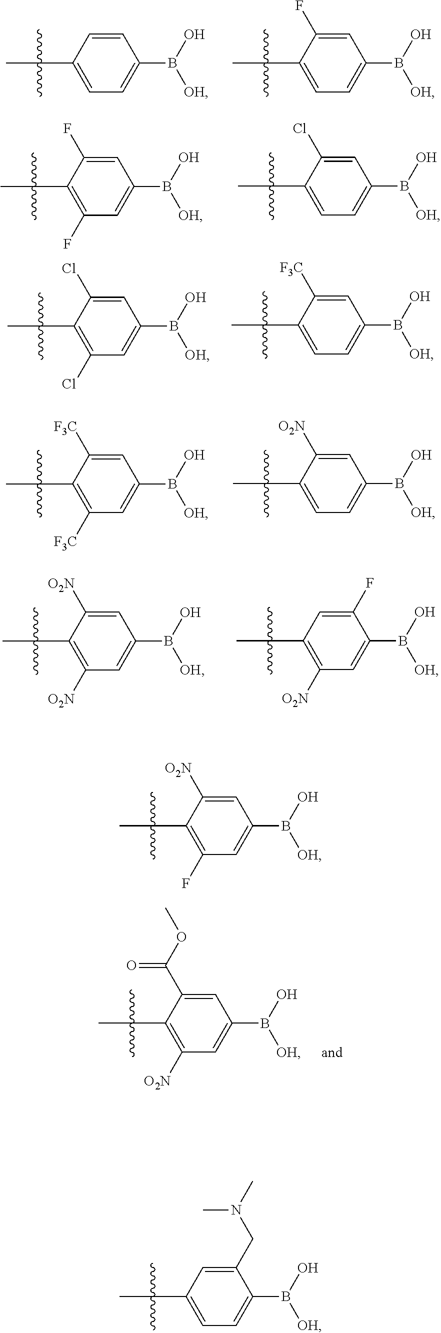

wherein the pKa of the phenylboronic acid group is between about 4.0 and about 7.2.

### 1,2-Diol Moieties

The 1,2-diol moieties are attached to the terminal ends of each arm of the polymer backbone structure through a linker. Any suitable 1,2-diol moiety is useful in the dynamic polymeric hydrogel compositions of the present invention. In the context of the present invention, a 1,2-diol moiety is a type of dihydroxy alcohol chemical entity containing two hydroxyl groups connected to adjacent carbon atoms of a hydrocarbon group. Thus, the 1,2-diol moiety is any chemical compound or fragment thereof that contains one or more vicinal diol groups. In some embodiments, the 1,2-diol moiety is a 1,2-cis-diol moiety. In some embodiments, the 1,2-diol moiety is a 1,2-trans-diol moiety. In general, 1,2-diols are typically derived from many different well-known synthetic approaches, such as, for example, the hydrogenation of a hydroformylated fatty acid, the hydrogenation of an epoxidized fatty acid or epoxidized fatty acid alcohol, or the reduction of an α,ω-dicarboxylic acid. The 1,2-diol moieties of the dynamic polymeric hydrogel compositions herein can be obtained commercially from known suppliers or readily synthesized using the methods introduced above.

In some embodiments, the 1,2-diol-moiety of the 1,2-diol modified multi-arm PEG polymer backbone comprises a 1,2-cis-diol group of formula (IV):

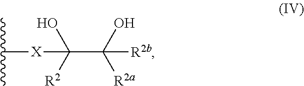

wherein R², R^{2a}, and R^{2b} are each independently selected from the group consisting of hydrogen, substituted or unsubstituted alkyl, substituted or unsubstituted heteroalkyl, halogen, —CN, —OR^{a}, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NR^{b}C(O)₂R^{a} —NR—C(NR^{a}R^{b}R^{c})═NR^{d}, —NR—C(NR^{a}R^{b})═NR, —SR^{a}, —S(O)R^{a}, —S(O)₂R^{a}, —S(O)₂NR^{a}R^{b}, and —NRSO₂R^{a}; wherein, optionally, R² and one of R²a or R^{2b} are combined together to form a substituted or unsubstituted cycloalkyl, substituted or unsubstituted heterocycloalkyl, substituted or unsubstituted aryl, or substituted or unsubstituted heteroaryl; R^{a}, R^{b}, R^{c} and R^{d} are each independently selected from the group consisting of hydrogen, alkyl, heteroalkyl, cycloalkyl, heterocycloalkyl, aryl, and arylalkyl groups; and X is selected from the group consisting of a bond, —C(O)—, —C(O)O—, —C(O)NH—, substituted or unsubstituted alkylene, substituted or unsubstituted —C(O)-alkylene, substituted or unsubstituted —C(O)O-alkylene, substituted or unsubstituted —C(O)NH-alkylene, and substituted or unsubstituted heteroalkylene.

In some embodiments, R², R²a, and R^{2b} of formula (IV) are each independently selected from the group consisting of hydrogen, substituted or unsubstituted C₁₋₂₀ alkyl, substituted or unsubstituted 2 to 20 membered heteroalkyl, halogen, —CN, —OR^{a}, substituted or unsubstituted C₃₋₁₀ cycloalkyl, substituted or unsubstituted 3 to 10 membered heterocycloalkyl, substituted or unsubstituted C₆₋₁₂ aryl, substituted or unsubstituted 5 to 12 membered heteroaryl, —NR^{a}R^{b}, —CH₂NR^{a}R^{b}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NR^{b}C(O)₂R^{a}, —NR—C(NR^{a}R^{b}R^{c})═NR^{d}, —NR—C(NR^{a}R^{b})═NR^{c}, —SR^{a}, —S(O)R^{a}, —S(O)₂R^{a}, —S(O)₂NR^{a}R^{b}, and —NRSO₂R^{a}; wherein, optionally, R² and one of R²a or R^{2b} are combined together to form a substituted or unsubstituted C₃₋₁₀ cycloalkyl, substituted or unsubstituted 3 to 10 membered heterocycloalkyl, substituted or unsubstituted C₆₋₁₂ aryl, or substituted or unsubstituted 5 to 12 membered heteroaryl; wherein each R^{a}, R^{b}, R and R^{d} is independently selected from the group consisting of hydrogen, C₁₋₁₀ alkyl, 2 to 10 membered heteroalkyl, C₃₋₈ cycloalkyl, 3 to 8 membered heterocycloalkyl, aryl, phenyl, naphthyl, benzyl, phenethyl, pyridylmethyl, and phenoxymethyl; and wherein the substituted C₁₋₂₀ alkyl, substituted 2 to 20 membered heteroalkyl, substituted C₃₋₁₀ cycloalkyl, substituted 3 to 10 membered heterocycloalkyl, substituted C₆₋₁₂ aryl, and substituted 5 to 12 membered heteroaryl groups can be substituted with C₁₋₆ alkyl, 2 to 8 membered heteroalkyl, —OR′, ═O, ═NR′, ═N—OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —S(O)R′, —S(O)₂R′, —S(O)₂NR′R″, —NRSO₂R′, —CN, —NO₂, C₃₋₁₀ cycloalkyl, 3 to 10 membered heterocycloalkyl, C₆₋₁₂ aryl, or 5 to 12 membered heteroaryl, wherein each R′, R″, and R′″ groups are hydrogen, C₁₋₆ alkyl, 2 to 6 membered heteroalkyl, C₃₋₈ cycloalkyl, 3 to 8 membered heterocycloalkyl, phenyl, benzyl, phenethyl, pyridylmethyl, or phenoxymethyl groups.

In some embodiments, R², R²a, and R^{2b} of formula (IV) are each independently selected from the group consisting of hydrogen, substituted or unsubstituted C₁₋₁₀ alkyl, substituted or unsubstituted 2 to 12 membered heteroalkyl, halogen, —CN, —OR^{a}, substituted or unsubstituted C₃₋₈ cycloalkyl, substituted or unsubstituted 3 to 8 membered heterocycloalkyl, substituted or unsubstituted C₆₋₁₀ aryl, substituted or unsubstituted 5 to 10 membered heteroaryl, —NR^{a}R^{b}, —CH₂NR^{a}R^{b}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{c}, —NRC(O)₂R^{a}, —SR^{a}, —S(O)R^{a}, —S(O)₂R^{a}, —S(O)₂NR^{a}R^{b}, and —NRSO₂R^{a}; wherein, optionally, R² and one of R²a or R^{2b} are combined together to form a substituted or unsubstituted C₃₋₈ cycloalkyl, substituted or unsubstituted 3 to 8 membered heterocycloalkyl, substituted or unsubstituted C₆₋₁₀ aryl, or substituted or unsubstituted 5 to 10 membered heteroaryl; wherein each R^{a}, R^{b}, and R^{c} is independently selected from the group consisting of hydrogen, C₁₋₈ alkyl, 2 to 8 membered heteroalkyl, C₃₋₆ cycloalkyl, 3 to 6 membered heterocycloalkyl, phenyl, benzyl, phenethyl, pyridylmethyl, and phenoxymethyl; and wherein the substituted C₁₋₁₀ alkyl, substituted 2 to 12 membered heteroalkyl, substituted C₃₋₈ cycloalkyl, substituted 3 to 8 membered heterocycloalkyl, substituted C₆₋₁₀ aryl, and substituted 5 to 10 membered heteroaryl groups can be substituted with C₁₋₆ alkyl, 2 to 8 membered heteroalkyl, —OR′, —NR′R″, —SR′, -halogen, —OC(O)R′, —C(O)R′, —CO₂R′, —CONR′R″, —OC(O)NR′R″, —NR″C(O)R′, —NR′—C(O)NR″R′″, —NR″C(O)₂R′, —S(O)R′, —S(O)₂R′, —CN, —NO₂, C₃₋₆ cycloalkyl, 3 to 6 membered heterocycloalkyl, phenyl, or benzyl, wherein each R′, R″, and R′″ groups are hydrogen, C₁₋₃ alkyl, 2 to 4 membered heteroalkyl, C₃₋₆ cycloalkyl, 3 to 6 membered heterocycloalkyl, phenyl, or benzyl groups.

In some embodiments, R², R^{2a}, and R^{2b} of formula (IV) are each independently selected from the group consisting of hydrogen, substituted or unsubstituted C₁₋₈ alkyl, substituted or unsubstituted 2 to 10 membered heteroalkyl, halogen, —CN, —OR, substituted or unsubstituted C₃₋₇ cycloalkyl, substituted or unsubstituted 3 to 7 membered heterocycloalkyl, substituted or unsubstituted phenyl, substituted or unsubstituted 5 to 6 membered heteroaryl, —NR^{a}R^{b}, —CH₂NR^{a}R^{b}, —OC(O)R^{a}, —C(O)R^{a}, —CO₂R^{a}, —CONR^{a}R^{b}, —OC(O)NR^{a}R^{b}, —NR^{b}C(O)R^{a}, —SR^{a}, —S(O)R^{a}, and —S(O)₂R^{a}; wherein, optionally, R² and one of R²a or R^{2b} are combined together to form a substituted or unsubstituted C₃₋₇ cycloalkyl, substituted or unsubstituted 3 to 7 membered heterocycloalkyl, substituted or unsubstituted phenyl, or substituted or unsubstituted 5 to 6 membered heteroaryl; wherein each R^{a} and R^{b} is independently selected from the group consisting of hydrogen, C₁₋₆ alkyl, 2 to 6 membered heteroalkyl, C₃₋₆ cycloalkyl, 3 to 6 membered heterocycloalkyl, phenyl, and benzyl; and wherein the substituted C₁₋₈ alkyl, substituted 2 to 10 membered heteroalkyl, substituted C₃₋₇ cycloalkyl, substituted 3 to 7 membered heterocycloalkyl, substituted phenyl, and substituted 5 to 6 membered heteroaryl groups can be substituted with C₁₋₃ alkyl, 2 to 4 membered heteroalkyl, —OH, —OCH₃, —OCH₂CH₃, —OCH(CH₃)₂, —NH₂, —N(CH₃)₂, —CF₃, —CO₂H, —CH₂CO₂H, —CH₂CONH₂, —COOC H₃, —COCH₃, —CONH₂, —CON(CH₃)₂, —OCF₃, —NHCOCH₃, -halogen, —CN, —NO₂, cyclopentyl, cyclohexyl, phenyl, or benzyl.

In some embodiments, R², R²a, and R^{2b} of formula (IV) are each independently selected from the group consisting of hydrogen, substituted or unsubstituted C₁₋₆ alkyl, C₁₋₆ alkoxy, C₁₋₆ haloalkoxy, halogen, —CN, —OR^{a}, substituted or unsubstituted C₃₋₆ cycloalkyl, substituted or unsubstituted 3 to 6 membered heterocycloalkyl, substituted or unsubstituted phenyl, substituted or unsubstituted 5 to 6 membered heteroaryl, —NR^{a}R^{b}, —CH₂NR^{a}R^{b}, —C(O)R^{a}, —CO₂R^{a}, —CONR^{a}R^{b}, —SR^{a}, —S(O)R^{a}, and —S(O)₂R^{a}; wherein, optionally, R² and one of R²a or R^{2b} are combined together to form a substituted or unsubstituted C₃₋₆ cycloalkyl, substituted or unsubstituted 3 to 6 membered heterocycloalkyl, substituted or unsubstituted phenyl, or substituted or unsubstituted 5 to 6 membered heteroaryl; wherein each R^{a} and R^{b} is independently selected from the group consisting of hydrogen, C₁₋₆ alkyl, 2 to 6 membered heteroalkyl, C₃₋₆ cycloalkyl, 3 to 6 membered heterocycloalkyl, phenyl, and benzyl; and wherein the substituted C₁₋₆ alkyl, substituted C₃₋₆ cycloalkyl, substituted 3 to 6 membered heterocycloalkyl, substituted phenyl, and substituted 5 to 6 membered heteroaryl groups can be substituted with C₁₋₃ alkyl, —OH, —OCH₃, —NH₂, —N(CH₃)₂, —CF₃, —CO₂H, —CH₂CO₂H, —CH₂CONH₂, —COOCH₃, —COCH₃, —CONH₂, —CON(CH₃)₂, —OCF₃, -halogen, —CN, or —NO₂.

In some embodiments, X of formula (IV) is selected from the group consisting of a bond, —C(O)—, —C(O)O—, —C(O)NH—, substituted or unsubstituted C₁₋₈ alkylene, substituted or unsubstituted —C(O)—C₁₋₈ alkylene, substituted or unsubstituted —C(O)O—C₁₋₈ alkylene, substituted or unsubstituted —OC(O)—C₁₋ₛ alkylene, substituted or unsubstituted —C(O)NH—C₁₋₈ alkylene, and substituted or unsubstituted 2 to 10 membered heteroalkylene; wherein the substituted C₁₋₈ alkylene, substituted —C(O)—C₁₋₈ alkylene, substituted —C(O)O—C₁₋₈ alkylene, substituted —OC(O)—C₁₋₈ alkylene, substituted —C(O)NH—C₁₋₈ alkylene, and substituted 2 to 10 membered heteroalkylene groups can be substituted with C₁₋₆ alkyl, 2 to 8 membered heteroalkyl, —OR′, ═O, —NR′R″, —SR′, -halogen, —OC(O)R′, —C(O)R′, —CO₂R′, —CONR′R″, —OC(O)NR′R″, —NR″C(O)R′, —NR″C(O)₂R′, —CN, —NO₂, cycloalkyl, heterocycloalkyl, aryl, or heteroaryl, wherein each R′ and R″ groups are hydrogen, C₁₋₆ alkyl, 2 to 8 membered heteroalkyl, C₃₋₈ cycloalkyl, 3 to 8 membered heterocycloalkyl, phenyl, benzyl, phenethyl, pyridylmethyl, or phenoxymethyl.

In some embodiments, X of formula (IV) is selected from the group consisting of a bond, —C(O)—, —C(O)O—, —C(O)NH—, substituted or unsubstituted C₁₋₆ alkylene, substituted or unsubstituted —C(O)—C₁₋₆ alkylene, substituted or unsubstituted —C(O)O—C₁₋₆ alkylene, substituted or unsubstituted —OC(O)—C₁₋₆ alkylene, substituted or unsubstituted —C(O)NH—C₁₋₆ alkylene, and substituted or unsubstituted 2 to 8 membered heteroalkylene; wherein the substituted C₁₋₆ alkylene, substituted —C(O)—C₁₋₆ alkylene, substituted —C(O)O—C₁₋₆ alkylene, substituted —OC(O)—C₁₋₆ alkylene, substituted —C(O)NH—C₁₋₆ alkylene, and substituted 2 to 8 membered heteroalkylene groups can be substituted with at least one of the following substituents selected from —OH, —NH₂, —SH, —CN, —CF₃, —COOH, —C(O)NH₂, halogen, unsubstituted C₁₋₃ alkyl, and substituted C₁₋₃ alkyl.

In some embodiments, X of formula (IV) is selected from the group consisting of a bond, substituted or unsubstituted C₁₋₆ alkylene, substituted or unsubstituted —C(O)—C₁₋₆ alkylene, substituted or unsubstituted —C(O)O—C₁₋₆ alkylene, substituted or unsubstituted —OC(O)—C₁₋₆ alkylene, substituted or unsubstituted —C(O)NH—C₁₋₆ alkylene, and substituted or unsubstituted 2 to 8 membered heteroalkylene; wherein the substituted C₁₋₆ alkylene, substituted —C(O)—C₁₋₆ alkylene, substituted —C(O)O—C₁₋₆ alkylene, substituted —OC(O)—C₁₋₆ alkylene, substituted —C(O)NH—C₁₋₆ alkylene, and substituted 2 to 8 membered heteroalkylene groups can be substituted with at least one of the following substituents selected from —OH, —NH₂, —SH, —CN, —CF₃, —COOH, —C(O)NH₂, halogen, unsubstituted C₁₋₃ alkyl, and substituted C₁₋₃ alkyl. In some embodiments, X of formula (IV) is selected from the group consisting of a bond, C₁₋₆ alkylene, C₁₋₆ alkoxy, —C(O)O—C₁₋₆ alkylene, and —OC(O)—C₁₋₆ alkylene; wherein, optionally, the C₁₋₆ alkylene, C₁₋₆ alkoxy, —C(O)O—C₁₋₆ alkylene, and —OC(O)—C₁₋₆ alkylene groups are each independently substituted with 1-4 substituents each independently selected from the group consisting of —OH, C₁₋₃ alkyl, —C(O)R^{a}, and —C(O)—. In some embodiments, X of formula (IV) is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—.

In some embodiments, the 1,2-diol moiety of the 1,2-diol modified multi-arm PEG polymer backbone comprises a 1,2-cis-diol group of formula (IV) wherein R², R^{2a}, and R^{2b} are each independently selected from the group consisting of hydrogen, substituted or unsubstituted alkyl, substituted or unsubstituted heteroalkyl, halogen, —CN, —OH, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a}; wherein, optionally, R² and one of R²a or R^{2b} are combined together to form a substituted or unsubstituted cycloalkyl, substituted or unsubstituted heterocycloalkyl, substituted or unsubstituted aryl, or substituted or unsubstituted heteroaryl; R^{a} and R^{b} are each independently selected from the group consisting of hydrogen and C₁₋₆ alkyl; and X is selected from the group consisting of a bond, substituted or unsubstituted alkylene, substituted or unsubstituted heteroalkylene, and —C(O)—.

In some embodiments, the 1,2-diol moiety of the 1,2-diol modified multi-arm PEG polymer backbone comprises a 1,2-cis-diol group of formula (IV), wherein R², R²a, and R^{2b} are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, —NR^{a}R^{b}, —CH₂NR^{a}R^{b}, —C(O)R^{a}, —CO₂R^{a}, and —C(O)NR^{a}R^{b}; wherein, optionally, R² and one of R²a or R^{2b} are combined together to form a substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₆ alkyl; and X is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—. In some embodiments, R², R²a, and R^{2b} of formula (IV) are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, substituted or unsubstituted 5 to 6 membered heteroaryl, —NR^{a}R^{b}, —CH₂NR^{a}R^{b}, —C(O)R^{a}, —CO₂R^{a}, and —CONR^{a}R^{b}; wherein, optionally, R² and one of R²a or R^{2b} are combined together to form a substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₆ alkyl; and wherein the substituted C₁₋₆ alkyl, substituted phenyl, substituted C₃₋₇ cycloalkyl, and substituted 3 to 7 membered heterocycloalkyl can be substituted with C₁₋₃ alkyl, -halogen, —OH, —OCH₃, —NH₂, —N(CH₃)₂, —CF₃, —CO₂H, —CH₂CO₂H, —CH₂CONH₂, —COOCH₃, —COCH₃, —CONH₂, —CON(CH₃)₂, —OCF₃, —CN, —NO₂, or phenyl.

In some embodiments, the 1,2-diol moiety of the 1,2-diol modified multi-arm PEG polymer backbone comprises a 1,2-cis-diol group of formula (IVA):

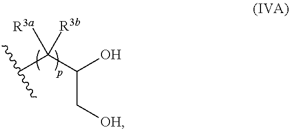

wherein R^{3a} and R^{3b} are each independently selected from the group consisting of hydrogen, substituted or unsubstituted C₁₋₃ alkyl, methoxy, ethoxy, fluoro, chloro, bromo, iodo, —OH, —NR^{a}R^{b}, —CH₂NR^{a}R^{b}, —C(O)R^{a}, and —C(O)OR^{a}; wherein, optionally, one or more of R^{3a} and R^{3b} are combined together to form ═O, substituted or unsubstituted C₃₋₇ cycloalkyl, or substituted or unsubstituted 3 to 7 membered heterocycloalkyl; R^{a} and R^{b} are each independently selected from the group consisting of hydrogen and C₁₋₃ alkyl; and subscript p is an integer from 1-6. In some embodiments, R^{3a} and R^{3b} of formula (IVA) are each independently selected from the group consisting of hydrogen, substituted or unsubstituted C₁₋₃ alkyl, methoxy, ethoxy, fluoro, chloro, bromo, iodo, —OH, —NH₂, —N(CH₃)₂, —CO₂H, —COOCH₃, —COCH₃, and —OCF₃, wherein the substituted C₁₋₃ alkyl, substituted C₃₋₇ cycloalkyl, and substituted 3 to 7 membered heterocycloalkyl can be substituted with —OH, —OCH₃, —NH₂, —N(CH₃)₂, —CF₃, —CO₂H, —CH₂CO₂H, —CH₂CONH₂, —COOCH₃, —COCH₃, —CONH₂, —CON(CH₃)₂, —CN, or —NO₂; and subscript p is an integer from 1-6. In some embodiments, subscript p of formula (IVA) is an integer selected from 2, 3, 4, 5, or 6. In some embodiments, subscript p of formula (IVA) is an integer selected from 2-6. In some embodiments, subscript p of formula (IVA) is an integer selected from 3-6. In some embodiments, subscript p of formula (IVA) is an integer selected from 4-6. In some embodiments, subscript p is 2. In some embodiments, subscript p is 3. In some embodiments, subscript p is 4. In some embodiments, subscript p is 5. In some embodiments, subscript p is 6.

In some embodiments, the 1,2-diol moiety of the 1,2-diol modified multi-arm PEG polymer backbone comprises a 1,2-cis-diol group of formula (IV) or formula (IVA) selected from the group consisting of:

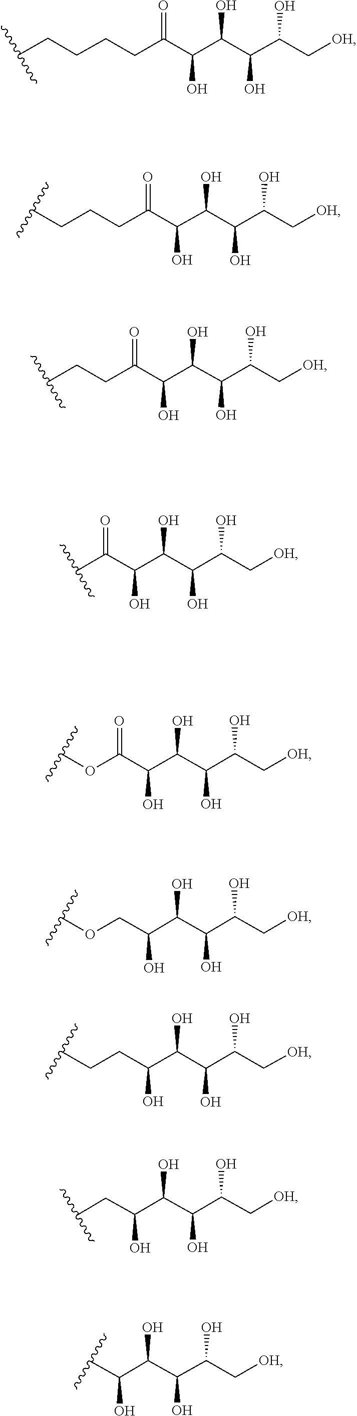

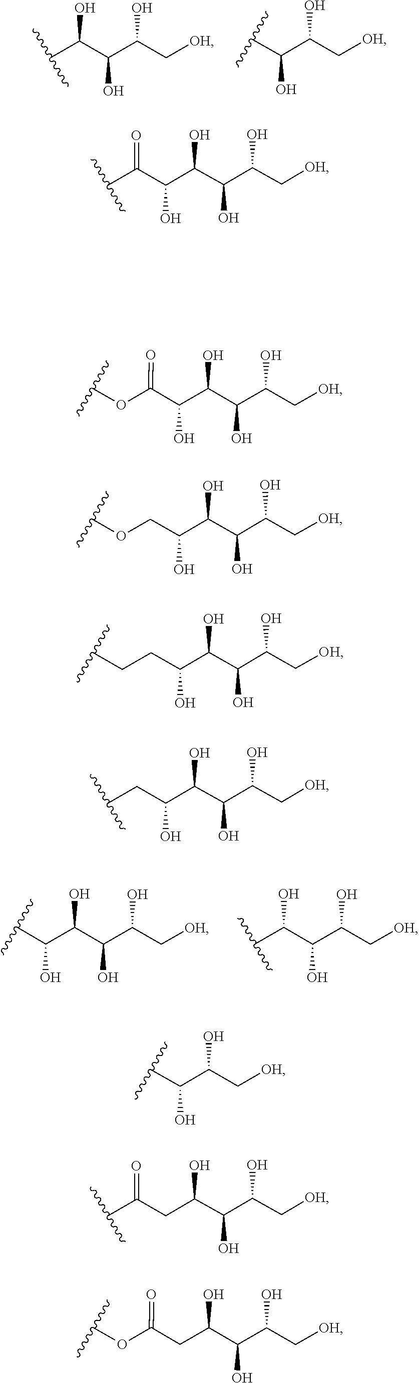

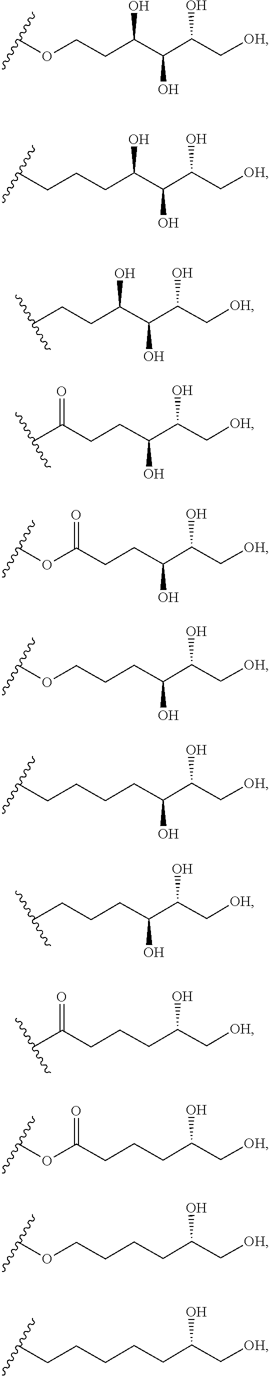

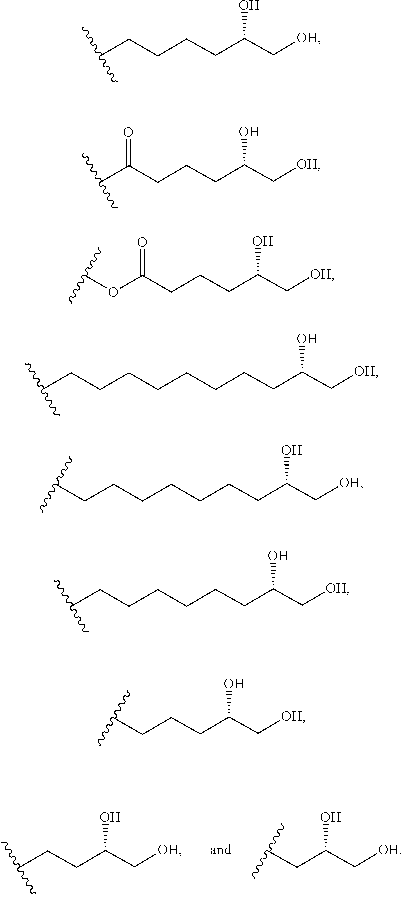

In some embodiments, the 1,2-diol moiety of the 1,2-diol modified multi-arm PEG polymer backbone comprises a 1,2-cis-diol group of formula (IV) or formula (IVA) selected from the group consisting of:

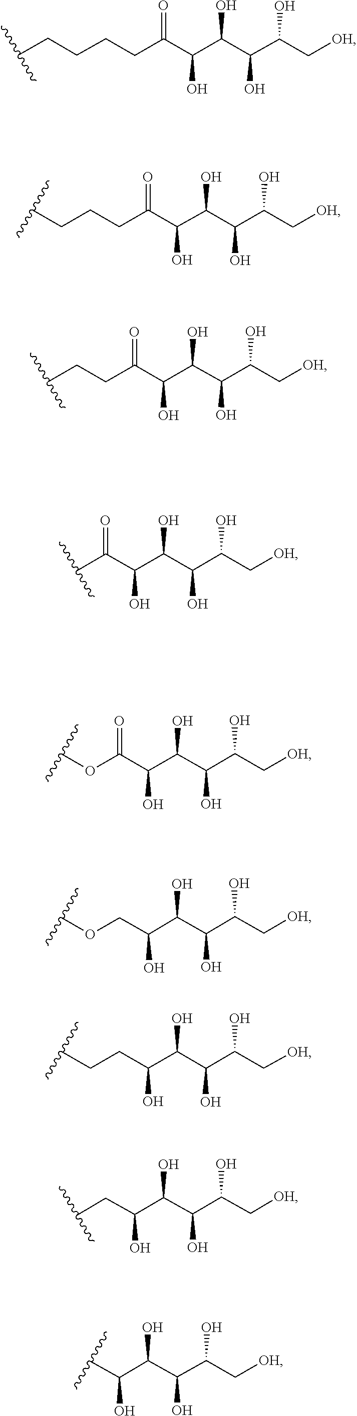

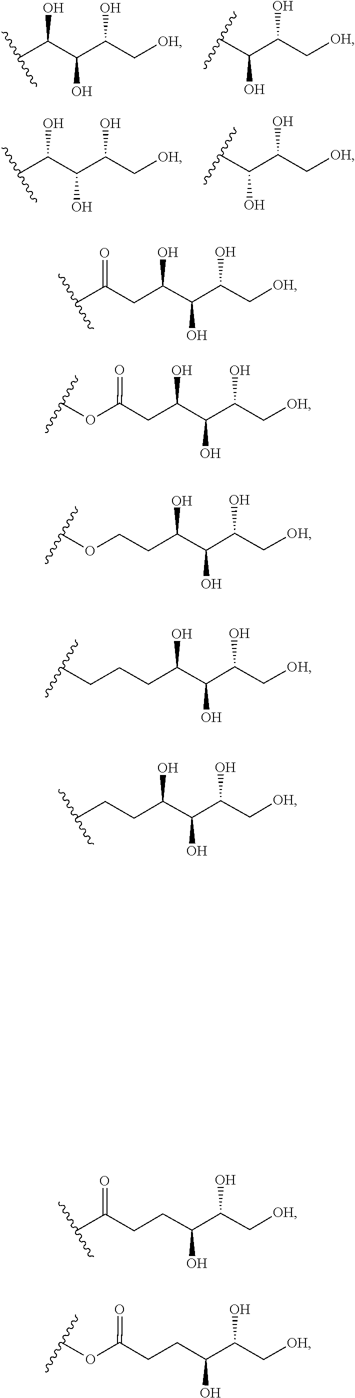

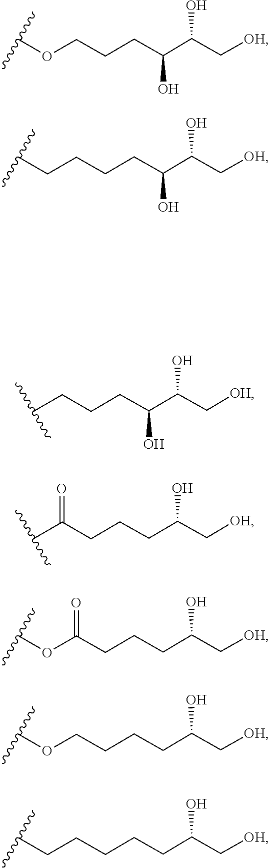

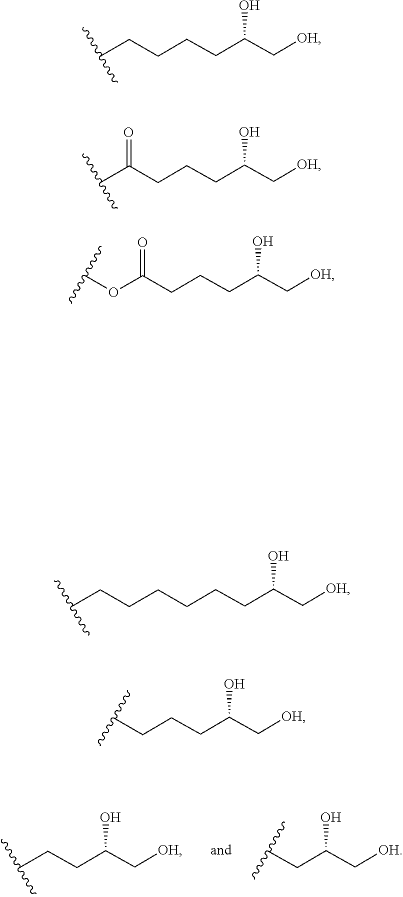

In some embodiments, the 1,2-diol moiety of the 1,2-diol modified multi-arm PEG polymer backbone comprises a 1,2-cis-diol group of formula (IVA) selected from the group consisting of:

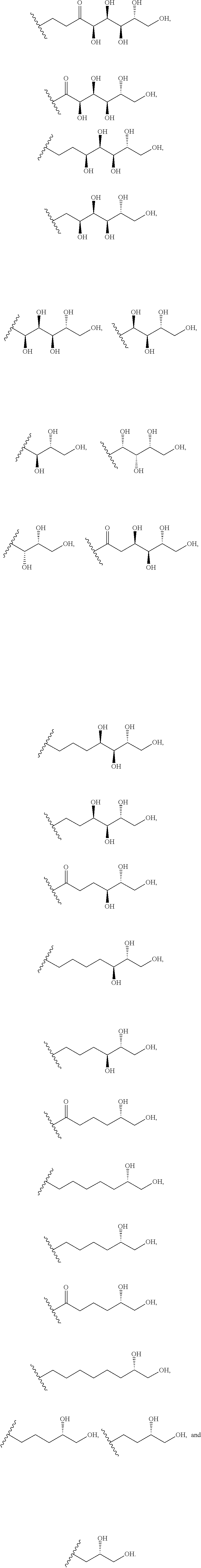

In some embodiments, the 1,2-diol moiety of the 1,2-diol modified multi-arm PEG polymer backbone comprises a 1,2-cis-diol group of formula (IVA) selected from the group consisting of:

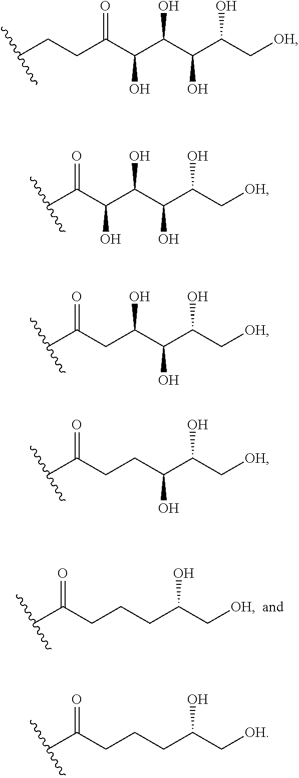 [SMILES: *CCC(=O)[C@H](O)[C@@H](O)[C@H](O)[C@H](O)CO ; *C(=O)[C@H](O)[C@@H](O)[C@H](O)[C@H](O)CO ; *C(=O)C[C@@H](O)[C@H](O)[C@H](O)CO ; *C(=O)CC[C@H](O)[C@H](O)CO ; *C(=O)CCC[C@H](O)CO ; *C(=O)CCC[C@H](O)CO]

### Therapeutic Agents

The dynamic polymeric hydrogel networks described herein are used to stabilize one or more therapeutic agents. Thus, the dynamic polymeric hydrogel compositions of the present invention can be subjected to environmental stressors for a period of time while retaining at least 50% of the therapeutic agents' original bioactivity within the hydrogel network of cross-linked PBA-PEG and 1,2-diol-PEG backbones. Any suitable therapeutic agent is useful in the dynamic polymeric hydrogel compositions of the present invention. In general, a stabilized therapeutic agent encapsulated within a dynamic polymeric hydrogel is resistant to thermal and chemical aggregation, degradation or fragmentation under given manufacture, preparation, transportation and storage conditions.

In some embodiments, the one or more therapeutic agent of the dynamic polymeric hydrogel composition is selected from the group consisting of a protein, peptide, antigen, immunogen, enzyme, cell therapy, antibiotic, anesthetic, antibody or portions thereof (e.g., antibody-like molecules), nucleic acid (e.g., oligonucleotides, polynucleotides, siRNA, shRNA), aptamer, growth factor, bacteria, diagnostic agent such as a contrast agent or dye, mammalian cells (e.g., human embryonic cells), hormone, anti-inflammatory agent, analgesic, cardiac agent, vaccine, virus, viral vector, psychotropic agent, and combinations thereof.

Examples of proteins, such as enzymes and antibodies, include without limitation, cell signaling proteins (TNFα), Lysozyme, Alkaline phosphatase (ALP), Adenosine deaminase, L-Asparaginase, β-galactosidase, isomerases (such as, topoisomerase I-IV, DNA gyrase), Mammalian urate oxidase, Interferons, Anti-TNF α Fab, granulocyte colony stimulated factor (G-CSF), Continuous erythropoietin receptor activator, hGH antagonist B2036, Insulin, Insulin human inhalation, Insulin aspart, Insulin glulisine, Insulin lispro, Isophane insulin, Insulin detemir, Insulin glargine, Insulin zinc extended, Pramlintide acetate, Growth hormone (GH), Somatotropin, Mecasermin, Mecasermin rinfabate, Factor VIII. Factor IX, Antithrombin III (AT-iii), fibroblast growth factor (FGF), basic fibroblast growth factor (bFGF), vascular endothelial growth factor (VEGF), platelet derived growth factor (PDGF), Protein C concentrate, β-Gluco-cerebrosidase, Alglucosidase-α, Laronidase (α-L-iduronidase), Idursulphase (iduronate-2-sulphatase), Galsulphase, Agalsidase-0 (human α-galactosidase A), α-1-Proteinase inhibitor, Lactase, Pancreatic enzymes, lipase, amylase, protease, Adenosine deaminase, Pooled immunoglobulins, Human albumin, Erythropoietin, Epoetin-α, Darbepoetin-α, Sargramostim (granulocytemacrophage colony stimulating factor; GM-CSF), Oprelvekin (interleukin 11; IL11) Human follicle-stimulating hormone (FSH), Human chorionic gonadotropin (HCG), Lutropin-α, Type I alpha-interferon, interferon alfacon 1, consensus interferon, Aldesleukin (interleukin 2 (IL2), epidermal thymocyte activating factor (ETAF), Alteolase (tissue plasminogen activator: tPA), Reteplase (deletion mutein of tPA), Tenecteplase, Urokinase, Factor VIIa, Drotrecogin-α (activated protein C), Salmon calcitonin, Teriparatide (human parathyroid hormone residues 1-34), Exenatide, Octreotide, Dibotermin-α (recombinant human bone morphogenic protein 2; rhBMP2), Recombinant human bone morphogenic protein 7 (rhBMP7), Histrelin acetate (gonadotropin releasing hormone; GnrH), Palifermin (keratinocyte growth factor; KGF), Becaplermin (platelet-derived growth factor, PDGF), Trypsin, Nesiritide, Botulinum toxin type A, Botulinum toxin type B, Collages, Collagenase, Human deoxyribonuclease I, dornase-α, Hyaluronidase (bovine, ovine), Hyaluronidase (recombinant human), Papain, L-Asparaginase, Rasburicase, Lepirudin, Bivalirudin, Streptokinase, Anistreplase (anisoylated plasminogen streptokinase activator complex; APSAC), Bevacizumab, Cetuximab, Panitumumab, Alemtuzumab, Rituximab, Trastuzumab, Abatacept Anakinra, HUMIRA® (adalimumab), anti-CD3 monoclonal antibody (such as, muromonab-CD3), STELARA® (ustekinumab), Etanercept, Infliximab, Alefacept, Efalizumab, Natalizumab, Eculizumab, Antithymocyte globulin (rabbit), Basiliximab, Daclizumab, Omalizumab, Palivizumab, Enfuvirtide, Abciximab, Crotalidae polyvalent immune Fab (ovine), Digoxin immune serum Fab (ovine), Ranibizumab, Denileukin diftitox, Ibritumomab tiuxetan, Gemtuzumab ozogamicin, Tositumomab, and itositumomab.

Examples of growth factors include, without limitation, gonadotropin releasing hormone transforming growth factor (TGF); fibroblast growth factor (FGF); nerve growth factor (NGF), growth hormone releasing factor (GHRF), epidermal growth factor (EGF), connective tissue activated osteogenic factors, fibroblast growth factor homologous factor (FGFHF), insulin growth factor (IGF), stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoeitins (Ang), bFGF, HNF, NGF, bone morphogenic protein (BMP), fibroblast growth factor (FGF), hepatocyte growth factor, interleukin (IL)-3, IL-1α, IL-1β, IL-6, IL-7, IL-8, IL-11, and IL-13, colony-stimulating factors, thrombopoietin, erythropoietin, fit3-ligand, and tumor necrosis factor α (TNFα).

Examples of mammalian cells include, without limitation, human umbilical vein endothelial cells (HUVEC), Chinese hamster ovary (CHO) cells, HeLa cells, Madin-Darby canine kidney (MDCK) cells, baby hamster kidney (BHK cells), NSO cells, MCF-7 cells, MDA-MB-438 cells, U87 cells, A172 cells, HL60 cells, A549 cells, SP10 cells, DOX cells, DG44 cells, human embryonic cells (HEK293 cells), SHSY5Y, Jurkat cells, BCP-1 cells, COS cells, Vero cells, GH3 cells, 9L cells, 3T3 cells, MC3T3 cells, C3H-10T½ cells, NIH-3T3 cells, and C6/36 cells.

Examples of antibiotics include, without limitation, penicillins, cephalosporins, penems, carbapenems, monobactams, aminoglycosides, sulfonamides, macrolides, tetracyclins, lincosides, quinolones, chloramphenicol, vancomycin, metronidazole, rifampin, isoniazid, spectinomycin, trimethoprim, and sulfamethoxazole. Examples of anesthetics include, without limitation, general anesthetics, such as inhalation anesthetics, halogenated inhalation anesthetics, intravenous anesthetics, barbiturate intravenous anesthetics, benzodiazepine intravenous anesthetics, and opiate agonist intravenous anesthetics. Examples of hormones include, without limitation, hormone modifiers, abortifacients, adrenal agents, corticosteroid adrenal agents, androgens, anti-androgens, antidiabetic agents, sulfonylurea antidiabetic agents, antihypoglycemic agents, oral contraceptives, progestin contraceptives, estrogens, fertility agents, oxytocics, parathyroid agents, pituitary hormones, estrogens, progestins, antithyroid agents, thyroid hormones, and tocolytics.

Examples of analgesics include, without limitation, acetaminophen, salicylates (such as aspirin, ASA, enteric coated ASA), lidocaine, diclofenac, ibuprofen, ketoprofen, naproxen, codeine, fentanyl, hydromorphone, and morphine. Alternatively, the therapeutic agent can be an anti-inflammatory agent, such as a steroid. Examples of cardiac agents include, without limitation, nitrates, β-blockers, calcium channel blockers, diuretic agents, vasodilator agents, positive inotropes, ACE inhibitors and aldosterone antagonists (e.g. spironolactone), blood thinning therapeutics (e.g., aspirin, heparins, warfarins) and nitroglycerin. Examples of psychotropic agents include, without limitation, antidepressants, heterocyclic antidepressants, monoamine oxidase inhibitors, selective serotonin re-uptake inhibitors, tricyclic antidepressants, antimanics, antipsychotics, phenothiazine antipsychotics, anxiolytics, sedatives, and hypnotics.

Examples of viruses include, without limitation, dsDNA viruses (e.g. Adenoviruses and Adeno-associated viruses, Herpesviruses, Poxviruses), ssDNA viruses (e.g. Parvoviruses), dsRNA viruses (e.g. Reoviruses), (+)ssRNA viruses (e.g. Picornaviruses, Togaviruses), (−)ssRNA viruses (e.g. Orthomyxoviruses, Rhabdoviruses), ssRNA-RT viruses, i.e., (+)sense RNA with DNA intermediate in life-cycle (e.g. Retroviruses), and dsDNA-RT viruses (e.g. Hepadnaviruses). Examples of vaccines include, without limitation, vaccine products, influenza vaccine, cholera vaccine, bubonic plague vaccine, polio vaccine, hepatitis A vaccine, and rabies vaccine.

In some embodiments, the vaccine can be a vaccine product, such as, for example, BIOTHRAX® (anthrax vaccine adsorbed, Emergent Biosolutions, Rockville, Md.); TICE® BCG Live (Bacillus Calmette-Guerin for intravesical use, Organon Tekina Corp. LLC, Durham, N.C.); MYCOBAX BCG Live (Sanofi Pasteur Inc); DAPTACEL® (diphtheria and tetanus toxoids and acellular pertussis [DTaP] vaccine adsorbed, Sanofi Pasteur Inc.); INFANRIX® (DTaP vaccine adsorbed, GlaxoSmithKline); TRIPEDIA® (DTaP vaccine, Sanofi Pasteur); TRIHIBIT® (DTaP/Hib#, sanofi pasteur); KINRIX® (diphtheria and tetanus toxoids, acellular pertussis adsorbed and inactivated poliovirus vaccine, GlaxoSmithKline); PEDIARIX® (DTaP-HepB-IPV, GlaxoSmithKline); PENTACEL® (diphtheria and tetanus toxoids and acellular pertussis adsorbed, inactivated poliovirus and Haemophilus b conjugate [tetanus toxoid conjugate] vaccine, sanofi pasteur); Diphtheria and Tetanus Toxoids, adsorbed (for pediatric use, Sanofi Pasteur); DECAVAC® (diphtheria and tetanus toxoids adsorbed, for adult use, Sanofi Pasteur); ACTHIB® (Haemophilus b tetanus toxoid conjugate vaccine, Sanofi Pasteur); PEDVAXHIB® (Hib vaccine, Merck); Hiberix (Haemophilus b tetanus toxoid conjugate vaccine, booster dose, GlaxoSmithKline); COMVAX® (Hepatitis B-Hib vaccine, Merck); HAVRIX® (Hepatitis A vaccine, pediatric, GlaxoSmithKline); VAQTA® (Hepatitis A vaccine, pediatric, Merck); ENGERIX-B® (Hep B, pediatric, adolescent, GlaxoSmithKline); RECOMBIVAX HBO (hepatitis B vaccine, Merck); TWINRIX® (HepA/HepB vaccine, 18 years and up, GlaxoSmithKline); CERVARIX® (human papillomavirus bivalent [types 16 and 18] vaccine, recombinant, GlaxoSmithKline); GARDASIL® (human papillomavirus bivalent [types 6, 11, 16 and 18] vaccine, recombinant, Merck); AFLURIA® (Influenza vaccine, 18 years and up, CSL); AGRIFLU™ (influenza virus vaccine for intramuscular injection, Novartis Vaccines); FLUARIX® (Influenza vaccine, 18 years and up, GaxoSmithKline); FLULAVAL® (Influenza vaccine, 18 years and up, GaxoSmithKline); FLUVIRIN® (Influenza vaccine, 4 years and up, Novartis Vaccine); FLUZONE® (Influenza vaccine, 6 months and up, Sanofi Pasteur); FLUMIST® (Influenza vaccine, 2 years and up, MedImmune); IPOL® (e-IPV polio vaccine, sanofi Pasteur); JE-VAX® (Japanese encephalitis virus vaccine inactivated, BIKEN, Japan); IXIARO® (Japanese encephalitis virus vaccine inactivated, Novarits); MENACTRA® (Meningococcal [Groups A, C, Y and W-135] and diphtheria vaccine, Sanofi Pasteur); MENOMUNE®-A/C/Y/W-135 (Meningococcal polysaccharide vaccine, sanofi pasteur); MMRII® (MMR vaccine, Merck); MENVEO® (Meningococcal [Groups A, C, Y and W-135] oligosaccharide diphtheria CRM197 conjugate vaccine, Novartis Vaccines); PROQUAD® (MMR and varicella vaccine, Merck); PNEUMOVAX 23® (pneumococcal polysaccharide vaccine, Merck); PREVNAR® (pneumococcal vaccine, 7-valent, Wyeth/Lederle); PREVNAR-13@(pneumococcal vaccine, 13-valent, Wyeth/Lederle); POLIOVAX™ (poliovirus inactivated, sanofi pasteur); IMOVAX® (Rabies vaccine, Sanofi Pasteur); RABAVERT™ (Rabies vaccine, Chiron); ROTATEQ® (Rotavirus vaccine, live, oral pentavalent, Merck); ROTARIX® (Rotavirus, live, oral vaccine, GlaxoSmithKline); DECAVAC™ (tetanus and diphtheria toxoids vaccine, sanofi pasteur); Td (generic) (tetanus and diphtheria toxoids, adsorbed, Massachusetts Biol. Labs); TYPHIMVI@ (typhoid Vi polysaccharide vaccine, Sanofi Pasteur); ADACEL® (tetanus toxoid, reduced diphtheria toxoid and acellular pertussis, sanofi pasteur); BOOSTRIX® (tetanus toxoid, reduced diphtheria toxoid and acellular pertussis, GlaxoSmithKline); VIVOTIF® (typhoid vaccine live oral Ty21a, Berna Biotech); ACAM2000™ (Smallpox (vaccinia) vaccine, live, Acambis, Inc.); DRYVAX® (Smallpox (vaccinia) vaccine); VARIVAX® (varicella [live] vaccine, Merck); YF-VAX® (Yellow fever vaccine, Sanofi Pasteur); ZOSTAVAX® (Varicella zoster, Merck); or combinations thereof.

In some embodiments, the one or more therapeutic agent of the dynamic polymeric hydrogel composition is selected from the group consisting of an enzyme, cell therapy, antibiotic, anesthetic, antibody, growth factor, human embryonic cells, protein, hormone, anti-inflammatory agent, analgesic, cardiac agent, vaccine, and psychotropic agent. Examples of enzymes include, without limitation, kinases, dehydrogenases, oxidoreductases, GTPases, carboxyl transferases, acyl transferases, decarboxylases, transaminases, racemases, methyl transferases, formyl transferases, and α-ketodecarboxylases, Alkaline phosphatase (ALP), β-galactosidase, isomerases (e.g., topoisomerase I-IV, DNA gyrase). In some embodiments, the one or more therapeutic agent of the dynamic polymeric hydrogel composition is selected from the group consisting of β-galactosidase, a vaccine, Topoisomerase I-IV, HEK293 cells, DNA gyrase, HUMIRA® (adalimumab), anti-CD3 monoclonal antibody, STELARA® (ustekinumab), TNFα, influenza vaccine, adenovirus, and adeno-associated virus.

### Encapsulation

The present invention described methods by which to stabilize a therapeutic agent, or multiple therapeutic agents, of a dynamic polymeric hydrogel composition described herein. In some embodiments, the method for stabilizing the one or more therapeutic agent involves encapsulating the therapeutic agent, or multiple therapeutic agents, within the dynamic polymeric hydrogel network. The method of encapsulating the therapeutic agent, or multiple therapeutic agents comprises a series of combination and mixing steps involving the components of the dynamic polymeric hydrogel composition. Combining the PBA modified multi-arm PEG polymer backbone of formula (I), 1,2-diol modified multi-arm PEG polymer backbone of formula (II), and the one or more therapeutic agents, per the methods described herein, results in the formation of the dynamic polymeric hydrogel composition, in which the one or more therapeutic agents is encapsulated within and, thus, stabilized by the dynamic polymeric hydrogel network of PBA-1,2-diol cross-linked PEG polymer backbones.

In some embodiments, the method for stabilizing therapeutic agents comprising encapsulating the therapeutic agents involves (a) admixing the therapeutic agent with a solution of the 1,2-diol modified multi-arm PEG polymer backbone of formula (II) to form a therapeutic agent diol-PEG admixture; and (b) adding a solution of the PBA modified multi-arm PEG polymer backbone of formula (I) to the therapeutic agent diol-PEG admixture to form the dynamic polymeric hydrogel composition with the therapeutic agent encapsulated therein, thereby stabilizing the encapsulated therapeutic agent. In some embodiments, encapsulation of the therapeutic agents comprises (a) admixing the therapeutic agent with a solution of the PBA modified multi-arm PEG polymer backbone of formula (I) to form a therapeutic agent PBA-PEG admixture; and (b) adding a solution of the 1,2-diol modified multi-arm PEG polymer backbone of formula (II) to the therapeutic agent PBA-PEG admixture to form the dynamic polymeric hydrogel composition with the therapeutic agent encapsulated therein, thereby stabilizing the encapsulated therapeutic agent.

The admixing step of the encapsulation and stabilization methods described herein involve the combination or mixing of the one or more therapeutic agents with either the PBA modified PEG polymer backbone component of the pre-gelled dynamic hydrogel composition or the 1,2-diol modified PEG polymer backbone component of the pre-gelled dynamic hydrogel composition. The admixing is performed in a manner such that the components within the population are distributed and evenly dispersed throughout the pre-gelled hydrogel admixture. In other words, the one or more therapeutic agents is mixed thoroughly with either the PBA-PEG component or 1,2-diol-PEG component of the pre-gelled dynamic hydrogel composition to form a homogenous solution of either a therapeutic agent diol-PEG admixture or a therapeutic agent PBA-PEG admixture. In some embodiments, admixing the components of the pre-gelled hydrogel admixture involves vortexing, sonicating, or homogenizing, and, optionally, heating to completely dissolve and disperse the one or more therapeutic agents within a solution of either the PBA-PEG polymer backbone of formula (I) or the 1,2-diol-PEG polymer backbone of formula (II), thereby forming either the therapeutic agent diol-PEG admixture or a therapeutic agent PBA-PEG admixture.

In some embodiments, admixing the components of the pre-gelled hydrogel admixture involves vortexing, sonicating, or homogenizing, and, optionally, heating the one or more therapeutic agents with a solution of either the PBA-PEG component or 1,2-diol-PEG component for a duration of from 1 to 60 minutes, or longer. In some embodiments, admixing the components of the pre-gelled hydrogel admixture involves vortexing, sonicating, or homogenizing, and, optionally, heating the one or more therapeutic agents with a solution of either the PBA-PEG component or 1,2-diol-PEG component for a duration of 1 minute, 2 minutes, 3, 4, 5, 10, 15, 20, 25, 30, 35, 40, 45, 50, 55, or 60 minutes. In some embodiments, admixing the components of the pre-gelled hydrogel admixture involves vortexing, sonicating, or homogenizing, and, optionally, heating the one or more therapeutic agents with a solution of either the PBA-PEG component or 1,2-diol-PEG component for a duration of from 1 to 50 minutes, 2 to 45, 3 to 40, 4 to 35, 5 to 30, 10 to 25, or from 15 to 20 minutes. In some embodiments, admixing the components of the pre-gelled hydrogel admixture involves vortexing, sonicating, or homogenizing, and, optionally, heating the one or more therapeutic agents with a solution of either the PBA-PEG component or 1,2-diol-PEG component for a duration of from 1 to 5 minutes, 1 to 10, 1 to 15, 1 to 20, 1 to 25, or from 1 to 30 minutes, or longer, until the one or more therapeutic agents is dissolved within the solution of either the PBA-PEG component or 1,2-diol-PEG component.

In some embodiments, admixing the components of the pre-gelled hydrogel admixture involves vortexing, sonicating, or homogenizing at a temperature of from about 10° C. to about 110° C. In some embodiments, admixing the components of the pre-gelled hydrogel admixture involves vortexing, sonicating, or homogenizing at a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., or about 110° C. In some embodiments, admixing the components of the pre-gelled hydrogel admixture involves vortexing, sonicating, or homogenizing at a temperature of from about 10° C. to about 100° C., 15° C. to 95° C., 20° C. to 90° C., 25° C. to 85° C., 30° C. to 80° C., 35° C. to 75° C., 40° C. to 70° C., 45° C. to 65° C., or from about 50° C. to about 60° C. In some embodiments, admixing the components of the pre-gelled hydrogel admixture involves vortexing, sonicating, or homogenizing at a temperature of from about 15° C. to about 70° C., 20° C. to 60° C., 25° C. to 50° C., or from about 30° C. to about 40° C. In some embodiments, admixing the components of the pre-gelled hydrogel admixture involves vortexing, sonicating, or homogenizing at room temperature, e.g., between 20° C. and 35° C. The temperature at which admixing the components of the pre-gelled hydrogel admixture involves vortexing, sonicating, or homogenizing occurs will depend upon the temperature sensitivity of the therapeutic agent(s) and the PBA-PEG component or 1,2-diol-PEG component.

Following the admixing step of the encapsulation methods, either a solution of the PBA modified multi-arm PEG polymer backbone of formula (I) is added to the therapeutic agent diol-PEG admixture or a solution of the 1,2-diol modified multi-arm PEG polymer backbone of formula (II) is added to the therapeutic agent PBA-PEG admixture. In some embodiments, a solution of the PBA modified multi-arm PEG polymer backbone of formula (I) is added to the therapeutic agent diol-PEG admixture to form the dynamic polymeric hydrogel composition with the therapeutic agent encapsulated therein, thereby stabilizing the encapsulated therapeutic agent. In some embodiments, a solution of the 1,2-diol modified multi-arm PEG polymer backbone of formula (II) is added to the therapeutic agent PBA-PEG admixture to form the dynamic polymeric hydrogel composition with the therapeutic agent encapsulated therein, thereby stabilizing the encapsulated therapeutic agent.

In some embodiments, formation of the dynamic polymeric hydrogel composition with the therapeutic agent encapsulated therein involves vortexing, sonicating, or homogenizing, and, optionally, heating either the solution of the PBA-PEG component with the therapeutic agent 1,2-diol-PEG admixture or the solution of the 1,2-diol-PEG component with the therapeutic agent PBA-PEG admixture for a duration of from 1 to 300 seconds, or longer, or until the hydrogel is formed. In some embodiments, formation of the dynamic polymeric hydrogel composition with the therapeutic agent encapsulated therein involves vortexing, sonicating, or homogenizing, and, optionally, heating either the PBA-PEG solution or 1,2-diol-PEG solution with the appropriate therapeutic agent admixture for a duration of 1 second, or2 seconds, 3, 4, 5, 10, 15, 20, 25, 30, 40, 50, 60, 70, 80, 90, 100, 125, 150, 175, 200, 225, 250, 275, or 300 seconds, or until the hydrogel is formed. In some embodiments, formation of the dynamic polymeric hydrogel composition with the therapeutic agent encapsulated therein involves vortexing, sonicating, or homogenizing, and, optionally, heating the PBA-PEG solution or 1,2-diol-PEG solution with the appropriate therapeutic agent admixture for a duration of from about 1 to about 275 seconds, or from about 2 to about 250 seconds, 3 to 225, 4 to 200, 5 to 175, 10 to 150, 15 to 125, 20 to 100, 25 to 90, 30 to 80, 40 to 70, or from about 50 to about 60 seconds, or until the hydrogel is formed. In some embodiments, formation of the dynamic polymeric hydrogel composition with the therapeutic agent encapsulated therein involves vortexing, sonicating, or homogenizing, and, optionally, heating the PBA-PEG solution or 1,2-diol-PEG solution with the appropriate therapeutic agent admixture for a duration of from about 1 to about 120 seconds, or from about 1 to about 90 seconds, from about 1 to about 60 seconds, or from about 1 to about 30 seconds, or until the hydrogel is formed.

In some embodiments, formation of the dynamic polymeric hydrogel composition with the therapeutic agent encapsulated therein involves vortexing, sonicating, or homogenizing the PBA-PEG solution or 1,2-diol-PEG solution with the appropriate therapeutic agent admixture at a temperature of from about 10° C. to about 110° C. In some embodiments, formation of the dynamic polymeric hydrogel composition with the therapeutic agent encapsulated therein involves vortexing, sonicating, or homogenizing the PBA-PEG solution or 1,2-diol-PEG solution with the appropriate therapeutic agent admixture at a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., or about 110° C. In some embodiments, formation of the dynamic polymeric hydrogel composition with the therapeutic agent encapsulated therein involves vortexing, sonicating, or homogenizing the PBA-PEG solution or 1,2-diol-PEG solution with the appropriate therapeutic agent admixture at a temperature of from about 10° C. to about 100° C., 15° C. to 95° C., 20° C. to 90° C., 25° C. to 85° C., 30° C. to 80° C., 35° C. to 75° C., 40° C. to 70° C., 45° C. to 65° C., or from about 50° C. to about 60° C.

In some embodiments, formation of the dynamic polymeric hydrogel composition with the therapeutic agent encapsulated therein involves vortexing, sonicating, or homogenizing the PBA-PEG solution or 1,2-diol-PEG solution with the appropriate therapeutic agent admixture at a temperature of from about 15° C. to about 70° C., 20° C. to 60° C., 25° C. to 50° C., or from about 30° C. to about 40° C. In some embodiments, formation of the dynamic polymeric hydrogel composition with the therapeutic agent encapsulated therein involves vortexing, sonicating, or homogenizing the PBA-PEG solution or 1,2-diol-PEG solution with the appropriate therapeutic agent admixture at room temperature, e.g., between 20° C. and 35° C. The temperature at which formation of the dynamic polymeric hydrogel composition with the therapeutic agent encapsulated therein involving vortexing, sonicating, or homogenizing the PBA-PEG solution or 1,2-diol-PEG solution with the appropriate therapeutic agent occurs will depend upon the temperature sensitivity of the therapeutic agent(s) and the PBA-PEG component or 1,2-diol-PEG component.

The PBA-PEG polymer backbone of formula (I), the 1,2-diol-PEG polymer backbone of formula (II), and the one or more therapeutic agents can be combined in any amount and in any ratio sufficient to form the dynamic polymeric hydrogel compositions, within which the one or more therapeutic agents are encapsulated. For example, the stoichiometric ratio (or mole ratio) of the PBA modified multi-arm PEG polymer backbone of formula (I) to the 1,2-diol modified multi-arm PEG polymer backbone of formula (II) can range from 20:1 to 1:20, 18:1 to 1:18, 15:1 to 1:15, 12:1 to 1:12, 10:1 to 1:10, 8:1 to 1:8, 5:1 to 1:5, 3:1 to 1:3, or from 2:1 to 1:2. In some embodiments, the stoichiometric ratio of the PBA-PEG polymer backbone of formula (I) to the 1,2-diol-PEG polymer backbone of formula (II) can be 18:1, 18:5, 18:7, 18:11, 18:13, 18:17, 17:18, 13:18, 11:18, 7:18, 5:18, 1:18, 15:1, 15:2, 15:4, 15:7, 15:8, 15:11, 15:13, 15:14, 14:15, 13:15, 11:15, 8:15, 7:15, 4:15, 2:15, or 1:15. In some embodiments, the stoichiometric ratio of the PBA-PEG polymer backbone of formula (I) to the 1,2-diol-PEG polymer backbone of formula (II) can be 12:1, 12:5, 12:7, 12:11, 11:12, 7:12, 5:12, 1:12, 10:1, 10:3, 10:7, 10:9, 9:10, 7:10, 3:10, 1:10, 9:1, 9:2, 9:4, 9:5, 9:7, 9:8, 8:9, 7:9, 5:9, 4:9, 2:9, 1:9, 8:1, 8:3, 8:5, 8:7, 7:8, 5:8, 3:8, 1:8, 7:1, 7:2, 7:3, 7:4, 7:5, 7:6, 6:7, 5:7, 4:7, 3:7, 2:7, 1:7, 6:1, 6:5, 5:6, or 6:1.

In some embodiments, the stoichiometric ratio of the PBA modified multi-arm PEG polymer backbone of formula (I) to the 1,2-diol modified multi-arm PEG polymer backbone of formula (II) ranges from 1:5 to 5:1. In some embodiments, the stoichiometric ratio of the PBA-PEG polymer backbone of formula (I) to the 1,2-diol-PEG polymer backbone of formula (II) can be 5:1, 5:2, 5:3, 5:4, 4:5, 3:5, 2:5, 1:5, 4:1, 4:3, 3:4, 1:4, 3:1, 3:2, 2:3, 1:3, 2:1, 1:2, or 1:1. In some embodiments, the stoichiometric ratio of the PBA-PEG polymer backbone of formula (I) to the 1,2-diol-PEG polymer backbone of formula (II) can be 2.1:1.1, 2.1:1.2, 2.1:1.3, 2.1:1.5, 2.1:1.8, 2.1:1.9, 2.1:2.0, 2.0:2.1, 1.9:2.1, 1.8:2.1, 1.5:2.1, 1.3:2.1, 1.2:2.1, or 1.1:2.1. In some embodiments, the stoichiometric ratio of the PBA-PEG polymer backbone of formula (I) to the 1,2-diol-PEG polymer backbone of formula (II) can be 1:1.5, 1.5:1, or 1:1.

The amount of the one or more therapeutic agents used in the stabilization and encapsulation methods and dynamic polymeric hydrogel compositions described herein is dependent upon many factors, such as, for example, the type of therapeutic agent being encapsulated and stabilized within the dynamic polymeric hydrogel compositions, the specific dynamic polymeric hydrogel composition (i.e., specific polymer backbones, PBA derivatives, 1,2-diol moieties, and pharmaceutically acceptable excipients), the external conditions and/or environmental stressors to which the dynamic polymeric hydrogel compositions containing encapsulated therapeutic agents will be exposed, duration of exposure, etc. In some embodiments, the amount of the one or more therapeutic agents used in the stabilization and encapsulation methods and dynamic polymeric hydrogel compositions described herein is present in a therapeutically effective amount. In some embodiments, for ease of administration and dosage uniformity, the therapeutically effective amount of the one or more therapeutic agents encapsulated and stabilized within the dynamic polymeric hydrogel compositions is one unit dose or multiple unit doses. For example, the total therapeutically effective amount of the one or more therapeutic agents encapsulated and stabilized within the dynamic polymeric hydrogel compositions can be divided into multiple unit doses and administered in portions over a period of time suitable to treat to the disease or condition. The therapeutically effective amount and/or dosage unit of the one or more therapeutic agents used in the stabilization and encapsulation methods and dynamic polymeric hydrogel compositions will depend upon factors previously mentioned, as well as, for example, the condition or disease being treated/prevented and the condition or disease severity, the dosing regimen, the age, body weight, general health, sex and diet of the patient being treated, and additional factors well known in the medical arts.

In some embodiments, the amount of the one or more therapeutic agents used in the stabilization and encapsulation methods and dynamic polymeric hydrogel compositions described herein is present in an amount of from about 0.10 mg/mL to about 1000 mg/mL. In some embodiments, the amount of the one or more therapeutic agents of the dynamic polymeric hydrogel compositions is present in an amount of about 0.10 mg/mL, 0.15, 0.20, 0.25, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 7.0, 8.0, 10, 15, 20, 25, 30, 40, 50, 60, 70, 80, 90, 100, 200, 300, 400, 500, 600, 700, 800, 900, or about 1000 mg/mL. In some embodiments, the amount of the one or more therapeutic agents of the dynamic polymeric hydrogel compositions is present in an amount of from about 0.10 mg/mL to about 925 mg/mL, about 0.25 mg/mL to about 850 mg/mL, about 0.50 mg/mL to about 725 mg/mL, about 0.75 mg/mL to about 650 mg/mL, about 1.0 mg/mL to about 525 mg/mL, about 2.5 mg/mL to about 450 mg/mL, about 3.0 mg/mL to about 325 mg/mL, about 4.0 mg/mL to about 250 mg/mL, about 5.0 mg/mL to about 200 mg/mL, about 5.5 mg/mL to about 175 mg/mL, about 6.0 mg/mL to about 150 mg/mL, about 6.5 mg/mL to about 125 mg/mL, or from about 7.5 mg/mL to about 100 mg/mL. In some embodiments, the amount of the one or more therapeutic agents used in the stabilization and encapsulation methods and dynamic polymeric hydrogel compositions described herein is present in an amount of from about 0.10 mg/mL to about 100 mg/mL. In some embodiments, the amount of the one or more therapeutic agents of the dynamic polymeric hydrogel compositions is present in an amount of about 0.10 mg/mL, 0.25 mg/mL, 0.50 mg/mL, 1.0 mg/mL, 2.5 mg/mL, 5.0 mg/mL, 10 mg/mL, 25 mg/mL, 50 mg/mL, 75 mg/mL, or about 100 mg/mL.

In some embodiments, the amount of the one or more therapeutic agents encapsulated and stabilized within the dynamic polymeric hydrogel compositions is a therapeutically effective amount. In some embodiments, the therapeutically effective amount of the one or more therapeutic agents is at least one unit dose. In some embodiments, the therapeutically effective amount of the one or more therapeutic agents is more than one unit dose. In some embodiments, the amount of the one or more therapeutic agents used in the stabilization and encapsulation methods and dynamic polymeric hydrogel compositions described herein is a dosage unit of from about 0.001 mg to about 1000 mg per kilogram of a patient's body weight (i.e., about 0.001-1000 mg/kg). In some embodiments, the amount of the one or more therapeutic agents encapsulated and stabilized within the dynamic polymeric hydrogel compositions is a unit dose of about 0.001, 0.005, 0.01, 0.15, 0.20, 0.25, 0.30, 0.40, 0.50, 0.60, 0.70, 0.80, 0.90, 1.0, 1.5, 2.0, 2.5, 3.0, 3.5, 4.0, 4.5, 5.0, 5.5, 6.0, 7.0, 8.0, 10, 15, 20, 25, 30, 35, 40, 45, 50, 55, 60, 65, 70, 75, 85, 90, 95, 100, 150, 200, 250, 300, 350, 400, 450, 500, 550, 600, 650, 700, 750, 800, 850, 900, 950 or 1000 mg/kg. In some embodiments, the amount of the one or more therapeutic agents of the dynamic polymeric hydrogel compositions is a unit dose of from about 0.005 mg/kg to about 950 mg/kg, about 0.01 mg/kg to about 800 mg/kg, about 0.50 mg/kg to about 650 mg/kg, about 1 mg/kg to about 500 mg/kg, about 2.5 mg/kg to about 375 mg/kg, about 5 mg/kg to about 250 mg/kg, about 7.5 mg/kg to about 200 mg/kg, or from about 10 mg/kg to about 150 mg/kg. In some embodiments, the amount of the one or more therapeutic agents of the dynamic polymeric hydrogel compositions is a unit dose of from about 0.10 mg/kg to about 500 mg/kg.

In general, the amount of dynamic polymeric hydrogel (i.e, the network of PBA-1,2-diol cross-linked PEG polymer backbones) in the composition of the present invention is an amount that allows for encapsulation and stabilization of the one or more therapeutic agents via hydrogel formation. In some embodiments, the amount of PBA-1,2-diol cross-linked PEG polymer backbones in the composition of the present invention ranges from between about 0.5% w/v and about 95% w/v of the entire composition. In some embodiments, the amount of PBA-1,2-diol cross-linked PEG polymer backbones is about 1% w/v of the entire composition, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 40% w/v, about 50% w/v, about 60% w/v, about 70% w/v, or about 80% w/v or more of the entire composition. In some embodiments, the amount of PBA-1,2-diol cross-linked PEG polymer backbones in the composition ranges between about 1% w/v and about 90% w/v of the entire composition, between about 2% w/v and about 80% w/v, between about 4% w/v and about 70% w/v, between about 5% w/v and about 60% w/v, between about 5% w/v and about 50% w/v, between about 6% w/v and about 40% w/v, between about 7% w/v and about 30% w/v, or between about 8% w/w and about 20% w/v of the entire composition. In some embodiments, the amount of PBA-1,2-diol cross-linked PEG polymer backbones in the composition ranges between about 1% w/v and about 50% w/v of the entire composition, between about 2% w/v and about 30% w/v, between about 3% w/v and about 20% w/v, between about 4% w/v and about 15% w/v, between about 4.5% w/v and about 12% w/v, between about 5% w/v and about 10% w/v, between about 5.5% w/v and about 8.5% w/v, or between about 6% w/v and about 8% w/v of the entire composition.

Any ratio of dynamic polymeric hydrogel (i.e, the network of PBA-1,2-diol cross-linked PEG polymer backbones) to therapeutic agent, or multiple therapeutic agents, can be used in the dynamic polymeric hydrogel compositions. In some embodiments, the ratio of polymeric hydrogel to therapeutic agent can range from about 10,000:1 to about 1:1. In some embodiments, the ratio of polymeric hydrogel to therapeutic agent can be about 10,000:1, 5,000:1, 2,500:1, 1,000:1, 750:1, 500:1, 250:1, 200:1, 175:1, 150:1, 100:1, 75:1, 50:1, 25:1, 15:1, 10:1, 5:1, 3:1, or about 1:1. In some embodiments, the ratio of polymeric hydrogel to therapeutic agent can range from about 7,500:1 to about 1:1, about 5,000:1 to about 2:1, about 2,500:1 to about 3:1, about 1,500:1 to about 5:1, about 1,000:1 to about 10:1, about 750:1 to about 15:1, about 500:1 to about 25:1, about 250:1 to about 50:1, about 200:1 to about 75:1, or from about 175:1 to about 100:1. The ratio of the dynamic polymeric hydrogel (i.e, the network of PBA-1,2-diol cross-linked PEG polymer backbones) to therapeutic agent, or multiple therapeutic agents, can vary with a number of factors, including the selection and concentration of the therapeutic agent, the environmental stressors (e.g., storage conditions) and duration, the concentration of the dynamic polymeric hydrogel, and the form of the dynamic polymeric hydrogel composition (e.g., hydrogel solution or lyophilized powder). One of skill in the art can determine appropriate ratios and amounts of the components of the dynamic polymeric hydrogel composition, such as, for example, by measuring the bioactivity of the therapeutic agent retained at various ratios described herein over a pre-defined amount of time under a defined condition (e.g., at a temperature of above 0° C.).

The methods of encapsulating and stabilizing the therapeutic agent, or multiple therapeutic agents, via the formation of the dynamic polymeric hydrogel compositions described herein, are performed at a pH suitable for the formation of the dynamic polymeric hydrogel network. The dynamic polymeric hydrogel network forms upon the cross-linking between the PBA derivatives and the 1,2-diol moieties of the modified PEG polymer backbones via covalent interactions. More specifically, the dynamic polymeric hydrogel network of cross-linked covalent bonds between the PBA derivatives and 1,2-diol moieties is formed at a pH that is greater than the pKa of the phenylboronic acid group of the PBA modified multi-arm PEG polymer backbone. As shown below in Scheme 1, aqueous solutions of the neutral trivalent phenylboronic acid species 1a is in a state of equilibrium with its conjugate base 1b, an anionic tetrahedral boronate species. Without wishing to be bound by theory, complexation of hydroxyboronate anion 1b with 1,2-diol 2b at higher pH is thermodynamically favored compared to complexation of neutral boronic acid 1a with 1,2-diol 2a at lower pH, possibly due to a release of angle strain upon rehybridization of boron from sp² to sp³ (i.e., 1200 vs 109° bond angles). See, Hall, D. G., 2011, Boronic Acids: Preparation and Applications in Organic Synthesis, Medicine and Materials, Second Edition, Wiley-VCH Verlag GmbH & Co. In other words, formation of the tetracoordinate hydroxyboronate ester anion 3b is favored at pH≥PBA-pKa, whereas formation of the neutral tricoordinate boronic ester complex 3a is not favored at pH≤PBA-pKa.

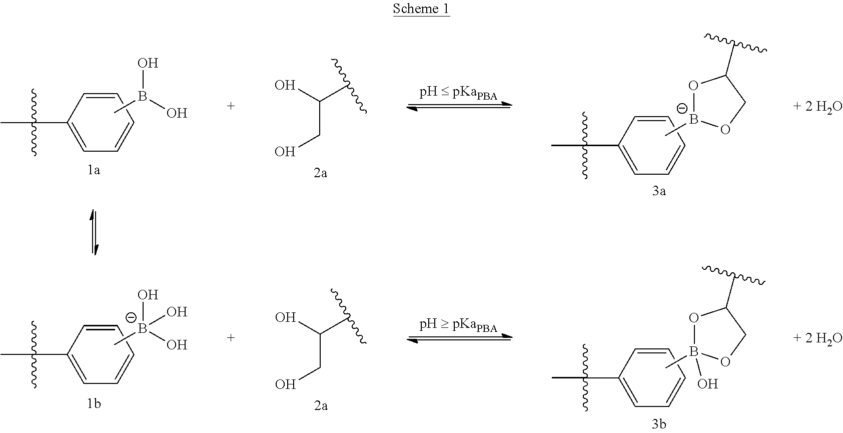

Therefore, the pH at which the dynamic polymeric hydrogel compositions are formed, thereby encapsulating and stabilizing the therapeutic agent, or multiple therapeutic agents, within the dynamic polymeric hydrogel network is dependent upon the pKa of the phenylboronic acid group of the PBA modified multi-arm PEG polymer backbone. The methods of encapsulating and stabilizing the one or more therapeutic agent via the formation of the dynamic polymeric hydrogel compositions described herein are performed at a pH that is greater than the pKa of the phenylboronic acid group of the PBA modified multi-arm PEG polymer backbone. In some embodiments, the pH is about 2.0 or greater. In some embodiments, the pH is about 3.5 or greater, or about 4.0 or greater, about 4.5 or greater, about 5.0 or greater, about 5.5 or greater, about 6.0 or greater, about 6.3 or greater, about 6.5 or greater, about 6.8 or greater, about 7.0 or greater, about 7.1 or greater, about 7.2 or greater, about 7.3 or greater, about 7.4 or greater, about 7.5 or greater, about 7.6 or greater, about 7.7 or greater, or about 7.8 or greater.

In some embodiments, the dynamic polymeric hydrogel composition used for the encapsulation and stabilization of one or more therapeutic agents can contain one or more pharmaceutically acceptable excipients in addition to the hydrogel network of the PBA modified multi-arm PEG polymer backbone components, 1,2-diol modified multi-arm PEG polymer backbone components, and the one or more therapeutic agent. Such optional pharmaceutically acceptable excipient(s) may be incorporated into the dynamic polymeric hydrogel composition prior to the formation of the hydrogel network. In some embodiments, the one or more excipients can be incorporated into the composition during the admixing step of the encapsulation/stabilization methods, wherein the excipient(s) and one or more therapeutic agents are admixed together with the 1,2-diol-PEG or PBA-PEG solution. In some embodiments, the one or more excipients can be incorporated into the composition after the admixing step, and before the adding either the 1,2-diol-PEG or PBA-PEG solution to the admixture. In some embodiments, the one or more excipients can be incorporated into the composition after the admixing step, and simultaneously during the addition of either the 1,2-diol-PEG or PBA-PEG solution to the admixture.

Any suitable pharmaceutically acceptable excipient is useful in the dynamic polymeric hydrogel compositions of the present invention. For example, the dynamic polymeric hydrogel compositions may optionally contain one or more pharmaceutically acceptable carriers, diluents, excipients, or stabilizers typically employed in the art, such as, for example, buffering agents, stabilizing agents, preservatives, isotonifiers, non-ionic detergents, antioxidants, and other miscellaneous additives. See, Remington's Pharmaceutical Sciences, 16th ed., Osol, ed. (1980). Such pharmaceutically acceptable additives are neutral and will not interfere with the effectiveness of the biological activity of the therapeutic agent(s). Moreover, these optional pharmaceutically acceptable excipients will not interfere with cross-linking between the PBA derivatives and the 1,2-diol moieties of the modified PEG polymer backbones.

In some embodiments, the dynamic polymeric hydrogel composition can contain one or more pharmaceutically acceptable excipients such as sterile water, saline, buffered solutions at a suitable pH, Ringer's solution, trehalose solution, Hank's solution, and other aqueous physiologically balanced salt solutions. Non-aqueous pharmaceutically acceptable excipients, such as fixed oils, vegetable oils such as olive oil, peanut oil, soybean oil and sesame oil, mineral oil, triglycerides, propylene glycol, polyethylene glycol, and injectable organic esters such as ethyl oleate can also be used. Other useful additives include suspensions containing viscosity enhancing agents, such as sodium carboxymethylcellulose. Excipients can also be substances that enhance isotonicity and chemical stability.

Buffering agents help to maintain a pH that is suitable for the formation of the dynamic polymeric hydrogel network. Buffers can be present at a concentration ranging from about 2 mM to about 500 M. Suitable buffering agents for use with the dynamic polymeric hydrogel compositions of the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid-monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). In some embodiments, phosphate buffers, carbonate buffers, histidine buffers, trimethylamine salts, such as Tris, HEPES, and other such known buffers can be used

Preservatives may be added to retard microbial growth, and may be added in amounts ranging from 0.2%-1% (w/v). Suitable preservatives for use with the dynamic polymeric hydrogel compositions include phenol, benzyl alcohol, m-cresol, octadecyldimethylbenzyl ammonium chloride, benzyaconium halides (e.g., chloride, bromide and iodide), hexamethonium chloride, alkyl parabens, such as, methyl or propyl paraben, resorcinol, cyclohexanol and 3-pentanol. In some embodiments, additives such as stabilizers can be present in the dynamic polymeric hydrogel compositions, which can range in function from a bulking agent to an additive which solubilizes the therapeutic agent or helps to prevent denaturation or adherence to container walls. Typical stabilizers can be polyhydric sugar alcohols; amino acids, such as arginine, lysine, glycine, glutamine, asparagine, histidine, alanine, ornithine, L-leucine, 2-phenylalanine, glutamic acid, threonine etc.; trehalose; sulfur containing reducing agents, such as urea, glutathione, thioctic acid, sodium thioglycolate, thioglycerol, α-monothioglycerol and sodium thiosulfate; low molecular weight polypeptides (i.e., <10 residues); proteins, such as human serum albumin, bovine serum albumin, gelatin or immunoglobulins; and hydrophilic polymers, such as polyvinylpyrrolidone. Stabilizers can be present in an amount of between about 0.1% to about 95%, by weight, or 1% to 75%, taking into account the relative amounts of the other ingredients and components of the hydrogel composition. Additional miscellaneous excipients include bulking agents, (e.g., starch), chelating agents (e.g., EDTA), antioxidants (e.g., ascorbic acid, methionine or vitamin E) and co-solvents.

The dynamic polymeric hydrogel compositions described herein can be present in any suitable material state. For example, the dynamic polymeric hydrogel compositions may be used to stabilize one or more therapeutic agents as an aqueous solution in the form of a hydrogel (i.e., hydrogel solution), or as a dry solid in the form of a powder, produced by, for example, the desiccation, dehydration, evaporation, or lyophilization (i.e., freeze drying) of the aqueous hydrogel solution. A powder composition may be reconstituted or converted to a hydrogel by exposure to an aqueous environment. For example, the addition of a suitable amount of an aqueous solution of a suitable pH (such as a buffer or saline solution) to a lyophilized powder composition will convert the solid-form of the composition to the aqueous hydrogel form of the composition. The material state of the dynamic polymeric hydrogel compositions will depend upon the anticipated environmental stressors (e.g., storage conditions) to which the composition will be exposed, as well as the duration of such exposure. In some embodiments, the dynamic polymeric hydrogel composition is an aqueous solution in the form of a hydrogel while in the presence of one or more environmental stressors. In some embodiments, the dynamic polymeric hydrogel composition is a dry solid in the form of a lyophilized powder while in the presence of one or more environmental stressors.

### Stabilization

The dynamic polymeric hydrogel compositions, which comprise one or more therapeutic agents encapsulated within a hydrogel network of PBA-1,2-diol cross-linked PEG polymer backbones, and, optionally, one or more pharmaceutically acceptable excipients, as described herein, can stabilize the encapsulated therapeutic agents in the presence of environmental stressors. In other words, the encapsulated therapeutic agent(s) of the dynamic polymeric hydrogel compositions can retain up to 99% of its original bioactivity when subjected to a specified condition under which the encapsulated therapeutic agents are transported and/or stored. In some embodiments, the encapsulated therapeutic agent(s) of the dynamic polymeric hydrogel compositions are stabilized when subjected to specified conditions such as, for example, elevated temperatures, humidity, pH changes, and/or the presence of light.

In some embodiments, the encapsulated therapeutic agent(s) of the dynamic polymeric hydrogel composition stored for a period of time under a specified condition (e.g., elevated temperatures, humidity, presence of light, etc.) can retain at least about 20% of its original bioactivity or higher. In some embodiments, the encapsulated therapeutic agent(s) of the dynamic polymeric hydrogel composition stored for a period of time under specified conditions can retain from about 25% to about 99% of its original bioactivity. In some embodiments, the encapsulated therapeutic agent(s) can retain about 30% of its original bioactivity, or about 35%, about 40%, about 45%, about 50%, about 55%, about 60%, about 65%, about 70%, about 75%, about 80%, about 85%, about 90%, about 95%, or about 98% of its original bioactivity. In some embodiments, the encapsulated therapeutic agent(s) can retain can retain at least about 75% of its original bioactivity. In some embodiments, the encapsulated therapeutic agent(s) can retain can retain at least about 80% of its original bioactivity. In some embodiments, the encapsulated therapeutic agent(s) can retain can retain at least about 85% of its original bioactivity. Stated another way, the stability of the encapsulated therapeutic agent(s) of the dynamic polymeric hydrogel composition stored for a period of time under specified conditions can retain (i.e., the ability of an therapeutic agent to retain its original bioactivity can be increased by at least about 35%, at least about 45%, at least about 55%, at least about 65%, at least about 75%, at least about 85%, or at least about 95%, relative to the stability of an un-encapsulated therapeutic agent. In some embodiments, the therapeutic agent can retain at least about 80% of its original bioactivity.

In some embodiments, the encapsulated therapeutic agent(s) within the dynamic polymeric hydrogel composition can be stabilized (i.e., retain at least 50% of its original bioactivity) after being transported and/or stored under specified conditions for any period of time (e.g., hours, days, weeks, months or years). In some embodiments, the encapsulated therapeutic agent(s) of the dynamic polymeric hydrogel composition can be stabilized (i.e., retain at least 50% of its original bioactivity) at a temperature above 0° C. for at least about 3 hours, at least about 6 hours, at least about 9 hours, at least about 12 hours, at least about 24 hours or longer. In some embodiments, the encapsulated therapeutic agent(s) of the dynamic polymeric hydrogel composition can be stabilized for at least about 1 day, at least about 2 days, at least about 3 days, at least about 4 days, at least about 5 days, at least about 6 days, at least about 7 days or longer. In some embodiments, the encapsulated therapeutic agent(s) of the dynamic polymeric hydrogel composition can be stabilized for at least about 1 week, at least about 2 weeks, at least about 3 weeks, at least about 4 weeks or longer. In some embodiments, the encapsulated therapeutic agent(s) of the dynamic polymeric hydrogel composition can be stabilized for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or longer.

Methods for measuring the bioactivity (including original bioactivity) of various therapeutic agents described herein, such as, enzymes, vaccines, proteins, and antibodies, are well known in the art. By way of example, stability or bioactivity of a given encapsulated therapeutic agent of the dynamic polymeric hydrogel composition may be determined based on combinations of time and temperature. For example, stabilization studies can be conducted for 1 to 6 months. Activity assays can be conducted, for example, after 2 weeks, 4 weeks, then monthly. Ranges of temperature, humidity, and/or light exposure can be assessed as different storage conditions. In some embodiments, the resulting bioactivity of the encapsulated therapeutic agent stability tests can be compared with the bioactivity of un-encapsulated therapeutic agents under the same storage conditions. In some embodiments, the resulting bioactivity of the encapsulated therapeutic agent stability tests can be compared with its original bioactivity. In some embodiments, the encapsulated therapeutic agent(s) of the dynamic polymeric hydrogel composition stored for a period of time under specified conditions can retain original bioactivity for at least 5, 10, 20, 30, 40, 50, 60, 70, 80, 90, or 100 times longer than an un-encapsulated therapeutic agent under identical environmental conditions.

The original bioactivity of a therapeutic agent can be measured, for example, within about 20 minutes before or after the therapeutic agent is encapsulated. In some embodiments, the original bioactivity of a therapeutic agent can be measured about 10 seconds to about 20 minutes before or after the therapeutic agent is encapsulated. In some embodiments, the original bioactivity of a therapeutic agent can be measured about 30 seconds before or after the therapeutic agent is encapsulated, or about 1 minute, about 2 minutes, 3, 4, 5, 6, 7, 8, 9, 10, 11, 12, 13, 14, 15, 16, 17, or about 18 minutes before or after the therapeutic agent is encapsulated. In some embodiments, original bioactivity can refer to the bioactivity of a therapeutic agent before the therapeutic agent is encapsulated. In some embodiments, original bioactivity can refer to the maximum bioactivity of the therapeutic (e.g., bioactivity measured immediately after activation of the therapeutic agent via reconstitution or by increasing the temperature). For example, if the encapsulated therapeutic agent is initially in powder, the original bioactivity of the therapeutic agent can be measured immediately after reconstitution. In some embodiments, original bioactivity can refer to bioactivity of an un-encapsulated therapeutic agent when stored or transported under conditions specified by the manufacturer. In some embodiments, original bioactivity refers to the bioactivity of an encapsulated therapeutic agent when stored or transported under conditions specified by the manufacturer.

In some embodiments, the encapsulated therapeutic agent(s) within the dynamic polymeric hydrogel composition can be stored and stabilized (i.e., retain at least 50% of its original bioactivity) at any relative humidity. In some embodiments, the encapsulated therapeutic agent(s) within the dynamic polymeric hydrogel composition can be stored and stabilized at a relative humidity of at least about 5%, at least about 10%, at least about 20%, at least about 30%, at least about 40%, at least about 50% or higher. As used herein, relative humidity is a measurement of the amount of water vapor in a mixture of air and water vapor.

It is generally defined as the partial pressure of water vapor in the air-water mixture, given as a percentage of the saturated vapor pressure under those conditions.

In some embodiments, the encapsulated therapeutic agent(s) within the dynamic polymeric hydrogel composition can be stabilized (i.e., retain at least 50% of its original bioactivity) at any temperature or at a manufacturer's recommended temperature specified for the therapeutic agent. In some embodiments, the compositions can be stored and stabilized in liquid nitrogen or in dry ice. For example, the encapsulated therapeutic agent(s) of the dynamic polymeric hydrogel composition can be stabilized at between about −80° C. and about −20° C., inclusive, or between about −20° C. and about 0° C., inclusive. In some embodiments, the compositions can be stored and stabilized at a temperature above 0° C. For example, the encapsulated therapeutic agent(s) of the dynamic polymeric hydrogel composition can be stabilized at a temperature from about 0° C. to about 100° C. In some embodiments, the encapsulated therapeutic agent(s) of the dynamic polymeric hydrogel composition can be stored and stabilized at a temperature of about 5° C., 15° C., 25° C., 30° C., 35° C., 40° C., 50° C., 55° C., 60° C., 65° C., 70° C., 75° C., 80° C., 85° C., 90° C., or about 95° C. In some embodiments, the encapsulated therapeutic agent(s) of the dynamic polymeric hydrogel composition can be stored and stabilized at temperatures between about 5° C. to about 95° C., about 10° C. to about 90° C., about 20° C. to about 85° C., about 25° C. to about 80° C., about 30° C. to about 75° C., about 35° C. to about 70° C., about 40° C. to about 65° C., or about 50° C. to about 60° C.

In some embodiments, the therapeutic agent retains at least about 35% of its original bioactivity (e.g., at least about 40%, at least about 50%, at least about 60%, at least about 70%, at least about 80%, at least about 90%, at least about 95%) of its original bioactivity or higher activity at about 10° C., at about 25° C., at about 37° C., at about 45° C., at about 50° C. or greater, for at least up to 3 months. In some embodiments, the therapeutic agent retains at least about 10% of the original bioactivity at temperatures of about 37° C. or greater, for at least 4 months. In some embodiments, the hydrogel network of PBA-1,2-diol cross-linked PEG polymer backbones can reduce the degradation rate of the encapsulated therapeutic agent(s) at an elevated temperature (e.g., at least about 25° C., at least about 30° C., at least about 35° C., at least about 40° C., at least about 45° C., at least about 50° C., or higher). Therefore, encapsulated therapeutic agent(s) of the dynamic polymeric hydrogel compositions stored and stabilized at elevated temperatures can have half-lives that are at least about 1.5-fold (e.g., at least about 2-fold, at least about 3-fold, at least about 4-fold, at least about 5-fold, at least about 10-fold, at least about 15-fold, at least about 20-fold, at least about 25-fold, at least about 30-fold, or more) longer than the half-lives of un-encapsulated therapeutic agent(s) that have been stored under the same conditions. As used herein, the term “half-life” refers to the time at which a therapeutic agent retains about 50% of its original.

In some embodiments, the hydrogel network of PBA-1,2-diol cross-linked PEG polymer backbones can encapsulate and stabilize concentrated amounts of therapeutic agents, wherein the hydrogel network prevents aggregation of the encapsulated therapeutic agent molecules and/or decreases the viscosity of the concentrated therapeutic agents. In some embodiments, the hydrogel network of PBA-1,2-diol cross-linked PEG polymer backbones can encapsulate and stabilize concentrated amounts of therapeutic agents, wherein the hydrogel network prevents aggregation of the encapsulated therapeutic agent molecules. In some embodiments, the hydrogel network of PBA-1,2-diol cross-linked PEG polymer backbones can encapsulate and stabilize concentrated amounts of therapeutic agents, wherein the hydrogel network decreases the viscosity of the concentrated therapeutic agents. In general, therapeutic agents lose bioactivity or potency upon aggregation or self-association of therapeutic agent molecules. Thus, therapeutic agents that are susceptible to self-association or self-aggregation can be considered unstable, depending on factors such as formulation preparation procedures and storage conditions. Therapeutic agents known to aggregate or self-associate are biological molecules such as, for example, proteins, peptides, polypeptides, antigens, irnmunogens, enzymes, antibiotics, andbodies or portions thereof (e.g., antibody-like molecules), nucleie acids (e.g., oligonucleotides, polynucleotides, siRNA, shRNA), aptamners, growth factors, hormones, anti-inflammatory agents, vaccines, viruses, viral vectors, and psychotropic agents.

Biological therapeutics, such as, for example, proteins, peptides, antibodies, growth factors, etc., are frequently formulated at high concentrations (i.e., 100 mg/mL to 500 mg/mL, or more) so that the volume of the formulation that must be administered in order to achieve a therapeutically effective dose can be kept small, thereby minimizing patient discomfort. Unfortunately, high concentrations of these biological molecules are more prone to self-aggregation. In addition to having lower bioactivity, formulations of self-associating biological molecules at high concentrations have higher viscosities. Increased viscosities of biological therapeutic formulations make injection delivery by syringe or IV line more difficult or impossible. Depending on the concentration and type of biological molecule, formulations suitable for parenteral injection typically have viscosities from 10 to 30 mPa s.

However, a concentrated formulation of a self-associating biological molecule (e.g., a monoclonal antibody formulation with a concentration ranging from 150 mg/mL to 200 mg/mL) may have a viscosity around 100 mPa s, which would impart parenteral administration difficulties. In some embodiments, the hydrogel network of PBA-1,2-diol cross-linked PEG polymer backbones prevents aggregation and/or decreases viscosity of concentrated formulations of therapeutic agents. In some embodiments, the hydrogel network of PBA-1,2-diol cross-linked PEG polymer backbones prevents aggregation of concentrated formulations of therapeutic agents. In some embodiments, the hydrogel network of PBA-1,2-diol cross-linked PEG polymer backbones decreases viscosity of concentrated formulations of therapeutic agents.

Without wishing to be bound by theory, aggregation of biological molecules is prevented by the polymeric strands of the hydrogel network, which disrupt the non-covalent intermolecular interactions and/or bonds (e.g., hydrogen bonding, disulfide bonds, etc.) formed between the biological molecules, especially in concentrated amounts. Therefore, the hydrogel network of the present invention preserves the bioactivity of concentrated amounts of biological therapeutics by preventing the formation of biological molecule aggregates.

Furthermore, as the polymeric strands of the hydrogel network disrupt and block the intermolecular interactions between biological molecule aggregates, the viscosity of the concentrated biologic therapeutic decreases (e.g., about 100 mPa s without the hydrogel network compared to about 20 mPa s with the hydrogel network), thereby permitting parenteral administration (e.g., intravenous injection, intramuscular injection, subcutaneous injection, intradermal injection, intrathecal injection, epidural injection, intraosseous injection, etc.) of concentrated biological therapeutic formulations in lower doses with ease and at flow rates suitable for subject tolerance.

In some embodiments, the dynamic polymeric hydrogel composition comprises a hydrogel network of PBA-1,2-diol cross-linked PEG polymer backbones and a concentrated amount of a one or more therapeutic agents, wherein the concentrated amount of the one or more therapeutic agents is from about 50 mg/mL to about 2000 mg/mL. In some embodiments, the concentrated amount of the one or more therapeutic agents of the dynamic polymeric hydrogel composition is present in an amount of about 50 mg/mL, 75, 100, 150, 200, 250, 300, 350, 400, 450, 500, 550, 600, 650, 700, 750, 800, 850, 900, 950, 1000, 1250, 1500, 1750, or about 2000 mg/mL. In some embodiments, the concentrated amount of the one or more therapeutic agents of the dynamic polymeric hydrogel composition is present in an amount of from about 50 mg/mL to about 1950 mg/mL, about 75 mg/mL to about 1825 mg/mL, about 100 mg/mL to about 1750 mg/mL, about 150 mg/mL to about 1500 mg/mL, about 200 mg/mL to about 1250 mg/mL, about 250 mg/mL to about 1000 mg/mL, about 300 mg/mL to about 950 mg/mL, about 350 mg/mL to about 900 mg/mL, about 350 mg/mL to about 850 mg/mL, about 400 mg/mL to about 800 mg/mL, about 450 mg/mL to about 750 mg/mL, about 500 mg/mL to about 700 mg/mL, or from about 550 mg/mL to about 650 mg/mL. In some embodiments, the concentrated amount of the one or more therapeutic agents of the dynamic polymeric hydrogel composition is present in an amount of from about 50 mg/mL to about 700 mg/mL, about 75 mg/mL to about 600 mg/mL, about 100 mg/mL to about 500 mg/mL, or from about 150 mg/mL to about 400 mg/mL. In some embodiments, the concentrated amount of the one or more therapeutic agents of the dynamic polymeric hydrogel composition is present in an amount of about 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, or about 600 mg/mL.

In some embodiments, the dynamic polymeric hydrogel composition comprises a hydrogel network of PBA-1,2-diol cross-linked PEG polymer backbones and a concentrated amount of a one or more therapeutic agents, wherein the hydrogel network prevents aggregation of the encapsulated therapeutic agent molecules by at least about 10%, at least about 20%, at least about 30%, at least about 40%, at least about 50%, at least about 60%, at least about 70%, at least about 80%, at least about 90%, at least about 95% or more, as compared to a concentrated amount of the one or more therapeutic agents without the hydrogel network. In some embodiments, the aggregation of the concentrated amount of one or more therapeutic agent molecules encapsulated in the dynamic polymeric hydrogel composition is reduced by at least about 1.5 fold, at least about 2-fold, at least about 3-fold, at least about 4-fold, at least about 5-fold, or more, as compared to a concentrated amount of the one or more therapeutic agents without the hydrogel network. Aggregation or self-association of the encapsulated one or more therapeutic agents can be determined, e.g., by measuring the effective diameter of therapeutic agent particles using dynamic light scattering.

In some embodiments, the dynamic polymeric hydrogel composition comprises a hydrogel network of PBA-1,2-diol cross-linked PEG polymer backbones and a concentrated amount of a one or more therapeutic agents (or formulation thereof), wherein the concentrated amount of the one or more therapeutic agents (or formulation thereof) has a viscosity of from about 5 mPa s to about 75 mPa s. In some embodiments, the concentrated amount of the one or more therapeutic agents of the dynamic polymeric hydrogel composition has a viscosity of about 5 mPa s, 7, 10, 12, 14, 15, 16, 17, 18, 19, 20, 22, 24, 25, 27, 30, 32, 35, 38, 40, 42, 45, 50, 55, 65, or about 75 mPa s. In some embodiments, the concentrated amount of the one or more therapeutic agents of the dynamic polymeric hydrogel composition has a viscosity of from about 5 mPa s to about 70 mPa s, about 7 mPa s to about 65 mPa s, about 10 mPa s to about 55 mPa s, about 12 mPa s to about 50 mPa s, about 14 mPa s to about 45 mPa s, about 15 mPa s to about 40 mPa s, about 16 mPa s to about 42 mPa s, about 17 mPa s to about 40 mPa s, about 18 mPa s to about 38 mPa s, about 19 mPa s to about 35 mPa s, about 20 mPa s to about 32 mPa s, about 22 mPa s to about 30 mPa s, or from about 24 mPa s to about 27 mPa s. In some embodiments, the concentrated amount of the one or more therapeutic agents of the dynamic polymeric hydrogel composition has a viscosity of from about 5 mPa s to about 45 mPa s, about 7 mPa s to about 35 mPa s, about 10 mPa s to about 30 mPa s, or from about 15 mPa s to about 25 mPa s. In some embodiments, the concentrated amount of the one or more therapeutic agents of the dynamic polymeric hydrogel composition has a viscosity of about 10 mPa s, 15 mPa s, 20 mPa s, 25 mPa s, or 30 mPa s.

In some embodiments, the dynamic polymeric hydrogel composition comprises a hydrogel network of PBA-1,2-diol cross-linked PEG polymer backbones and a concentrated amount of a one or more therapeutic agents (or formulation thereof), wherein the hydrogel network decreases the viscosity of the therapeutic agents (or formulation thereof) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75% or more, as compared to a concentrated amount of the one or more therapeutic agents (or formulation thereof) without the hydrogel network. In some embodiments, the viscosity of the concentrated amount of one or more therapeutic agents (or formulation thereof) in the dynamic polymeric hydrogel composition is reduced by at least about 1.5 fold, at least about 2-fold, at least about 3-fold, at least about 4-fold, at least about 5-fold, or more, as compared to a concentrated amount of the one or more therapeutic agents (or formulation thereof) without the hydrogel network. Viscosity of the one or more therapeutic agents (or formulation thereof) can be determined, e.g., using a viscometer. In some embodiments, the dynamic polymeric hydrogel composition comprises a hydrogel network of PBA-1,2-diol cross-linked PEG polymer backbones and a concentrated amount of a one or more therapeutic agents (or formulation thereof), wherein the hydrogel network decreases the viscosity of the encapsulated therapeutic agents or un-encapsulated therapeutic agents. For example, viscosity of concentrated therapeutic agents can be reduced during disassembly of the hydrogel network.

### Release

The one or more stabilized therapeutic agents that have been encapsulated within the dynamic polymeric hydrogel compositions, as described herein, can be released from the hydrogel network upon disassembly of the hydrogel network in response to one or more external stimuli. Non-limiting examples of an external stimulus include, pH change, light irradiation, ionic strength change, exposure to hydrolytic and/or enzymatic activity, and solvent or excipient composition change (e.g., addition of an aqueous diol solution). The disassembly of the hydrogel network can be either irreversible or reversible, depending upon the type of response or change of the dynamic polymeric hydrogel composition that is triggered by the external stimulus. For example, hydrogels can be disassembled irreversibly by the hydrolytic or enzymatic cleavage of the linkers of the PBA-PEG backbone of formula (I) and/or 1,2-diol-PEG backbone of formula (II). Alternatively, the hydrogels can be disassembled reversibly upon the degradation of cross-links between the PBA derivatives and the 1,2-diol moieties.

In some embodiments, the disassembly of the hydrogel network is reversible. Thus, the one or more stabilized therapeutic agents encapsulated within the dynamic polymeric hydrogel compositions, as described herein, can be released or disentangled from the hydrogel network upon the degradation of cross-links between the PBA derivatives and the 1,2-diol moieties in response to one or more external stimuli. In some embodiments, the one or more stabilized therapeutic agents that have been encapsulated are released from the hydrogel network upon the degradation of cross-links between the PBA derivatives and the 1,2-diol moieties in response to one or more external stimuli selected from excipient composition change, pH change, and/or ionic strength change. In some embodiments, the one or more stabilized therapeutic agents that have been encapsulated are released from the hydrogel network upon the degradation of cross-links between the PBA derivatives and the 1,2-diol moieties in response to excipient composition change, pH change, or a combination of both. In some embodiments, the one or more stabilized therapeutic agents that have been encapsulated are released from the hydrogel network upon the degradation of cross-links between the PBA derivatives and the 1,2-diol moieties in response to excipient composition change or pH change.

In some embodiments, the excipient composition change is the addition of a diol-containing solution to the dynamic polymeric hydrogel composition with the encapsulated therapeutic agent. The diol-containing solution can be a solution of any suitable cis-diol molecule that triggers the release of an encapsulated therapeutic agent from the hydrogel network. For example, the diol-containing solution for triggering release of a therapeutic agent(s) can be a solution of cis-diols, such as, for example, citric acid, tris(hydroxymethyl)aminomethane (Tris), polyols (e.g., glycerol, sugar alcohols), sugar molecules, etc. In some embodiments, the excipient composition change is the addition of a polyol solution to the dynamic polymeric hydrogel composition with the encapsulated therapeutic agent. In some embodiments, the excipient composition change is the addition of a sugar solution to the dynamic polymeric hydrogel composition with the encapsulated therapeutic agent. In some embodiments, the pH change is lowering the pH of the dynamic polymeric hydrogel composition with the encapsulated therapeutic agent. In some embodiments, the pH change is increasing the pH of the dynamic polymeric hydrogel composition with the encapsulated therapeutic agent.

In some embodiments, the one or more stabilized therapeutic agents encapsulated in the dynamic polymeric hydrogel composition are released from the hydrogel network upon the addition of a diol-containing solution (e.g., an aqueous sugar solution or a glycerol solution) to the dynamic polymeric hydrogel composition. Without wishing to be bound by theory, the addition of suitable diol-containing solutions to the dynamic polymeric hydrogel compositions leads to the breaking of the boronate ester bonds between the 1,2-diols and PBAs of the modified PEG backbones through competitive displacement by diol molecules of the aqueous diol solution. In some cases, the competitive displacement can be driven by an aqueous diol solution comprising free diol molecules with higher binding affinities to the PBA groups of the PBA-PEG backbones. In other cases, competitive displacement can be driven by the presence of an excess of free diol molecules of the aqueous diol solution, regardless of the binding affinity of the free diol molecule. Thus, addition of the diol-containing solution (e.g., aqueous sugar solution, glycerol solution) to the dynamic polymeric hydrogel composition causes the PBA-1,2-diol cross-links to degrade by competitive replacement of PBA-diol (free) complexes, thereby releasing the one or more stabilized therapeutic agents from the disassembled hydrogel network.

The diol-containing solutions described herein (e.g., aqueous sugar solutions, aqueous glycerol solutions, aqueous solutions of Tris or citric acid, etc.) will be added to the dynamic polymeric hydrogel composition comprising the encapsulated and stabilized therapeutic agent(s) in a concentration sufficient to release the one or more stabilized therapeutic agents from the hydrogel network. Suitable concentrations of the aqueous diol-containing solution can range from 50 μg/mL to 1000 mg/mL. In some embodiments, the concentration of the aqueous diol-containing solution is about 100 μg/mL, 150 μg/mL, 300 g/mL, 500 μg/mL, 1 mg/mL, 5 mg/mL, 10 mg/mL, 50 mg/mL, 75 mg/mL, 100 mg/mL, 200 mg/mL, 300 mg/mL, 400 mg/mL, 500 mg/mL, 600 mg/mL, 700 mg/mL, 800 mg/mL, 900 mg/mL, or about 1000 mg/mL. In some embodiments, the concentration of the aqueous diol-containing solution is from about 50 μg/mL to about 950 mg/mL, about 150 μg/mL to about 900 mg/mL, about 350 μg/mL to about 850 mg/mL, about 600 μg/mL to about 800 mg/mL, about 5 mg/mL to about 750 mg/mL, about 25 mg/mL to about 700 mg/mL, about 50 mg/mL to about 650 mg/mL, about 100 mg/mL to about 600 mg/mL, about 150 mg/mL to about 550 mg/mL, about 200 mg/mL to about 500 mg/mL, about 250 mg/mL to about 450 mg/mL, or from about 300 mg/mL to about 400 mg/mL. In some embodiments, the concentration of the aqueous diol-containing solution is about 100 mg/mL, about 200 mg/mL, about 300 mg/mL, about 400 mg/mL, or about 500 mg/mL.

In some embodiments, the diol-containing solution is an aqueous sugar solution. Any suitable aqueous sugar solution is useful in the methods for releasing the one or more stabilized therapeutic agents encapsulated in the dynamic polymeric hydrogel composition. In the context of the present invention, suitable sugar solutions are those which include saccharide molecules that are capable of (a) displacing the 1,2-diol moiety from the PBA-1,2-diol cross-links and (b) forming a PBA-sugar complex with the PBA group of the PBA-PEG backbone. Suitable sugars, or sugar polymers of one or more sugar molecules, are those which are 1,2-diol-containing saccharides. Therefore, suitable aqueous sugar solutions can include one or more 1,2-diol-containing saccharides or sugar polymers such as, for example, erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, and derivatives thereof. In some embodiments, the aqueous sugar solution is a glucose solution, xylose solution, lyxose solution, dextrose solution, mannose solution, galactose solution, fructose solution, or a lactose solution. In some embodiments, the aqueous sugar solution is a glucose solution, dextrose solution, mannose solution, or fructose solution. In some embodiments, the aqueous sugar solution is a dextrose solution. In some embodiments, the aqueous sugar solution is a fructose solution.

In some embodiments, the one or more stabilized therapeutic agents encapsulated in the dynamic polymeric hydrogel composition are released from the hydrogel network upon lowering the pH of the dynamic polymeric hydrogel composition. As described previously, the pH at which the one or more therapeutic agents are encapsulated within the dynamic polymeric hydrogel network is dependent upon the average of the pKa of the PBA group and the pKa of the 1,2-diol of the modified PEG backbones. (See, for example, Springsteen, G. and Wang, B., Tetrahedron, 2002, 58, 5291-5300.) Therefore, the methods of releasing the stabilized therapeutic agent(s) from the dynamic polymeric hydrogel composition involves lowering or raising the pH of the composition to a pH that is less than or greater than the pKa of the phenylboronic acid group of the PBA modified multi-arm PEG polymer backbone. In this sense, the one or more stabilized therapeutic agents can be released from the dynamic polymeric hydrogel composition when in an acidic or basic environment. For example, a suitable acidic environment can be an acidic buffer solution or an aqueous acid solution, which can be added to the dynamic polymeric hydrogel composition. Suitable acidic environments are those that reverse the PBA-1,2-diol cross-links, caused by shifting the equilibrium of different boronic acid species toward the neutral trivalent phenylboronic acid species. Moreover, a suitable acidic environment will be physiologically acceptable and compatible and will not interfere with the stability of the released therapeutic agents. In some embodiments, the stabilized therapeutic agent(s) can be released from the dynamic polymeric hydrogel composition by lowering the pH of the composition to a pH that is less than the pKa of the PBA group of the PBA-PEG backbone, such as, for example, a pH of less than or equal to about 6.9, 6.5, 6.0, 5.5, 5.0, 4.5, 4.0, 3.5, 3.0, 2.5, 2.0, 1.5, 1.0, or 0.5.

### Administration of Stabilized Therapeutic Agents

After the one or more stabilized therapeutic agent is released from within the network of PBA-1,2-diol cross-linked PEG polymer backbones of the dynamic polymeric hydrogel composition, the stabilized therapeutic agent can be administered to a patient in need thereof. Suitable methods of administration include any which will result in delivery of the stabilized therapeutic agent(s) to the blood stream or directly to the organ, tissue, or site to be treated. In some embodiments, the one or more stabilized therapeutic agent can be administered parenterally, such as injection or infusion, in the form of a solution or suspension. For example, parenteral administration may include administration to a patient intravenously, intradermally, intraperitoneally, intrapleurally, intratracheally, intramuscularly, subcutaneously, subjunctivally, by injection, and by infusion. In some embodiments, the one or more stabilized therapeutic agent can be administered parenterally, wherein the one or more stabilized therapeutic agents are administered in a unit dosage injectable form (solution, suspension, emulsion) as a disassembled dynamic polymeric hydrogel composition. In other words, following the release of the stabilized therapeutic agents upon addition of a sugar solution or pH decrease, the resulting composition can be injected into a patient in need thereof. In some embodiments, the stabilized therapeutic agents are administered in a unit dosage injectable form (solution, suspension, emulsion) after being released from the hydrogel network and separated from the disassembled dynamic polymeric hydrogel composition.

In some embodiments, the stabilized therapeutic agents are administered one time in a therapeutically effective amount as one dosage unit. In some embodiments, a therapeutically effective amount of the stabilized therapeutic agents can be administered as multiple unit dosages, multiple times, for a period of time which will vary depending upon the nature of the particular disorder, its severity, and the overall condition of the subject to whom the stabilized therapeutic agent(s) is administered. For example, administration can be conducted hourly, every 2 hours, three hours, four hours, six hours, eight hours, or twice daily including every 12 hours, or any intervening interval thereof. Administration can be conducted once daily, or once every 36 hours or 48 hours, or once every month or several months. Following treatment, a subject can be monitored for changes in his or her condition and for alleviation of the symptoms of the disorder. The dosage of the stabilized therapeutic agent(s) can either be increased in the event the subject does not respond significantly to a particular dosage level, or the dose can be decreased if an alleviation of the symptoms of the disorder is observed, or if the disorder has been remedied, or if unacceptable side effects are seen with a particular dosage.

In some embodiments, a therapeutically effective amount of a stabilized therapeutic agent(s) can be administered to the subject in a treatment regimen comprising intervals of at least 1 hour, or 6 hours, or 12 hours, or 24 hours, or 36 hours, or 48 hours between dosages. Administration can be conducted at intervals of at least 72, 96, 120, 144, 168, 192, 216, or 240 hours (i.e., 3, 4, 5, 6, 7, 8, 9, or 10 days). In some embodiments, administration of one or more stabilized therapeutic agent is conducted in a chronic fashion over periods ranging from several months to several years. Accordingly, some embodiments of the invention provide a method of treating a disease or condition, wherein the one or more stabilized therapeutic agent is administered to the subject for at least one year. In some embodiments, the one or more stabilized therapeutic agent is administered to the subject for at least 10 years. In some embodiments, the one or more stabilized therapeutic agent is administered to the subject for at least 60 years.

### III. Examples

The following examples are provided by way of illustration only and not by way of limitation. Those of skill will readily recognize a variety of noncritical parameters which could be changed or modified to yield essentially similar results.

### Example 1. Synthesis of the Modified Polymer Backbones

4-Arm-Polyethyleneglycol-NH₂HCl salt (2.0 g, 10 kDa), purchased from JenKem, was dissolved in 100 mL of methanol (MeOH) in a round bottom flask with a magnetic stir bar. D-(+)-Gluconic acid δ-lactone (D-gluconolactone) (0.28 g, 1.6 mmol) was added to the stirring reaction mixture, followed by the addition of triethylamine (2.0 mL, 14.3 mmol). Both the D-gluconolactone and triethylamine were purchased from Sigma-Aldrich. The reaction vessel was covered with foil and allowed to stir continuously for 72 hours at room temperature. The methanol was removed by evaporation and the crude product was then dissolved in deionized (DI) water. The crude product dissolved in DI water was purified by means of dialyses for 72 hours using Regenerated Cellulose Dialysis tubing (MWCO 1 kDa) from Spectrum Labs, and changing the water two times per day. The dialyzed product was lyophilized to a white powder, yielding pure 1,2-diol-PEG having the general structure:

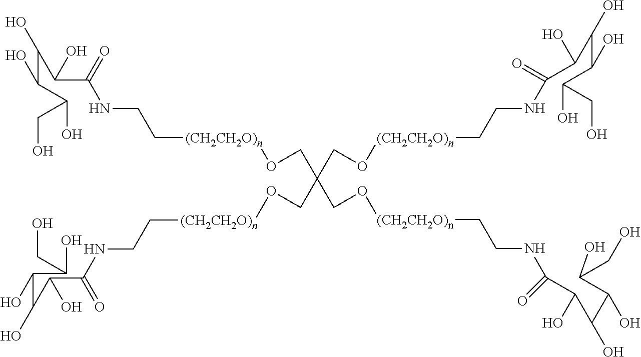

and a molecular weight of about 10,720.64 g/mol (i.e., about 10.7 kDa), wherein subscript n is an integer and the total sum value of the subscripts equal about 220. For example, if each subscript n is 55, the total sum value is 220.

In a separate round bottom flask with a magnetic stir bar, 4-Arm-Polyethyleneglycol-NH₂HCl salt (2.0 g, 10 kDa) was dissolved in 10 mL of methanol (MeOH), followed by the addition of 2-formylphenylboronic acid (APBA) (0.18 g, 1.2 mmol), which was purchased from Sigma-Aldrich. The reaction mixture was purged with argon gas and the reaction flask was sealed with a robber stopper. Triethylamine (0.5 mL, 3.6 mmol) was then added to the stirring reaction mixture through the rubber stopper with a syringe. The reaction was allowed to stir continuously under argon for 72 hours at room temperature. Next, the rubber stopper was removed and the reaction mixture was removed from an argon environment. The reaction flask was placed in an ice water bath and the solution was allowed to cool to 0° C. before the addition of sodium borohydride (NaBH₄) (90 mg, 2.38 mmol), which was purchased from Sigma-Aldrich and slowly added in 10 mg portions directly to the reaction flask. The reaction flask was covered with foil and allowed to stir continuously for 12 hours at room temperature before removing the MeOH by evaporation. The remaining crude product was dissolved in deionized (DI) water and the pH of the solution was balanced to 7.0 using 1 M HCl. Using Regenerated Cellulose Dialysis tubing (MWCO 1 kDa), the crude product was purified by means of dialyses for 72 hours, changing the water two times per day. The dialyzed product was lyophilized to a white powder, yielding pure APBA-PEG having the general structure:

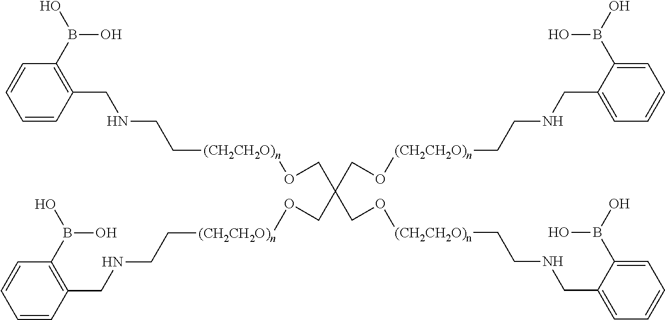

and a molecular weight of about 10,539.8 g/mol (i.e., about 10.5 kDa), wherein subscript n is an integer and the total sum value of the subscripts equal about 220. For example, if each subscript n is 55, the total sum value is 220.

### Example 2. Formation and Shear-Recovery of the Dynamic Polymer Hydrogels

The following example demonstrates the formation of APBA-1,2-diol cross-linked PEG polymer hydrogels and the shear thinning and gel recovery properties thereof. The hydrogel was prepared by combining solutions of the APBA-PEG and 1,2-diol-PEG polymer backbones described in Example 1. To 100 μL of APBA-PEG solution (10.5 kDa, 10 w/v % in 10 mM phosphate PBS buffer, pH=7.4) was added 100 μL of 1,2-diol-PEG solution (10.7 kDa, 10 w/v % in 10 mM phosphate PBS buffer, pH=7.4). The mixture was vortexed until the gel was formed (about 30 s).

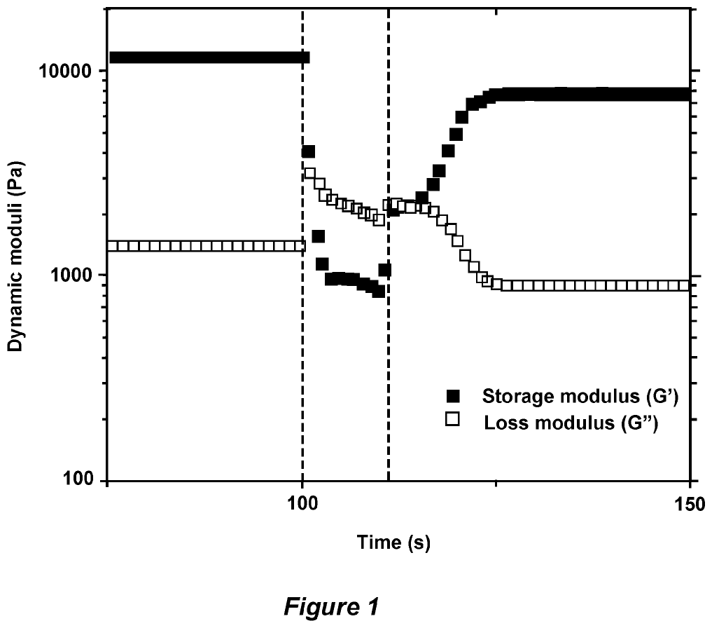

Shear-recovery tests were then performed on the APBA-1,2-diol-PEG hydrogel by placing 200 μL of the 10 w/v % hydrogel sample in a shear rheometer (Anton-Paar MCR 502) using a parallel plate geometry (PP-20 mm, gap setting=0.5 mm, temperature=25° C.). The sample was trimmed, resulting in a hydrogel sample volume of 157 μL between the two plates. After applying an oscillatory shear strain with pre-defined shear strain amplitude (γA) and frequency (ω), the rheometer then measures the frequency-dependent dynamic modulus of the hydrogel material (G*), which is expressed in terms of the storage modulus G′ (i.e., a measure of the stored elastic energy in the material) and the loss modulus G″ (i.e., a representation of the viscous component of the material, or the energy lost and dissipated as heat). When G′>G″, the elastic response dominates and the material can be thought of as exhibiting solid-like behavior with a defined modulus. When G″>G′, the viscous component dominates and the material exhibits liquid-like behavior, such as flow. FIG. 1 shows the results of a three-phase shear-recovery test performed on the boronate ester-based hydrogel, illustrating shear-thinning (viscous flow upon shear) and self-healing (reformation of the gel upon flow cessation) properties of the hydrogel.

The frequency, ω, was kept constant at 10 rad/s during the totality of the shear-recovery experiments. In the first phase, baseline moduli of the hydrogel in its intact gel-like structure were obtained using a low shear strain within the linear viscoelastic region (LVE) of the material. Thus, a shear strain of γA=0.1% was applied for 100 s, resulting in relatively constant storage and loss moduli values of G′=11,500 Pa and G″=1400 Pa, wherein the material exhibited solid-like behavior. In the second phase, a very high shear strain of γA=500% was applied for 10 s, completely disrupting the hydrogel network structure. FIG. 1 shows that this structural disruption resulted in flowing, liquid-like behavior of the material (shear-thinning), in which G″>G′. In order to accommodate the large shear strain, the reversible covalent cross-links between the APBA groups and the 1,2-diol moieties of the modified PEG polymer break, the hydrogel disassembles, and a liquid-like material is observed. In the third phase, time-dependent recovery was measured by reapplying the low shear strain of γA=0.1% for 40 seconds. FIG. 1 shows that the hydrogel material completely recovers, with slightly lower recovery moduli G′=8,000 Pa and G″=900 Pa compared to the moduli of the first phase, which can be attributed to material extrusion during the high shear strain phase. This self-healing behavior was observed due to the reformation of the reversible covalent boronate ester cross-links upon removal of the high shear strain, resulting in solid-like material.

### Example 3. β-Galactosidase Stabilization

The following example demonstrates the encapsulation of β-galactosidase (β-gal) within a dynamic poly(ethylene glycol) (PEG)-based hydrogel. Briefly, 4-arm PEG-amines were functionalized with either PBA derivatives or 1,2-diols to yield APBA-PEG and 1,2-diol-PEG, respectively, as described in Example 1. The shear-thinning and self-healing hydrogel networks containing β-gal and trehalose were prepared by exploiting the reversible covalent cross-linking interactions between the PBA groups and the 1,2-diol moieties. Because these interactions are highly dependent on pH, buffers were used throughout the encapsulation, stabilization, and release processes to control pH. The effect of elevated temperature storage on the stability and biological activity of β-gal encapsulated within APBA-1,2-diol-PEG hydrogels was investigated. Finally, β-gal release kinetics were established using a dextrose solution.

Materials and Equipment: Phosphate buffered saline (PBS, 1×, 10 mM, pH=7.4) prepared according to standard protocols using sodium and potassium phosphate, NaCl, KCl, and adjusting the pH to 7.4 using 1 M HCl or NaOH; Dextrose (Sigma Aldrich, G7528); 4-arm PEG-amine, 10 kDa (JenKem USA, A7011) functionalized according to Example 1 to yield APBA-PEG, 10.5 kDa, lyophilized powder (stored at −20° C.) and 1,2-diol-PEG, 10.7 kDa, lyophilized powder (stored at −20° C.); β-galactosidase (Sigma Aldrich, G5160-25KU); ONPG Substrate Solution from Sigma Aldrich, prepared at 16 mM in PBS (MW: 301.25 g/mol; 4.82 mg/mL); Trehalose (Sigma Aldrich, PHR1344); DI water; Stock solutions: (i) 10 mM 1×PBS, pH=7.4 (store at room temperature), (ii) β-gal solution: 20 mg/mL β-gal and 50 mg/mL trehalose in PBS buffer (aliquot and store at −20° C.), (iii) Release solution: 200 mg/mL dextrose in PBS buffer (store at room temperature), (iv) APBA-PEG solution: 10 w/v % in PBS buffer (prepared fresh), and (v) 1,2-diol-PEG solution: 10 w/v % in PBS buffer (prepare fresh); and Equipment: (i) Plate reader, 420 nm absorbance, (ii) Vortex mixer, (iii) Plate shaker, (iv) Plastic spatulas, (v) Vacuum desiccator, (vi) pH meter, (vii) 2.0 mL tubes (Eppendorf), (viii) 96-well polystyrene microplates (TPP), (ix) P200G, P1000G, P5000G pipettes and P200L multichannel pipettes (Gilson), (x) D200, D1000 and D5000 pipette tips (Gilson), (xi) 50° C. oven.

Procedure: The dynamic polymeric hydrogel composition comprising the encapsulated β-gal was prepared by adding 10 μL of β-gal solution and 15 μL of 1,2-diol-PEG solution to a 2.0 mL Eppendorf tube. The admixture was mixed well using a pipette. To the β-gal-1,2-diol-PEG admixture was added 15 μL of PEG-APBA solution and the mixture was vortexed until the gel was formed (about 30 s). Using a plastic spatula, the gel was moved to the side of the tube and pressed against the side of the tube to form the gel into a 0.5 mm thick disc. The gel was dried for 2 hours in a desiccator under a 100 mbar vacuum. The final 40 μL gel contained 7.5 w/v % PEG, 5.0 mg/mL 3-gal and 12.5 mg/mL trehalose. A positive control solution was prepared by mixing 10 μL of 3-gal solution with 30 μL of PBS in a 2.0 mL Eppendorf tube. Control solutions of a β-gal-APBA-PEG admixture and a β-gal-1,2-diol-PEG admixture were also prepared. The β-gal-APBA-PEG admixture was prepared by adding 10 μL of 3-gal solution, 15 μL of APBA-PEG solution, and 15 μL of PBS buffer to a 2.0 mL Eppendorf tube, and mixing well using a vortex. The β-gal-1,2-diol-PEG admixture was prepared the same way using 10 μL of 3-gal solution, 15 μL of 1,2-diol-PEG solution, and 15 μL of PBS buffer.

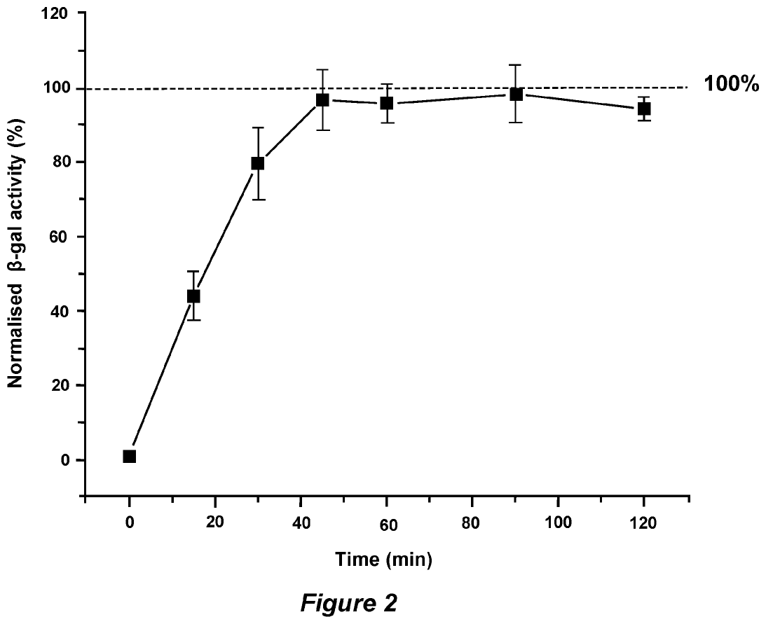

The β-gal-containing gel (Sample 1—“Gel-encapsulated β-gal”), the positive control β-gal solution (Sample 2—“Non-encapsulated β-gal”), the control β-gal-APBA-PEG admixture (Sample 3—“β-gal-APBA-PEG only”), and the control β-gal-1,2-diol-PEG admixture (Sample 4—“β-gal-1,2-diol-PEG only”) were then sealed and placed in the 50° C. oven for 3 days. After allowing the samples to cool to room temperature, 1960 μL of release solution (200 mg/mL dextrose) was added to each Eppendorf tube (Samples 1, 2, 3, and 4), thereby initiating β-gal release for the β-gal-containing gel (Sample 1—“Gel-encapsulated β-gal”). The tubes were placed on a plate shaker and gently mixed for at least 1 hour, ensuring that the gels were fully dissolved and the solutions fully mixed, resulting in β-gal concentrations of 100 μg/mL for Samples 1, 2, 3, and 4. β-gal release kinetics had been monitored previously for a separate β-gal-containing gel sample, immediately before and after the addition of 200 mg/mL dextrose for 120 minutes. As shown in FIG. 2, 100% of β-gal was released from encapsulation within 40 minutes after the addition of dextrose. Hydrogel network dissolution was induced by the addition of excess water-soluble guest molecules (i.e., dextrose), which displaced the 1,2-diol-PEG moiety of the APBA-1,2-diol-PEG cross-linking interaction, causing β-gal release.

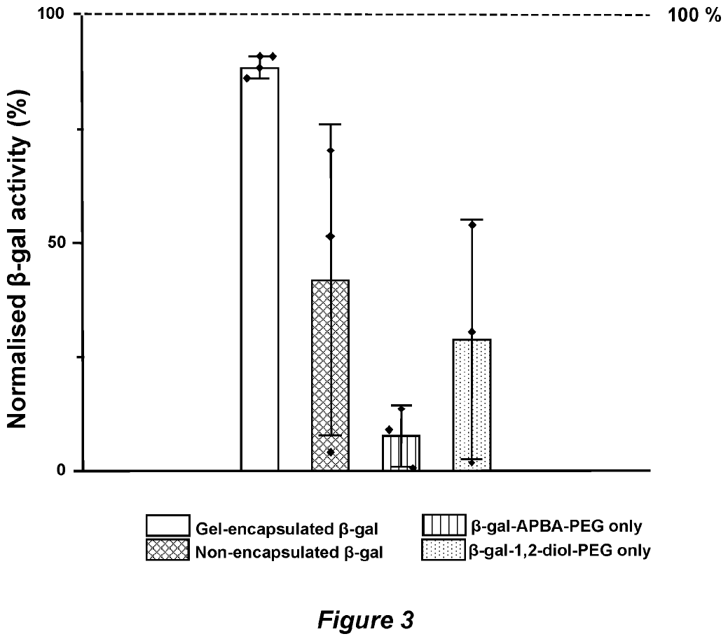

After an hour of mixing the Samples 1, 2, 3, and 4 containing 100 μg/mL β-gal, 100 μL of each sample was transferred to a well in a 96-well microplate. Using a multichannel pipette, 100 μL of ONPG (16 mM) substrate solution was added to each well, ensuring that the ONPG solution was at room temperature before using. The mean absorbance was recorded at 420 nm every 60 s for 10 min. The kinetic slope recorded for each well was proportional to the mean β-gal activity. β-gal activity was normalized with freshly prepared 100 μg/mL β-gal solution, ensuring that the β-gal solution was taken from the same stock as the gel and controls. As shown in FIG. 3, only about 40% of the enzymatic activity was retained for the non-encapsulated β-gal sample (Sample 2), whereas about 90% of the enzymatic activity was retained for the encapsulated β-gal sample (Sample 1). Only about 10% activity was retained in the β-gal-APBA-PEG admixture (Sample 3) and about 40% activity was retained in the β-gal-1,2-diol-PEG admixture (Sample 4).

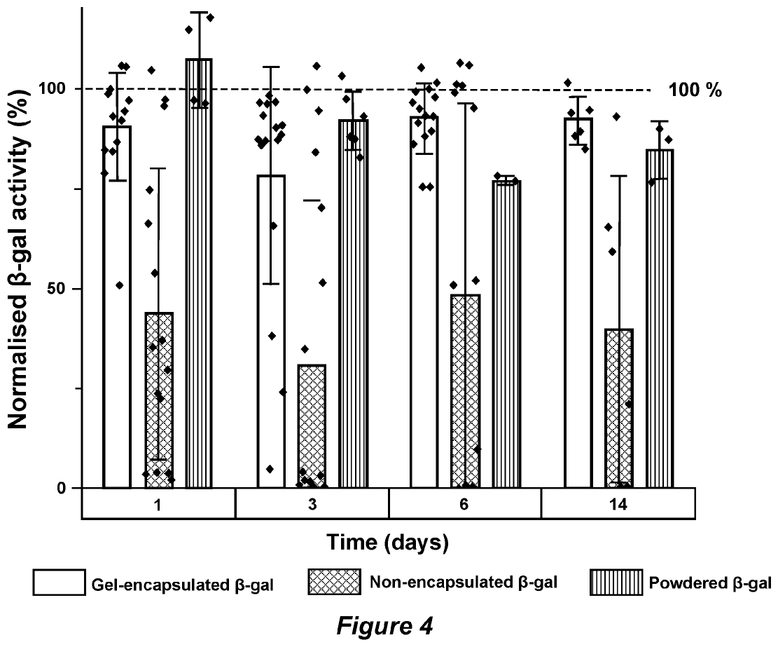

In a similar experiment, β-gal-containing gels (Sample 1A—“Gel-encapsulated β-gal”), positive control β-gal solutions (Sample 2A—“Non-encapsulated β-gal”), and lyophilized powders of the as-shipped β-gal composition, purchased from Sigma-Aldrich (Sample 3A—“Powdered β-gal”) were sealed and placed in the 50° C. oven for 1 day, 3 days, 6 days, and 14 days. As described previously, 1960 μL of release solution was added to each sample (1A, 2A, and 3A) stored for 1 day, 3 days, 6 days, and 14 days at 50° C., initiating β-gal release for the 1A samples. Each sample was treated with substrate solutions and analyzed for enzymatic activity (see above). FIG. 4 shows the results of β-gal stabilization after 1 day, 3 days, 6 days, and 14 days of storage at 50° C. Gel-encapsulated β-gal retained up to 90% enzymatic activity, even after 14 days of storage at 50° C. (Sample 1A—“Gel-encapsulated β-gal”), and the lyophilized as-shipped β-gal powder (Sample 3A—“Powdered β-gal”) retained about 80% enzymatic activity (FIG. 4). As shown in FIG. 4, only about 40% of the enzymatic activity was retained for the non-encapsulated β-gal sample (Sample 2A).

### Example 4. Topoisomerase Stabilization

The following example demonstrates the use of a dynamic polymeric hydrogel composition of the invention to encapsulate and stabilize topoisomerases, DNA gyrase (Cat. # TG2000G, purchased from TopoGen, Inc.) and topoisomerase IV (TopIV, Cat. # TG1007, purchased from TopoGen, Inc.), under various storage conditions. Samples of 80 U/μL DNA gyrase and TopIV encapsulated within the APBA-1,2-diol-PEG hydrogel were prepared and dried as described above in Example 3, except that volumes used were reduced by 10-fold resulting in a final gel volume of 4 μL. Non-encapsulated 80 U/μL DNA gyrase and TopIV samples were suspended in buffers that came with the enzyme as purchased (see Example 3). The gel-encapsulated DNA gyrase and TopIV samples and the non-encapsulated DNA gyrase and TopIV samples were stored at 50° C. for 1 hour. After 1 hour of storage at 50° C., V_{f}=40 μL of a glycerol-containing buffer release solution (provided by TopoGen, Inc.) was added to each enzyme sample, thereby initiating DNA gyrase and TopIV release for the gel-encapsulated samples. The sample tubes were placed at room temperature for 30 minutes and gently mixed by vortexing every 10 minutes, ensuring that the gels were fully dissolved and the solutions completely mixed. This resulted in DNA gyrase and TopIV concentrations of 2 U/μL.

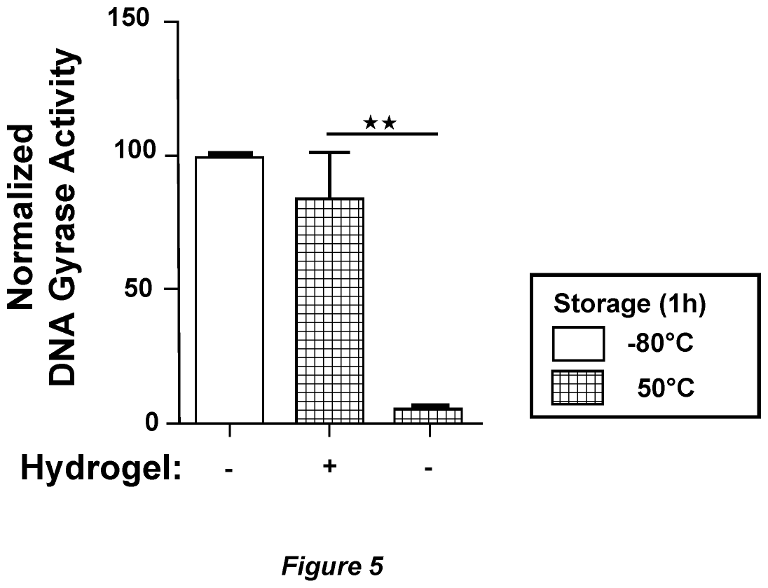

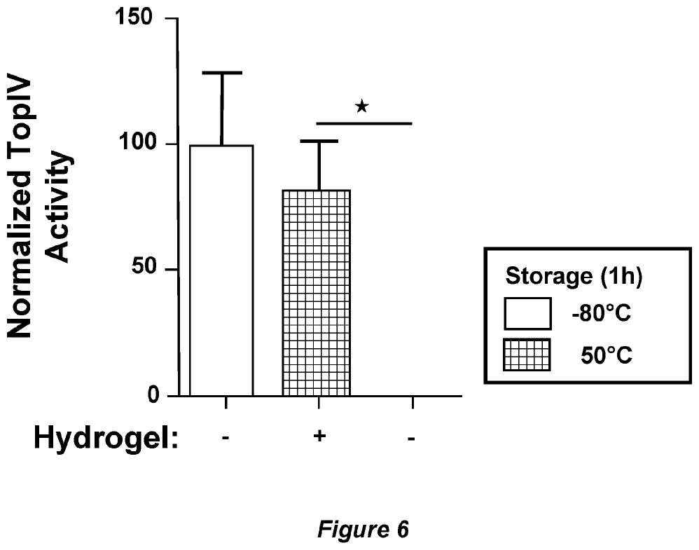

Temperature protection of the topoisomerases was verified by activity assays. DNA gyrase activity was assayed via a DNA supercoiling assay, wherein relaxed plasmid DNA is converted to its supercoiled form by active DNA gyrase. TopIV activity was assayed by a decatenation assay, where catenated (interlocking) kinetoplast DNA (kDNA) is broken down into circular decatenated kDNA monomers by active TopIV. The activity of the stressed enzymes (storage at 50° C. for 1 hour) was compared to the activity of fresh topoisomerases stored at −80° C. in equal concentrations. As shown in FIG. 5, only about 5% of the enzymatic activity was retained for the non-encapsulated DNA gyrase sample, whereas about 80% of the enzymatic activity was retained for the encapsulated DNA gyrase sample. The results shown in FIG. 6 indicate that there was no enzymatic activity retained for the non-encapsulated TopIV sample, but about 80% enzymatic activity was retained for the encapsulated TopIV sample.

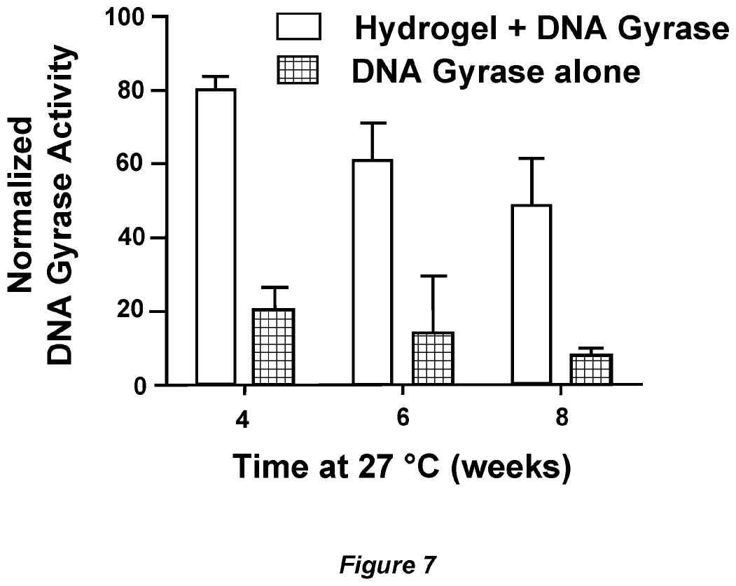

In a similar experiment, samples of DNA gyrase-containing gels and non-encapsulated DNA gyrase solutions were stored at 27° C. oven for 4, 6, and 8 weeks. As described previously, 40 μL of glycerol release solution was added to each DNA gyrase sample stored for 4, 6, and 8 weeks at 27° C., initiating DNA gyrase release for the encapsulated samples. Each sample was analyzed for enzymatic activity using the DNA supercoiling assay described above. The activity of the stressed enzymes was normalized to enzymes freshly prepared from stocks stored at −80° C. (100%). FIG. 7 shows the results of DNA gyrase stabilization after 4, 6, and 8 weeks of storage at 27° C. Gel-encapsulated DNA gyrase retained 80% enzymatic activity after 4 weeks of storage, over 60% activity after 6 weeks, and about 50% enzymatic activity after 8 weeks of storage. However, only about 20% of enzymatic activity was retained for the non-encapsulated DNA gyrase sample after 4 weeks of storage, about 15% after 6 weeks, and only about 8% after 8 weeks of storage at 27° C.

### Example 5. Anti-Human TNFα Monoclonal Antibody Stabilization

The following example demonstrates the use of a dynamic polymeric hydrogel composition of the invention to encapsulate and stabilize monoclonal antibody (mAB) HUMIRA® (adalimumab, Creative Biolabs) under various storage conditions. Samples of 40 μL (5.0 mg/mL) adalimumab were encapsulated within the APBA-1,2-diol-PEG hydrogel (as described previously) and were either vacuum dried or not dried. Non-encapsulated adalimumab samples were prepared by suspending the mAB (5.0 mg/mL) in 40 μL PBS buffer, and either vacuum dried or not dried. The dried and not dried samples of the encapsulated and non-encapsulated adalimumab were incubated at 65° C. or 4° C. for 24 hours. After incubation, all samples were treated with 360 μL release buffer (500 mg/mL dextrose, PBS) shaking at 200 rpm RT for 1 hr and then diluted to 64 ng/mL in 400 μL DMEM (Invitrogen) also containing 1024 pg/mL human TNFα (Peprotech) DMEM containing human TNFα. 100 μL of each diluted mAB-DMEM sample was transferred to a well in a 96-well plate containing HEK-Dual-TNFα reporter cells containing an NFKB-SEAP (Secreted Alkaline Phosphatase) reporter gene (Human and Murine TNF-α SEAP/Lucia, unit size: 3-7×10⁶ cells, InvivoGen). Using the manufacturer's protocol, the cells were cultured for 24 hours and the SEAP supernatant was measured to determine temperature protection of the mAB. Data was normalized to unstressed (4° C. incubation) PBS formulated samples.

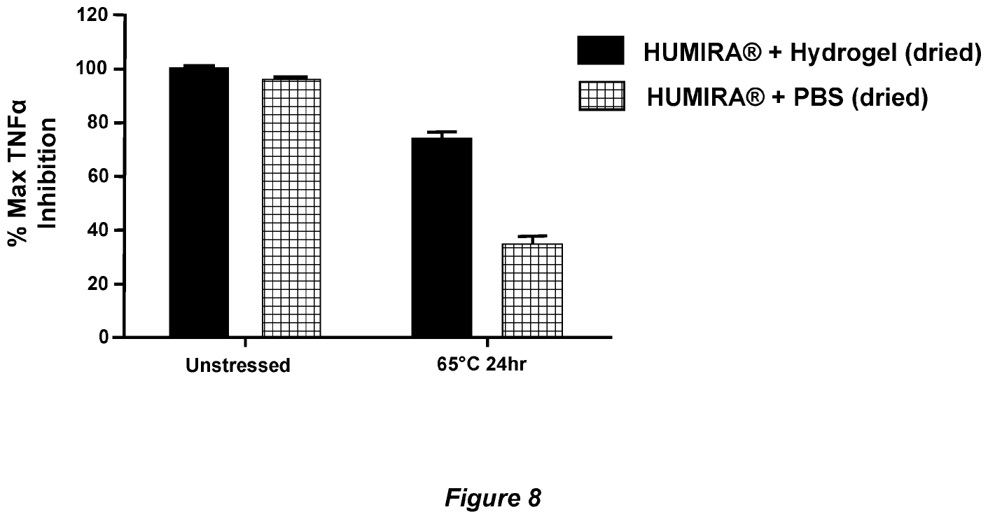

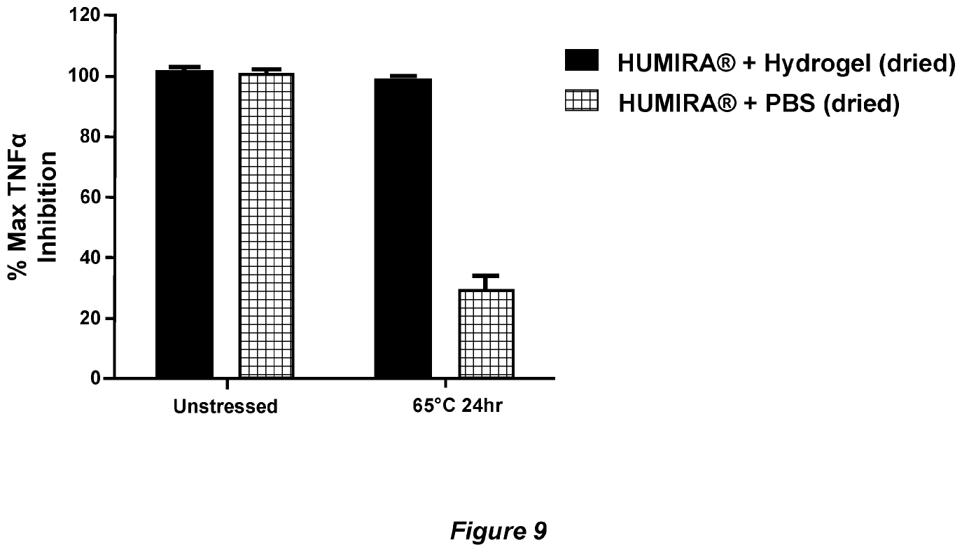

As shown in FIG. 8, the dried encapsulated HUMIRA® sample reached about 80% TNFα inhibition after incubating for 24 hours at 65° C. The dried non-encapsulated HUMIRA® sample only reached about 35% TNFα inhibition after incubating for 24 hours at 65° C. (FIG. 8). The results shown in FIG. 9 indicate that the not dried encapsulated HUMIRA® sample retained almost 100% TNFα inhibition functionality, while the not dried non-encapsulated HUMIRA® sample was only able to reach about 30% TNFα inhibition.

### Example 6. Adenovirus Stabilization

The following example demonstrates the use of a dynamic polymeric hydrogel composition of the instant invention to encapsulate and stabilize adenovirus type 5 under various storage conditions. Samples of 40 μL (5.0 mg/mL) adenovirus type 5 containing CMV-GFP cassette (Ad5-GFP, made in house), were formulated either within the APBA-1,2-diol-PEG hydrogel (encapsulated) or in PBS buffer (non-encapsulated), and then either vacuum dried or not dried, as described previously in Example 5. Dried and not dried samples of the encapsulated and non-encapsulated Ad5-GFP were stored for 4 hours at room temperature (25° C.-27° C.). Not dried samples of the adenovirus (encapsulated and non-encapsulated) were also subjected to freeze/thaw conditions (−20° C. 4 hr, room temp thaw lhr), in which the samples were subjected to 5 freeze/thaw cycles. After completion of the stress tests, all samples were treated with 360 μL release buffer (500 mg/mL dextrose, PBS) for 1 hr shaking at 200 rpm at RT and then diluted to 4×10⁶ virus particles per ml into 1 mL DMEM media. The entire 1 mL of Ad5-GFP-DMEM sample was applied to 400,000 HEK-293 (obtained from ATCC) cells in 12-well plates for a final multiplicity of infection (MOI) of 10. A control sample of uninfected HEK-293 cells was also prepared. After transduction, the cells cultures were incubated for 48 hours. The cells were then trypsonized and resuspended in DMEM media and cellular GFP fluorescence was measured using a Beckman Cytoflex Flow Cytometry Analyzer. GFP was normalized to cells infected with fresh Ad5-GFP MOI=10.

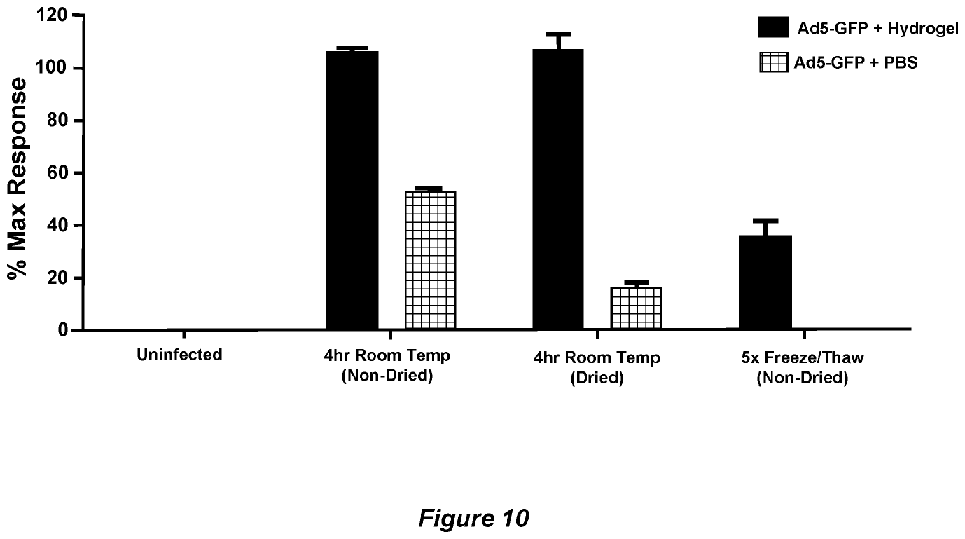

As shown in FIG. 10, the dried and not dried encapsulated Ad5-GFP samples maintained 100% infectivity after being stored for 4 hours at room temperature. The not dried and dried non-encapsulated Ad5-GFP samples possessed about 50% and about 20% infectivity, respectively, after 4 hours of storage at room temperature (FIG. 10). The results of FIG. 10 also show that the not dried encapsulated Ad5-GFP sample retained over 30% infectivity after 5 freeze/thaw cycles, while the not dried non-encapsulated Ad5-GFP sample showed no activity after being subjected to the freeze/thaw conditions.

### Example 7. Recombinant Human TNFα Stabilization

The following example demonstrated the use of a dynamic polymeric hydrogel composition of the invention to encapsulate and stabilize pro-inflammatory cytokine recombinant human TNFα (Peprotech) under various storage conditions. Samples of 40 μL (5.0 mg/mL) TNFα were either encapsulated within the APBA-1,2-diol-PEG hydrogel or suspended in PBS buffer (as described previously) and vacuum dried. Encapsulated and non-encapsulated TNFα samples were stored at 4° C. for 3 days, room temperature for 3 days, 37° C. for 3 days, or subjected to 5 freeze/thaw cycles. Following the completion of the stress tests, all samples were treated with 360 μL release buffer (200 mg/mL fructose, PBS) for 10 minutes shaking at 200 rpm and then diluted to 4096 pg/mL into 200 μL DMEM media. 50 μL of Each DMEM-diluted TNFα sample was applied to 96-well plates containing 150 μL HEK-Dual-TNF reporter cells (Invivogen cat# hkd-tnfa) containing an NFKB-SEAP reporter gene. Using the manufacturer's protocol, the cells were cultured for 24 hours and the SEAP supernatant was measured to determine temperature protection of TNFα. Data was normalized to fresh unstressed TNFα.

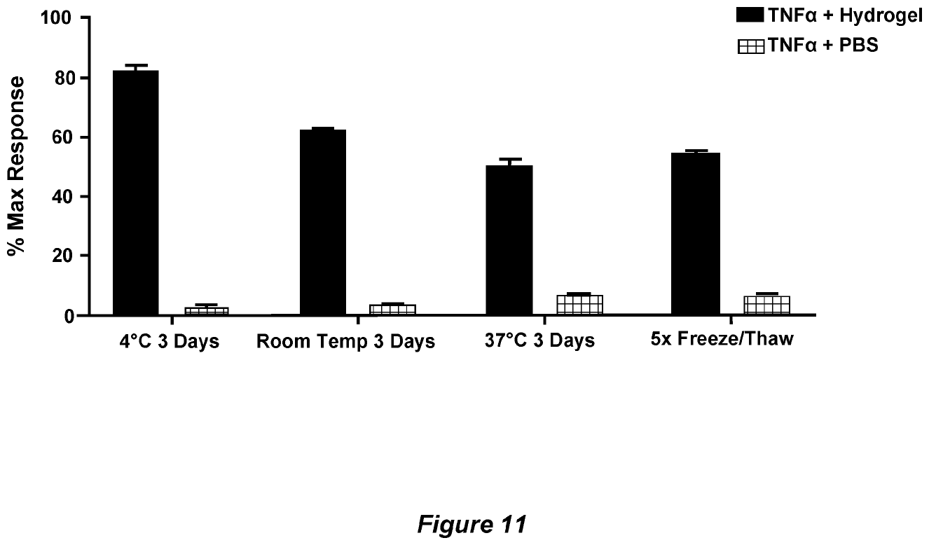

As shown in FIG. 11, after being stored for 3 days at 4° C., the encapsulated TNFα sample retained over 80% activity and the non-encapsulated TNFα sample retained less than 5% activity. After 3 days of storage at room temperature, the encapsulated TNFα sample retained over 60% activity compared to only about 5% activity for the non-encapsulated TNFα sample. For the TNFα samples stored at 37° C. for 3 days, the gel-encapsulated TNFα sample retained about 50% activity, while only about 5-10% activity was retained for the non-encapsulated TNFα sample. FIG. 11 also shows that the encapsulated TNFα sample retained about 60% activity after 5 freeze/thaw cycles and the non-encapsulated TNFα sample retained only about 5-10% activity.

### Example 8. Recombinant Human IL-12 Stabilization

The following example demonstrated the use of a dynamic polymeric hydrogel composition of the invention to encapsulate and stabilize heterodimeric cytokine recombinant human IL-12 (Peprotech) under various storage conditions. Samples of 40 μL (5.0 mg/mL) IL-12 were either encapsulated within the APBA-1,2-diol-PEG hydrogel or suspended in PBS buffer (as described previously) and vacuum dried. Encapsulated and non-encapsulated IL-12 samples were stored at 4° C. for 3 days, room temperature for 3 days, 37° C. for 3 days, or subjected to 5 freeze/thaw cycles. Following the completion of the stress tests, all samples were treated with 360 μL release buffer (200 mg/mL fructose, PBS) for 10 minutes shaking at 200 rpm and then diluted to 400 ng/mL into 200 μL DMEM media. 50 μL of each DMEM-diluted IL-12 sample was applied to 96-well plates containing 150 μL HEK-Blue-IL12 reporter cells (Invivogen cat# hkb-IL12) containing a STAT4-SEAP reporter gene. Using the manufacturer's protocol, the cells were cultured for 24 hours and the SEAP supernatant was measured to determine temperature protection of IL-12. Data was normalized to fresh unstressed IL-12.

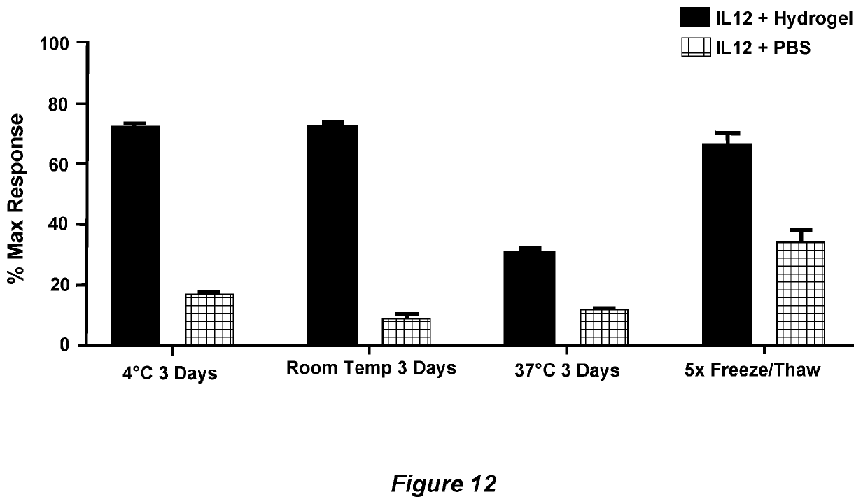

As shown in FIG. 12, after being stored for 3 days at 4° C., the encapsulated IL-12 sample retained about 70% activity and the non-encapsulated IL-12 sample retained less than 20% activity. After 3 days of storage at room temperature, the encapsulated IL-12 sample retained over 70% activity compared to less than 10% activity for the non-encapsulated IL-12 sample. For the IL-12 samples stored at 37° C. for 3 days, the gel-encapsulated IL-12 sample retained about 30% activity, while about 10% activity was retained for the non-encapsulated IL-12 sample. FIG. 12 also shows that the encapsulated IL-12 sample retained over 60% activity after 5 freeze/thaw cycles and the non-encapsulated IL-12 sample retained about 35% activity.

### Example 9. ALP Encapsulation, Stabilization, and Release

The following example demonstrates the encapsulation of alkaline phosphatase (ALP) within a dynamic poly(ethylene glycol) (PEG)-based hydrogel. Briefly, 4-arm PEG-amines are functionalized with either PBA derivatives or 1,2-diols to yield APBA-PEG and 1,2-diol-PEG, respectively, as described in Example 1. The shear-thinning and self-healing hydrogel networks containing ALP and trehalose are prepared by exploiting the reversible covalent cross-linking interactions between the PBA groups and the 1,2-diol moieties. Because these interactions are highly dependent on pH, buffers will be used throughout the encapsulation, stabilization, and release processes to control pH. Finally, the ALP is released from the dynamic polymer hydrogel network using a dextrose solution, which disrupts the hydrogel network by competitively binding to the APBA end groups.

Materials and Equipment: Trizma® base (Sigma Aldrich, T1503); Trizma® HCl (Sigma Aldrich, T3253); Dextrose (Sigma Aldrich, G7528); 4-arm PEG-amine, 10 kDa (JenKem USA, A7011) functionalized according to Example 1 to yield APBA-PEG, 10.5 kDa, lyophilized powder (stored at −20° C.) and 1,2-diol-PEG, 10.7 kDa, lyophilized powder (stored at −20° C.); Alkaline Phosphatase (Sigma Aldrich, P7640); 1-Step PNPP Substrate Solution (Thermo Fischer Scientific, 37621); Trehalose (Sigma Aldrich, PHR1344); DI water; Stock solutions: (i) 100 mM Tris buffer: Combine 13.22 g/L Trizma® HCl and 1.94 g/L Trizma® base in DI water and adjust the pH to 7.4 with 1 M HCl (store at room temperature), (ii) ALP solution: 10 mg/mL ALP and 25 mg/mL trehalose in Tris buffer (aliquot and store at −20° C.), (iii) Release solution: 100 mg/mL dextrose in Tris buffer (store at room temperature), (iv) APBA-PEG solution: 10 w/v % in Tris buffer (prepared fresh), and (v) 1,2-diol-PEG solution: 10 w/v % in Tris buffer (prepare fresh); and Equipment: (i) Plate reader, 405 nm absorbance, (ii) Vortex mixer, (iii) Plate shaker, (iv) Plastic spatulas, (v) Vacuum desiccator, (vi) pH meter, (vii) 2.0 mL tubes (Eppendorf), (viii) 96-well polystyrene microplates (TPP), (ix) P200G, P1000G, P5000G pipettes and P200L multichannel pipettes (Gilson), (x) D200, D1000 and D5000 pipette tips (Gilson).

Procedure: The dynamic polymeric hydrogel composition comprising the encapsulated ALP is prepared by adding 10 μL of ALP solution and 15 μL of 1,2-diol-PEG solution to a 2.0 mL Eppendorf tube. The admixture is mixed well using a vortex. To the ALP-1,2-diol-PEG admixture is added 15 μL of PEG-APBA solution and the mixture is vortexed until the gel is formed (about 30 s). Using a plastic spatula, the gel is moved to the side of the tube and pressed against the side of the tube to form the gel into a 0.5 mm thick disc. The gel is dried for 2 hours in a desiccator under a 100 mbar vacuum. The final 40 μL gel contains 7.5 w/v % PEG, 2.5 mg/mL ALP and 6.25 mg/mL trehalose. Another dynamic polymeric hydrogel composition is prepared as the negative control gel, following the gelation steps described above and replacing the 10 μL of ALP solution with 10 μL of Tris Buffer. A positive control solution is also prepared by mixing 10 μL of the ALP solution with 30 μL of Tris buffer in a 2.0 mL Eppendorf tube.

The ALP-containing gels (Sample 1), the negative control gels (Sample 2), and the positive control solutions (Sample 3) are then placed in the 50° C. oven for 1 day, 3 days, and 6 days. Next, 1960 μL of release solution (100 mg/mL dextrose) will be added to each Eppendorf tube (Samples 1, 2, and 3), thereby initiating ALP release. The tubes will be placed on a plate shaker and gently mixed for at least 1 hour, ensuring that the gels will be fully dissolved and the solutions fully mixed. The addition of the release solution will result in ALP concentrations of 50 μg/mL for Samples 1 and 3 (Sample 2 does not contain ALP—negative control gel). A 10 μL aliquot will be transferred from each sample to a well in a 96-well microplate, followed by the addition of 90 μL of Tris buffer to each well (final ALP concentration in the wells=5 μg/mL). Using a multichannel pipette, 100 μL of PNPP substrate solution will be added to each well, ensuring that the PNPP solution is at room temperature before using. The mean absorbance will be recorded at 405 nm every 60 s for 10 min. The kinetic slope recorded for each well is proportional to the mean ALP activity. ALP activity will be normalized with freshly prepared 5 μg/mL ALP solution, ensuring that the ALP solution is taken from the same stock as the gel and controls. Even after 6 days of storage at 50° C., the gel-encapsulated ALP (Sample 1) will retain up to 85% activity (or more). The ALP of the positive control solution (Sample 3) will not retain as much activity (50% or less) because the ALP of these samples was not encapsulated by the hydrogel networks during storage at 50° C. for 1 day, 3 days, and 6 days.

1. A method for stabilizing a therapeutic agent comprising encapsulating the therapeutic agent in a dynamic polymeric hydrogel composition, the dynamic polymeric hydrogel composition comprising a combination of:
a phenylboronic acid (PBA) modified multi-arm polyethylene glycol (PEG) polymer backbone of formula (I) and a 1,2-diol modified multi-arm polyethylene glycol (PEG) polymer backbone of formula (II): 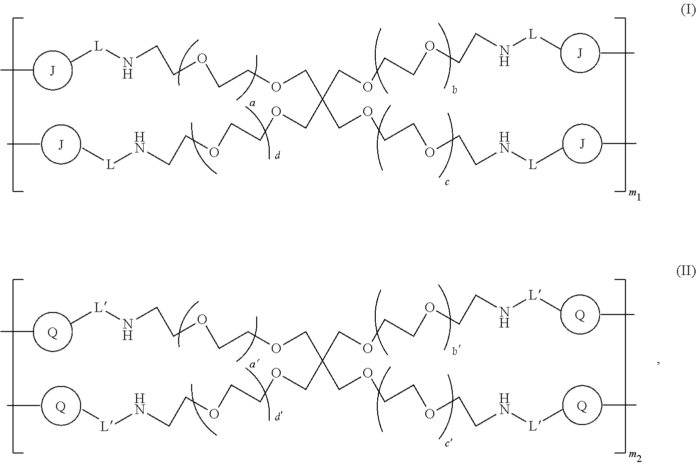
wherein
subscripts a, a′, b, b′, c, c′, d, and d′ are each independently an integer selected from 1 to 500;
subscripts m₁ and m₂ are each independently an integer selected from 10 to 20,000;
linkers L and L′ are each independently selected from a bond, —C(O)—, substituted or unsubstituted alkylene, substituted or unsubstituted —C(O)-alkylene, and substituted or unsubstituted heteroalkylene;
each J is a phenylboronic acid derivative; and
each Q is a 1,2-diol moiety; and
wherein the PBA modified multi-arm PEG polymer backbone and the 1,2-diol modified multi-arm PEG polymer backbone are reversibly covalently cross-linked through the phenylboronic acid derivatives and the 1,2-diol groups.

2. The method of claim 1, wherein
subscripts a, a′, b, b′, c, c′, d, and d′ are each independently an integer selected from 10 to 250;
subscripts m₁ and m₂ are each independently an integer selected from 25 to 10,000;
each linker L is selected from a bond, —C(O)—, substituted or unsubstituted C₁₋₆ alkylene, and unsubstituted —C(O)—C₁₋₆ alkylene, wherein substituted C₁₋₆ alkylene is substituted with at least one substituent selected from —OH, —NH₂, —SH, —CN, —CF₃, —COOH, —C(O)NH₂, halogen, unsubstituted C₁₋₃ alkyl, and substituted C₁₋₃ alkyl; and
each linker L′ is selected from a bond, —C(O)—, unsubstituted C₁₋₆ alkylene, and unsubstituted —C(O)—C₁₋₆ alkylene.

3. The method of claim 1, wherein the phenylboronic acid derivative of the PBA modified multi-arm PEG polymer backbone comprises a phenylboronic acid group of formula (III): 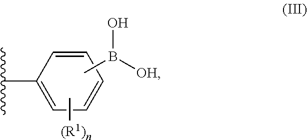
wherein
each R¹ is each independently selected from the group consisting of substituted or unsubstituted alkyl, substituted or unsubstituted heteroalkyl, halogen, —CN, —OH, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a};
R^{a} and R^{b} are each independently selected from the group consisting of hydrogen and C₁₋₆ alkyl; and
subscript n is an integer from 0-4;
wherein the pKa of the phenylboronic acid group is less than 7.8.

4. The method of claim 3, wherein
each R¹ is each independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b};
R^{a} and R^{b} are each independently selected from the group consisting of hydrogen and C₁₋₆ alkyl; and
subscript n is an integer from 0-3;
wherein the pKa of the phenylboronic acid group is between about 3.5 and about 7.4.

5. The method of claim 1, wherein the phenylboronic acid derivative of the PBA modified multi-arm PEG polymer backbone comprises a phenylboronic acid group of formula (IIIA) or formula (IIIB): 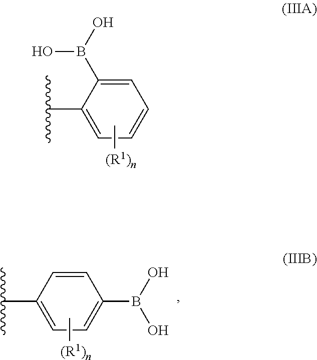
wherein
each R¹ is each independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NR^{a}R^{b}, —CH₂NR^{a}R^{b}, —C(O)R^{a}, and —C(O)OR^{a};
R^{a} and R^{b} are each independently selected from the group consisting of hydrogen and C₁₋₃ alkyl; and
subscript n is an integer from 0-2;
wherein the pKa of the phenylboronic acid group is between about 4.0 and about 7.2.

6. The method of claim 1, wherein the 1,2-diol moiety of the 1,2-diol modified multi-arm PEG polymer backbone comprises a 1,2-cis-diol group of formula (IV): 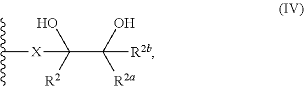
wherein
R², R^{2a}, and R^{2b} are each independently selected from the group consisting of hydrogen, substituted or unsubstituted alkyl, substituted or unsubstituted heteroalkyl, halogen, —CN, —OH, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a};
wherein, optionally, R² and one of R^{2a} or R^{2b} are combined together to form a substituted or unsubstituted cycloalkyl, substituted or unsubstituted heterocycloalkyl, substituted or unsubstituted aryl, or substituted or unsubstituted heteroaryl;
R^{a} and R^{b} are each independently selected from the group consisting of hydrogen and C₁₋₆ alkyl; and
X is selected from the group consisting of a bond, substituted or unsubstituted alkylene, substituted or unsubstituted heteroalkylene, and —C(O)—.

7. The method of claim 6, wherein
R², R^{2a}, and R^{2b} are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, —NR^{a}R^{b}, —CH₂NR^{a}R^{b}, —C(O)R^{a}, —C(O)OR^{a}, and —C(O)NR^{a}R^{b};
wherein, optionally, R² and one of R²a or R^{2b} are combined together to form a substituted or unsubstituted C₃₋₇ cycloalkyl, or substituted or unsubstituted 3 to 7 membered heterocycloalkyl;
R^{a} and R^{b} are each independently selected from the group consisting of hydrogen and C₁₋₆ alkyl; and
X is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—.

8. The method of claim 1, wherein the 1,2-diol moiety of the 1,2-diol modified multi-arm PEG polymer backbone comprises a 1,2-cis-diol group of formula (IVA): 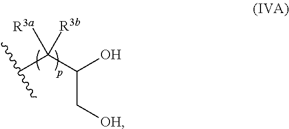
wherein
R^{3a} and R^{3b} are each independently selected from the group consisting of hydrogen, substituted or unsubstituted C₁₋₃ alkyl, methoxy, ethoxy, fluoro, chloro, bromo, iodo, —OH, —NR^{a}R^{b}, —CH₂NR^{a}R^{b}, —C(O)R^{a}, and —C(O)OR^{a};
wherein, optionally, one or more of R³a and R^{3b} are combined together to form ═O, substituted or unsubstituted C₃₋₇ cycloalkyl, or substituted or unsubstituted 3 to 7 membered heterocycloalkyl;
R^{a} and R^{b} are each independently selected from the group consisting of hydrogen and C₁₋₃ alkyl; and
subscript p is an integer from 1-6.

9. The method of claim 1, wherein encapsulating the therapeutic agent comprises:
(a) admixing the therapeutic agent with a solution of the 1,2-diol modified multi-arm PEG polymer backbone of formula (II) to form a therapeutic agent diol-PEG admixture; and
(b) adding a solution of the PBA modified multi-arm PEG polymer backbone of formula (I) to the therapeutic agent diol-PEG admixture to form the dynamic polymeric hydrogel composition with the therapeutic agent encapsulated therein, thereby stabilizing the encapsulated therapeutic agent.

10.-11. (canceled)

12. The method of claim 1, wherein the therapeutic agent is selected from the group consisting of an enzyme, cell therapy, antibiotic, anesthetic, antibody, growth factor, human embryonic cells, protein, hormone, anti-inflammatory agent, analgesic, cardiac agent, vaccine, and psychotropic agent.

13. (canceled)

14. A method for releasing a stabilized therapeutic agent encapsulated in the dynamic polymeric hydrogel composition of claim 1 comprising:
(i) adding a sugar solution to the dynamic polymeric hydrogel composition with the encapsulated therapeutic agent; or
(ii) lowering the pH of the dynamic polymeric hydrogel composition with the encapsulated therapeutic agent, thereby releasing the stabilized therapeutic agent from the dynamic polymeric hydrogel composition.

15. The method of claim 14, wherein the stabilized therapeutic agent is administered to a patient in need thereof after releasing the stabilized therapeutic agent from the dynamic polymeric hydrogel composition.

16. A dynamic polymeric hydrogel composition comprising:
(a) a therapeutic agent selected from the group consisting of an enzyme, cell therapy, antibiotic, anesthetic, antibody, growth factor, human embryonic cells, protein, hormone, anti-inflammatory agent, analgesic, cardiac agent, vaccine, and psychotropic agent;
wherein the therapeutic agent is present in an amount of from about 0.10 mg/mL to about 100 mg/mL; and
(b) a combination of a phenylboronic acid (PBA) modified multi-arm polyethylene glycol (PEG) polymer backbone of formula (I) and a 1,2-diol modified multi-arm polyethylene glycol (PEG) polymer backbone of formula (II): 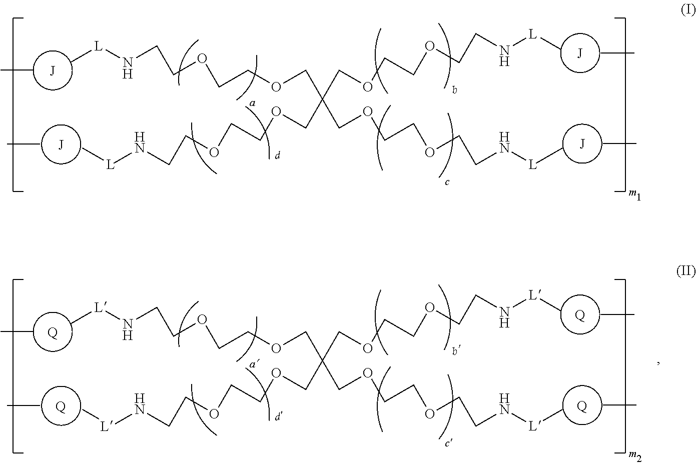
wherein
subscripts a, a′, b, b′, c, c′, d, and d′ are each independently an integer selected from 1 to 500;
subscripts m₁ and m₂ are each independently an integer selected from 10 to 20,000;
linkers L and L′ are each independently selected from a bond, —C(O)—, substituted or unsubstituted alkylene, substituted or unsubstituted —C(O)-alkylene, and substituted or unsubstituted heteroalkylene;
each J is a phenylboronic acid derivative having a pKa of less than 7.8; and
each Q is a 1,2-diol moiety; and
wherein the PBA modified multi-arm PEG polymer backbone and the 1,2-diol modified multi-arm PEG polymer backbone are reversibly covalently cross-linked through the phenylboronic acid derivatives and the 1,2-diol groups.

17. The composition of claim 16, wherein
subscripts a, a′, b, b′, c, c′, d, and d′ are each independently an integer selected from 10 to 250;
subscripts m₁ and m₂ are each independently an integer selected from 25 to 10,000;
each linker L is selected from a bond, —C(O)—, substituted or unsubstituted C₁₋₆ alkylene, and unsubstituted —C(O)—C₁₋₆ alkylene, wherein substituted C₁₋₆ alkylene is substituted with at least one substituent selected from —OH, —NH₂, —SH, —CN, —CF₃, —COOH, —C(O)NH₂, halogen, unsubstituted C₁₋₃ alkyl, and substituted C₁₋₃ alkyl; and
each linker L′ is selected from a bond, —C(O)—, unsubstituted C₁₋₆ alkylene, and unsubstituted —C(O)—C₁₋₆ alkylene.

18. The composition of claim 16, wherein the phenylboronic acid derivative of the PBA modified multi-arm PEG polymer backbone comprises a phenylboronic acid group of formula (III): 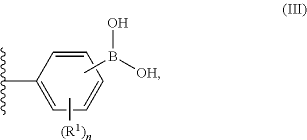
wherein
each R¹ is each independently selected from the group consisting of substituted or unsubstituted alkyl, substituted or unsubstituted heteroalkyl, halogen, —CN, —OH, —NO₂, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a};
R^{a} and R^{b} are each independently selected from the group consisting of hydrogen and C₁₋₆ alkyl; and
subscript n is an integer from 0-4;
wherein the pKa of the phenylboronic acid group is less than 7.8.

19. The composition of claim 18, wherein
each R¹ is each independently selected from the group consisting of substituted or unsubstituted C₁₋₆ alkyl, C₁₋₆ alkoxy, C₁₋₆ haloalkoxy, halogen, —CN, —OH, —NO₂, substituted or unsubstituted phenyl, —NR^{a}R^{b}, —CH₂NR^{a}R^{b}, —C(O)R^{a}, —C(O)OR^{a}, and —C(O)NR^{a}R^{b};
R^{a} and R^{b} are each independently selected from the group consisting of hydrogen and C₁₋₆ alkyl; and
subscript n is an integer from 0-3;
wherein the pKa of the phenylboronic acid group is between about 3.5 and about 7.4.

20. The composition of claim 16, wherein the phenylboronic acid derivative of the PBA modified multi-arm PEG polymer backbone comprises a phenylboronic acid group of formula (IIIA) or formula (IIIB): 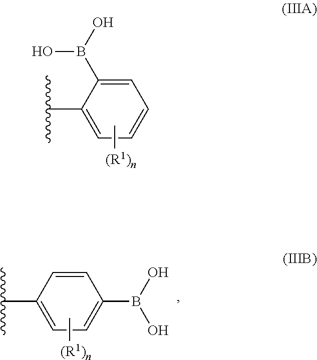
wherein
each R¹ is each independently selected from the group consisting of substituted or unsubstituted C₁₋₃ alkyl, trifluoromethyl, methoxy, ethoxy, fluoro, chloro, bromo, iodo, —CN, —OH, —NO₂, phenyl, benzyl, —NR^{a}R^{b}, —CH₂NR^{a}R^{b}, —C(O)R^{a}, and —C(O)OR^{a};
R^{a} and R^{b} are each independently selected from the group consisting of hydrogen and C₁₋₃ alkyl; and
subscript n is an integer from 0-2;
wherein the pKa of the phenylboronic acid group is between about 4.0 and about 7.2.

21. The composition of claim 16, wherein the 1,2-diol moiety of the 1,2-diol modified multi-arm PEG polymer backbone comprises a 1,2-cis-diol group of formula (IV): 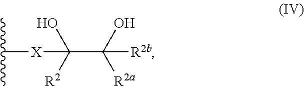
wherein
R², R^{2a}, and R^{2b} are each independently selected from the group consisting of hydrogen, substituted or unsubstituted alkyl, substituted or unsubstituted heteroalkyl, halogen, —CN, —OH, substituted or unsubstituted cycloalkyl, substituted or unsubstituted heterocycloalkyl, substituted or unsubstituted aryl, substituted or unsubstituted heteroaryl, —NR^{a}R^{b}, —CH₂NR^{a}R^{b}, —C(O)R^{a}, —C(O)OR^{a}, —C(O)NR^{a}R^{b}, —SR^{a}, —S(O)R^{a}, and —S(O)₂R^{a};
wherein, optionally, R² and one of R²a or R^{2b} are combined together to form a substituted or unsubstituted cycloalkyl, substituted or unsubstituted heterocycloalkyl, substituted or unsubstituted aryl, or substituted or unsubstituted heteroaryl;
R^{a} and R^{b} are each independently selected from the group consisting of hydrogen and C₁₋₆ alkyl; and
X is selected from the group consisting of a bond, substituted or unsubstituted alkylene, substituted or unsubstituted heteroalkylene, and —C(O)—.

22. The composition of claim 21, wherein
R², R^{2a}, and R^{2b} are each independently selected from the group consisting of hydrogen, substituted or unsubstituted C₁₋₆ alkyl, C₁₋₆ alkoxy, C₁₋₆ haloalkoxy, halogen, —CN, —OH, substituted or unsubstituted phenyl, —NR^{a}R^{b}, —CH₂NR^{a}R^{b}, —C(O)R^{a}, —C(O)OR^{a}, and —C(O)NR^{a}R^{b};
wherein, optionally, R² and one of R²a or R^{2b} are combined together to form a substituted or unsubstituted C₃₋₇ cycloalkyl, or substituted or unsubstituted 3 to 7 membered heterocycloalkyl;
R^{a} and R^{b} are each independently selected from the group consisting of hydrogen and C₁₋₆ alkyl; and
X is selected from the group consisting of a bond, C₁₋₆ alkylene, and C₁₋₆ alkoxy; wherein, optionally, the C₁₋₆ alkylene is substituted with 1-4 substituents each independently selected from the group consisting of —OH, C₁₋₃ alkyl, —C(O)R^{a}, and —C(O)—.

23. The composition of claim 16, wherein the 1,2-diol moiety of the 1,2-diol modified multi-arm PEG polymer backbone comprises a 1,2-cis-diol group of formula (IVA): 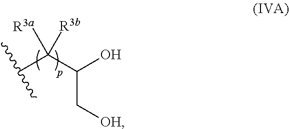
wherein
R^{3a} and R^{3b} are each independently selected from the group consisting of hydrogen, substituted or unsubstituted C₁₋₃ alkyl, methoxy, ethoxy, fluoro, chloro, bromo, iodo, —OH, —NR^{a}R^{b}, —CH₂NR^{a}R^{b}, —C(O)R^{a}, and —C(O)OR^{a};
wherein, optionally, one or more of R^{3a} and R^{3b} are combined together to form ═O, substituted or unsubstituted C₃₋₇ cycloalkyl, or substituted or unsubstituted 3 to 7 membered heterocycloalkyl;
R^{a} and R^{b} are each independently selected from the group consisting of hydrogen and C₁₋₃ alkyl; and
subscript p is an integer from 1-6.
